(19) United States
(12) Patent Application Publication
(10) Pub. No.: US 2025/0177556 A1
(43) Pub. Date: Jun. 5, 2025

(54) LIPID NANOPARTICLES FOR DELIVERY TO THE EYE OR EAR

(71) Applicants: Trustees of Tufts College, Medford, MA (US); Massachusetts Eye and Ear Infirmary, Boston, MA (US)

(72) Inventors: Qiaobing Xu, Lexington, MA (US); Zheng-Yi Chen, Boston, MA (US); Min Qiu, Medford, MA (US); Yamin Li, Somerville, MA (US); Mingqian Huang, Boston, MA (US)

(21) Appl. No.: 18/839,185

(22) PCT Filed: Feb. 16, 2023
(86) PCT No.: PCT/US23/13216

§ 371 Date: Aug. 16, 2024

Related U.S. Application Data
(60) Provisional application No. 63/310,860, filed on Feb. 16, 2022.

(51) Int. Cl. A61K 47/69; A61K 9/00; A61K 38/45; A61K 38/46; A61K 47/64

(52) U.S. Cl. CPC A61K 47/6911; A61K 9/0046; A61K 38/45; A61K 38/465; A61K 47/645

(57) ABSTRACT

Disclosed herein are methods and compositions related to delivery of pharmaceutical agents by lipid nanoparticles (LNPs) to a cell of a target organ (e.g., an eye, an ear) of a subject.

### RELATED APPLICATIONS

This application claims the benefit of priority to U.S. Provisional Patent Application 63/310,860, filed Feb. 16, 2022; the contents of which are hereby incorporated by reference in their entirety.

### GOVERNMENT SUPPORT

This invention was made with government support under grant number UG3 TR002636-01 awarded by the National Institutes of Health. The government has certain rights in the invention.

### BACKGROUND

Genome editing technologies enable the permanent repair of disease-causing genetic mutations. However, the application of this technology has been limited by the technical challenge of achieving safe, effective, and specific in vivo delivery of the CRISPR-Cas9 genome editing components.

More than a hundred gene alleles have been identified as associated with human genetic disorders of the eye and ear. Pharmacological treatments for genetic blindness and deafness are lacking in the clinic due to a lack of an efficient delivery system that can ferry therapeutics into the inner structures of the eye and ear.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

### SUMMARY

Lipid nanoparticles (LNPs) delivered CRISPR-Cas9 genome editing components into neonatal mouse cochlea can restore hearing loss. However, target delivery of therapeutic proteins and nucleic acids to adult mouse eye (e.g., retina) and ear (e.g., cochlea) remain a great challenge. Since fully developed organs in adult mice more closely reflects the physiological conditions of human newborns, there is a great need for developing LNP-based delivery systems for delivering CRISPR-Cas9 genome editing components to treat human congenital blindness and deafness.

In an aspect, the present disclosure provides a composition, comprising a pharmaceutical agent assembled with a lipid composition that comprises an ionizable lipid, wherein the ionizable lipid has an amine head group and at least one hydrophobic tail having a structure of Formula (A):

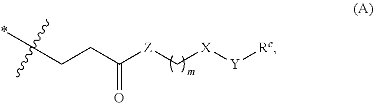

wherein:
   *indicates the point of attachment to N;
   X and Y are independently —CH₂—, —O—, —S—, or —Se—;
   Z is N or O;
   each m is independently 1, 2, 3, 4, or 5; and
   each R^{c} is independently an alkyl, or an alkenyl.

In some embodiments, the lipid composition is capable of delivering the pharmaceutical agent to a plurality of cell types in an ear. In some embodiments, the plurality of cell types comprises at least one cell type. In some embodiments, the plurality of cell types comprises at least two cell types. In some embodiments, when the pharmaceutical agent is delivered to one cell type, the one cell type is not a hair cell. In some embodiments, the plurality of cell types is selected from the group consisting of inner hair cells (IHC), outer hair cells (OHC), Hensen cells (HeCs), Deiter cells (DC), outer sulcus cells (OSCs), inner pillar cells (IPC), and outer pillar cells (OPC).

In some embodiments, the lipid composition is capable of delivering the pharmaceutical agent to a plurality of regions in an eye. In some embodiments, the plurality of regions is selected from the group consisting of a retina, a retinal pigment epithelium (RPE), photoreceptors, bipolar cells, ganglion cells, horizontal cells, and amacrine cells of the subject.

In some embodiments, the ionizable lipid comprises a structure of Formula (I):

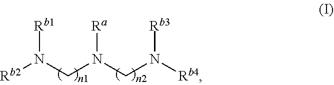

or a pharmaceutically acceptable salt thereof, wherein:
   i) R^{a} is an alkyl;
   ii) n1 and n2 are each independently 1, 2, 3, or 4; and
   iii) R^{b1}, R^{b2}, R^{b3} and R^{b4} are each independently H, or

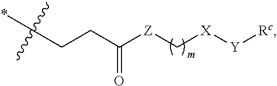

wherein at least one of R^{b1}, R^{b2}, R^{b3} and R^{b4} is not H.

In some embodiments, the amine head group is selected from the group consisting of

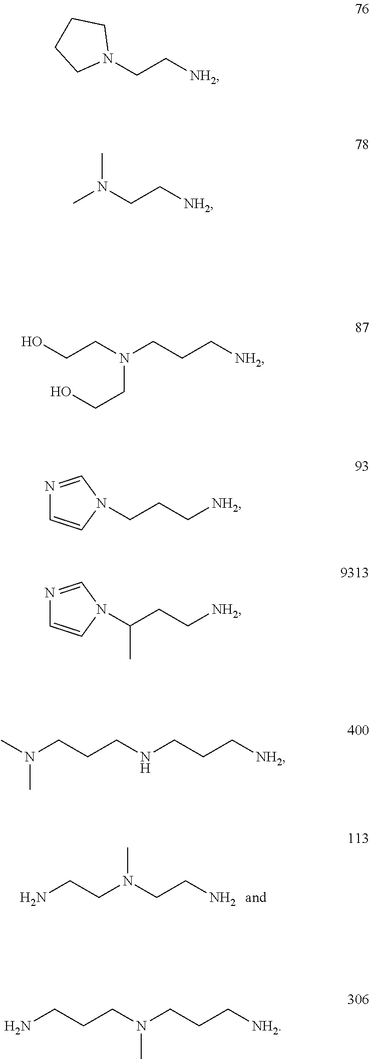

In some embodiments, R^{c} is C₄-C₂₀ alkyl. In some embodiments, R^{c} is C₄-C₂₀ alkenyl.

In some embodiments, the lipid composition further comprises a steroid. In some embodiments, the steroid is cholesterol or a cholesterol derivative. In some embodiments, the lipid composition further comprises a helper lipid. In some embodiments, the helper lipid is 1,2-dioleoyl-sn-glycero-3-phosphoethanolamine (DOPE) or 1,2-dioleoyl-sn-glycero-3-phosphocholine (DOPC). In some embodiments, the lipid composition further comprises a polymer conjugated lipid. In some embodiments, the polymer conjugated lipid is a PEG conjugated lipid. In some embodiments, the polymer conjugated lipid is 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k). In some embodiments, the lipid composition comprises a transactivator of transcription (TAT) peptide modification.

In some embodiments, the lipid composition further comprises a steroid, a helper lipid, and a polymer conjugated polymer. In some embodiments, the ionizable lipid is present in the lipid composition at a weight percentage from about 30% to about 90%. In some embodiments, steroid is present in the lipid composition at a weight percentage from about 10% to about 40%. In some embodiments, the helper lipid is present in the lipid composition at a weight percentage from about 1% to about 20%. In some embodiments, the polymer conjugated lipid is present in the lipid composition at a weight percentage from about 1% to about 20%. In some embodiments, the weight ratio of the ionizable lipid/steroid/helper lipid/polymer conjugated lipid is about 16/4/1/1. In some embodiments, the weight ratio of the ionizable lipid/steroid/helper lipid/polymer conjugated lipid is about 16.7/4/2.1/1.

In some embodiments, the lipid composition further comprises a steroid and a helper lipid. In some embodiments, the ionizable lipid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the ionizable lipid/steroid/helper lipid is about 2/1/1.

In some embodiments, the lipid composition further comprises an excipient. In some embodiments, the excipient is selected from the group consisting of (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, 80-EC16, and Bile salts. In some embodiments, the excipient is present in the composition at a weight percentage from about 5% to about 60%.

In some embodiments, the pharmaceutical agent is a therapeutic agent, a gene modulating agent, or a vaccine. In some embodiments, the pharmaceutical agent comprises a polynucleotide, an oligonucleotide, a polypeptide, an oligopeptide, a small molecule compound, or any combination thereof. In some embodiments, the pharmaceutical agent comprises: (a) a gene modulating moiety configured to specifically bind at least a portion of a target gene or a gene product thereof, or (b) a polynucleotide that encodes the gene modulating moiety of (a). In some embodiments, the gene modulating moiety comprises a guide nucleic acid configured to complex with at least a portion of the target gene or the gene product thereof, or a polynucleotide sequence that encodes the guide nucleic acid. In some embodiments, the gene modulating moiety comprises a heterologous endonuclease (e.g., a clustered regularly interspaced short palindromic repeats (CRISPR)-associated (Cas) nuclease) or a polynucleotide (e.g., a messenger ribonucleic acid (mRNA)) comprising a sequence that encodes the heterologous endonuclease.

In some embodiments, the ionizable lipid comprises at least two hydrophobic tails, wherein not all hydrophobic tails are identical. In some embodiments, the ionizable lipid comprises at least two hydrophobic tails, wherein two or more hydrophobic tails are identical.

In another aspect, the present disclosure provides a method for delivering a pharmaceutical agent to an ear or an ear cell in a subject in need thereof, the method comprising administering any composition disclosed herein. In some embodiments, the administering is through canalostomy or cochleostomy. In some embodiments, the administering is through systemic administration. In some embodiments, the administering comprises administering to inner ear.

In another aspect, the present disclosure provides a method for delivering a pharmaceutical agent to an eye or an eye cell in a subject in need thereof, the method comprising administering any one of the compositions disclosed herein. In some embodiments, the administering comprises administering to a retina. In some embodiments, the administering comprises administering to retinal pigment epithelial cells. In some embodiments, administering comprises injecting into subretinal space. In some embodiments, the administering is through systemic administration.

In another aspect, the present disclosure provides a method for delivering a pharmaceutical agent to a target organ (e.g., ear or eye) in a subject in need thereof, the method comprising administering to the subject the pharmaceutical agent assembled with a lipid composition that comprises an ionizable lipid, a steroid, a helper lipid, and a polymer conjugated lipid, thereby providing a greater amount or activity of the pharmaceutical agent in the target organ therein in the subject as compared to that achieved absent the ionizable lipid.

In another aspect, the present disclosure provides a method for delivering a pharmaceutical agent to a target organ (e.g., ear or eye) in a subject in need thereof, the method comprising administering to the subject the pharmaceutical agent assembled with a lipid composition that comprises an ionizable lipid, a steroid, a helper lipid, and a polymer conjugated lipid, thereby providing a greater amount or activity of the pharmaceutical agent in the target organ therein in the subject as compared to a non-target organ.

In some embodiments, the pharmaceutical agent is administered at a dosage of no more than 3 mg/kg body weight. In some embodiments, the pharmaceutical agent is administered in one or more doses.

In another aspect, the present disclosure provides compositions comprising lipid nanoparticles for delivery of RNA to the eye or ear (e.g., cochlea).

In one aspect, disclosed is a composition (e.g., for preferential delivery to a target organ or a target cell, e.g., for modifying an expression profile of a target gene or a gene product thereof (e.g., a target protein or a functional variant thereof, or a target transcript) in the target organ or the target cell) comprising: a pharmaceutical agent (e.g., a therapeutic agent, a gene modulating agent, or a vaccine) assembled with a lipid composition, wherein the lipid composition comprises a lipidoid having structural Formula (I):

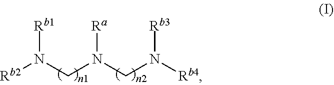

or a pharmaceutically acceptable salt thereof, wherein:
R^{a} is an alkyl (e.g., C₁-C₄ alkyl);
n1 and n2 are each independently 1, 2, 3, or 4; and
R^{b1}, R^{b2}, R^{b3} and R^{b4} are each independently H, or

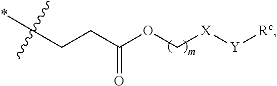

wherein:
   * indicates the point of attachment to N;
   X and Y are independently —CH₂—, —O—, —S—, or —Se—;
   each m is independently 1, 2, 3, 4, or 5; and
   each R^{c} is independently an alkyl (e.g., C₄-C₂₀), or an alkenyl (e.g., C₄-C₂₀,

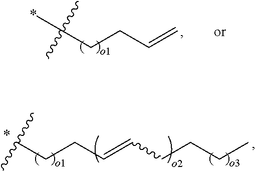

wherein each o1, o2, and o3 is
independently an integer 1-10), * indicates the point of attachment to Y; and wherein at least one of R^{b1}, R^{b2}, R^{b3} and R^{b4} is not H.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure”, “Fig.”, and “FIG.” herein), of which:

FIG. 1 shows development of analogs of 306O12B4 for mRNA delivery.

FIG. 2A shows in vitro Cas9 mRNA delivery.

FIG. 2B shows in vivo fLuc mRNA delivery.

FIGS. 3A-3B show structures and cell types of the inner ear.

FIGS. 4A-4B show LNP-mediated protein and mRNA delivery in adult mouse cochlea for genome engineering. FIG. 4A shows that ionizable lipid was synthesized by conjugating amine head with the hydrophobic tail, and protein or mRNA-loaded LNP was fabricated using ionizable lipid, cholesterol, phospholipid, PEG-lipid, or other excipients. The lower left panel shows a representative TEM image of LNPs. Scale bar=50 nm. FIG. 4B illustrates the administration of LNP formulations into the adult mouse inner ear through canalostomy or cochleostomy. The inner ear includes the semicircular canals and cochlea, and the cochlea is spatially divided into different regions labeled as base, mid-base, mid, mid-apex, and apex. The upper right panel shows the cross-section of the cochlea. SV, scala vestibuli; RM, Reissner's membrane; SM, scala media; OC, organ of Corti; ST, scala tympani. The lower right panel shows the loxP-STOP cassette in the Ai14 mouse genome and Cre- or CRISPR-Cas9-mediated tdT fluorescent reporter protein expression.

FIGS. 5A-5F show R-O16B and R-N16B LNP-mediated (−30)GFP-Cre protein delivery in adult Ai14 mice through canalostomy. FIG. 5A illustrates the chemical structures of R-O16B and R-N16B lipids. FIG. 5B shows the (−30)GFP-Cre protein-loaded LNPs are to be administered into adult Ai14 mouse inner ear through canalostomy. FIG. 5C shows the average hydrodynamic diameters (<Dₕ>) of R-O16B and R-N16B LNPs measured by DLS. FIG. 5D shows the (−30)GFP-Cre protein transfection efficacies of LNPs in HeLa-DsRed cells. FIG. 5E illustrates representative fluorescence images of adult Ai14 mouse cochleae received (−30)GFP-Cre-loaded 113-O16B and 306-O16B LNP formulations. The tdT channel shows delivery of cargo. Scale bar=100 μm. FIG. 5F shows a schematic illustration of the (−30)GFP-Cre/R-O16B LNP-induced tdT-positive cell populations in the cochlea (labeled in grey), which are mainly located in the BM and Lim.

FIG. 6A shows the chemical structures of cholesterol, DOPE, and PEG2k-DSPE. FIG. 6B shows the delivery of (−30)GFP-Cre protein with 87-O16B and 400-O16B LNPs into Ai14 adult mouse cochlea via canalostomy. The tdT signal shows delivery of cargo. Scale bar=100 μm.

FIG. 7 shows the delivery of (−30)GFP-Cre protein with 306-N16B and 400-N16B LNPs into Ai14 adult mouse cochlea via canalostomy. Scale bar=100 μm.

FIGS. 8A-8C show the characterization of R-O16B and R-N16B LNPs. FIG. 8A shows the PDI values of R-O16B and R-N16B LNPs measured by DLS. FIGS. 8B and 8C shows Zeta-potential and protein encapsulation efficacy of R-O16B and R-N16B LNPs respectively.

FIGS. 9A-9H show R-O17X LNP-mediated Cre mRNA delivery in adult Ai14 mice through canalostomy. FIG. 9A shows chemical structures of R-O17X lipids. FIG. 9B shows Cre mRNA-loaded R-O17X LNPs are to be administered into adult Ai14 mouse inner ear through canalostomy. FIG. 9C shows transfection efficacies of Cre mRNA-loaded R-O17X LNPs in HeLa-DsRed cells. FIG. 9D is a schematic illustration of dermal fibroblast isolation from adult Ai14 mouse and fluorescence images of fibroblasts treated with Cre mRNA/Lpf2k, Cre mRNA/76-O17Se, and Cre mRNA/78-O17O LNP formulations. Scale bar=100 μm. FIGS. 9E and 9F are representative fluorescence images of adult Ai14 mouse cochleae received Cre mRNA/76-O17Se and Cre mRNA/78-O17O LNP formulations. Scale bar=100 μm. FIG. 9G. shows excipients (i.e., 80-EC16, HP-β-CD, or SA)-incorporated 76-O17Se LNPs loaded with Cre mRNA are to be administered into adult Ai14 mouse inner ear through canalostomy. FIG. 9H is a schematic illustration of the Cre mRNA/R-O17X LNP-induced tdT-positive cell populations in the cochlea, which are mainly located in the BM and Lim. FIGS. 9I and 9J show the characterization of 76-O17Se and 78-O17O LNPs. In particular, the average hydrodynamic diameter and polydispersity index (PDI) of 76-O17Se and 78-O17O LNPs measured by dynamic light scattering (DLS).

FIG. 10 shows the chemical structures of 80-EC16, HP-β-CD, and SA.

FIG. 11 shows the Delivery of Cre mRNA with 76-O17Se incorporated with 80-EC16, HP-β-CD, or SA into Ai14 adult mouse cochlea via canalostomy. The tdT signal shows delivery of cargo. Scale bar=100 μm.

FIGS. 12A-12E show R-O17X LNP-mediated GFP mRNA delivery in adult CD-1 mice through cochleostomy. FIG. 12A shows GFP mRNA-loaded R-O17X or TAT-R-O17X LNPs are to be administered into adult CD-1 mouse inner ear through cochleostomy. FIG. 12B illustrates representative fluorescence images of adult CD-1 mouse cochlea received GFP mRNA/78-O17O LNP formulations. The lower panel shows cross-section images and the right panel shows cochlea images with low magnification. Scale bar=100 μm. FIG. 12C is a schematic illustration of the Cre mRNA/78-O17O LNP-induced GFP-positive cell populations in the cochlea, which are IPCs and OPCs (labeled in red). FIG. 12D are representative fluorescence images of adult CD-1 mouse cochlea received GFP mRNA/TAT-78-O17O LNP formulations. The lower panel shows cross-section images and the right panel shows cochlea images with low magnification. Green, GFP; Red, Phalloidin; Blue, DAPI. Scale bar=100 μm. FIG. 12E is a schematic illustration of the Cre mRNA/TAT-78-O17O LNP-induced GFP-positive cell populations in the cochlea, which are IHCs and OHCs (labeled in red).

FIG. 13 shows the delivery of GFP mRNA with 78-O17O into CD-1 adult mouse cochlea via cochleostomy. Cyan, MYO7A; Red, SOX2; Green, GFP; Blue, DAPI. Scale bar=100 μm.

FIG. 14 shows the delivery of GFP mRNA with TAT-78-O17O into CD-1 adult mouse cochlea via cochleostomy. Red, Phalloidin; Green, GFP; Blue, DAPI. Scale bar=100 μm.

FIG. 15 shows the delivery of GFP mRNA with TAT-76-O17Se into CD-1 adult mouse cochlea via cochleostomy. Cyan, MYO7A; Red, SOX2; Green, GFP; Blue, DAPI. Scale bar=100 μm. The lower panel shows images in the white square with high magnification.

FIGS. 16A-16E show R-O17X LNP-mediated Cre mRNA delivery in adult Ai14 mice through cochleostomy. FIG. 16A: Cre mRNA-loaded R-O17X or TAT-R-O17X LNPs are to be administered into adult Ai14 mouse inner ear through cochleostomy. Left panels of FIGS. 16B-16E are representative fluorescence images of adult Ai14 mouse cochleae received Cre mRNA-loaded 76-O17Se, TAT-76-O17Se, 78-O17O, and TAT-78-O17O LNP formulations. The lower panels in FIG. 16B and FIG. 16C show cross-section images. The tdT signal shows delivery of cargo. Scale bar=100 μm. Right panels of FIGS. 16B-16E are schematic illustrations of the 76-O17Se, TAT-76-O17Se, 78-O17O and TAT-78O17O LNP-induced tdT-positive cell populations in the Ai14 mouse cochlea.

FIG. 17 shows the delivery of Cre mRNA with 76-O17Se into Ai14 adult mouse cochlea via cochleostomy. The tdT signal shows delivery of cargo. Scale bar=100 μm.

FIG. 18 shows the delivery of Cre mRNA with TAT-76-O17Se into Ai14 adult mouse cochlea via cochleostomy. The tdT signal shows delivery of cargo. Scale bar=100 μm. FIG. 5 shows images in the white square with high magnification.

FIG. 19 shows the delivery of Cre mRNA with 78-O17O into Ai14 adult mouse cochlea via cochleostomy. The tdT signal shows delivery of cargo. Scale bar=100 μm. The lower panel shows images in the white square with high magnification.

FIG. 20 shows the delivery of Cas9 mRNA-sgRNA with 76-O17Se and 78-O17O into Ai14 adult mouse cochlea via cochleostomy. The tdT signal shows delivery of cargo. Scale bar=100 μm.

FIG. 21A-21B shows the characterization of 306-O12B, 113-O12B, and 306-O10S LNPs.

FIG. 21A shows TEM image of 306-O12B LNPs. FIG. 21B shows the average hydrodynamic sizes of LNPs measured by DLS. Scale bar=200 nm.

FIGS. 22A-22M show R-O12B, R-O10X, and R-O12X LNP-mediated CRISPR-Cas9 mRNA-sgRNA delivery in adult Ai14 mice through cochleostomy. FIG. 22A shows the chemical structures of R-O12B, R-O10X, and R-O12X lipids. FIG. 22B are representative TEM images of 113-O12B and 306-O10S LNPs. Scale bar=100 nm. FIG. 22C show Cas9 mRNA-sgRNA-loaded LNPs are to administer into adult Ai14 mouse inner ear through cochleostomy. FIGS. 22D & 22G are representative fluorescence images of adult Ai14 mouse cochleae received (D) 306-O12B and (G) 113-O12B LNP formulations. The tdT signal shows delivery of cargo. Scale bar=100 μm. FIGS. 22E, 22H, and 22J show quantification analysis of tdT-positive cells in the Ai14 cochleae received (E) 306-O12B, (H) 113-O12B, and (J) 306-O10S. FIGS. 22F, 22I, and 22M are schematic illustrations of the (F) 306-O12B, (I) 113-O12B, and (M) 306-OOS LNP-induced tdT-positive cell populations (labeled in red) in the cochlea. FIGS. 22K and 22L show fluorescence image (cross-section image is shown in the lower panel) and quantification analysis of tdT-positive cells in the SV of cochlea received 306-O10S. Scale bar=100 μm.

FIG. 23 shows the structure of certain exemplary lipids.

FIG. 24 shows the delivery of Cas9 mRNA-sgRNA with 306-O12B, 113-O12B, and 306-O10S into Ai14 adult mouse cochlea via cochleostomy tdT signal shows delivery of cargo. Right lower panel shows images of SV. Scale bar=100 μm.

FIG. 25 shows the delivery of Cas9 mRNA-sgRNA with 113-O10S, 113-O12Se, and 113-O10Se, into Ai14 adult mouse cochlea via cochleostomy. The tdT signal shows delivery of cargo.

High magnification images in white squares are also shown. The lower panel of 113-O10Se are cross-section images. Scale bar=100 μm.

FIG. 26 shows layers of cells in eye.

FIGS. 27A-27B show Cre mRNA delivery using novel LNPs (76Se and 78O) in retinas of neonatal and adult mice.

FIG. 28 shows Cas9 mRNA/sgRNA delivery with novel LNP in mouse retina. TdTomato Expression was observed in the RPE of mice delivered by Cas9;sgRNA mixtures formulated with 78O, 113-O12B, 306-O12B, and 306-S10 LNP respectively.

FIG. 29A-29B show 76Se and 78O LNPs delivered Cre mRNA to RPE of neonatal mice.

FIG. 30 shows 78O LNPs delivered Cre mRNA to RPE of adult mice.

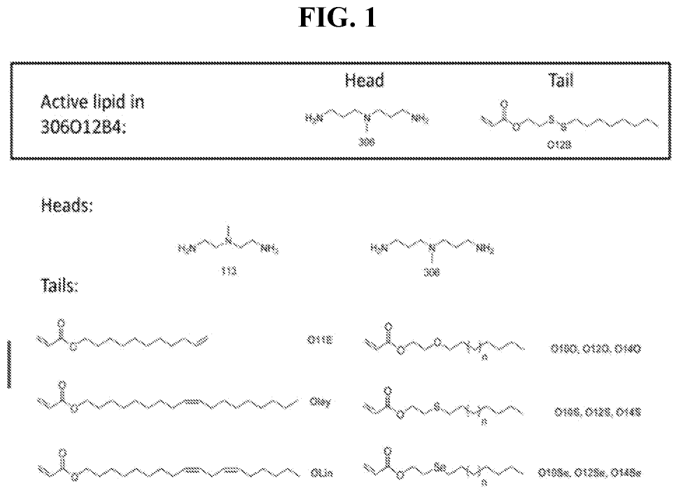

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Provided herein are compositions and methods related to lipids and lipid nanoparticles (LNPs) that can deliver cargos (e.g., pharmaceutical agents, e.g., proteins and/or nucleic acids) into the eyes and/or ears of a subject. In some cases, the proteins comprise a therapeutic protein (e.g., an antibody or fragment thereof), a reporter (e.g., GFP, luciferase), an enzyme (e.g., Cas9, Cre), or a combination thereof. In some cases, the nucleic acids comprise nucleic acids encoding a protein (e.g., mRNA), and nucleic acids capable of hybridizing to nucleic acids (e.g., DNA, RNA) of a subject (e.g., sg RNA, shRNA), or a combination thereof. In some cases, the LNPs provided herein can delivery therapeutic agents into eyes and/or ears of a subject, thereby treating disorders of the eyes and/or ears. In some cases, the disorders of the eyes and/or ears comprises human congenital disorders and genetic disorders of the eyes and/or ears.

In one aspect, the LNPs provided herein can deliver cargos (e.g., a pharmaceutical agent; e.g., proteins and/or nucleic acids) into a plurality of regions of an inner ear of a subject. In some cases, the plurality of regions of an inner ear of a subject comprises scala tympani (ST), scala vestibuli (SV), basilar membrane (BM) of the cochlea, limbus (Lim) of the cochlea, inner hair cells (IHC), outer hair cells (OHC), Deiter cells (DC), inner pillar cells (IPC), outer pillar cells (OPC), Hensen cells (HeCs), outer sulcus cells (OSC), and inner sulcus cells (ISC) of a subject. In some cases, the subject comprises a human. In some cases, the subject comprises an animal. In some cases, the subject comprises a human neonate. In some cases, the subject comprises a neonatal mouse or an adult mouse. The LNPs can deliver the cargos to any one region of the inner ear of a subject. In some cases, the LNPs deliver the cargos to 2, 3, 4, 5, 6, 7, 8, 9, 10 or more regions of the inner ear of a subject. In some cases, the LNPs deliver the cargos to at least 2, at least 3, at least 4, at least 5, at least 6, at least 7, at least 8, at least 9, at least 10 regions of the inner ear of a subject. In some cases, the LNPs deliver the cargos to at most 2, at most 3, at most 4, at most 5, at most 6, at most 7, at most 8, at most 9, at most 10 regions of the inner ear of a subject.

The LNPs provided herein can deliver cargos (e.g., a pharmaceutical agent, proteins, nucleic acids) into a plurality of cell types comprising inner hair cells (IHC), outer hair cells (OHC), Deiter cells (DC), inner pillar cells (IPC), outer pillar cells (OPC), Hensen cells (HeCs), outer sulcus cells (OSC), or inner sulcus cells (ISC). In some cases, the LNPs deliver cargos to 2 or more types of cells of the inner ear, for example, 2, 3, 4, 5, 6, 7, 8 types of cells of the inner ear. In some cases, the LNPs deliver cargos to at least 2, at least 3, at least 4, at least 5, at least 6, at least 7, types of cells of the inner ear. In some cases, the LNPs deliver cargos to at most 2, at most 3, at most 4, at most 5, at most 6, at most 7, types of cells of the inner ear. In some cases, the LNPs delivery cargos to one or more non-sensory cell (non-hair cell) of the inner ear comprising Deiter cells (DC), inner pillar cells (IPC), outer pillar cells (OPC), Hensen cells (HeCs), outer sulcus cells (OSC), or inner sulcus cells (ISC). In some cases, the LNPs deliver cargos to one type of cell of the inner ear. In some cases, the one type of cell of the inner ear is not a hair cell.

In one aspect, the LNPs provided herein deliver cargos (e.g., a pharmaceutical agent; e.g., proteins and/or nucleic acids) into a plurality of regions of an eye of a subject. In some cases, the plurality of regions of an eye of a subject comprises a retina, a retinal pigment epithelium (RPE), photoreceptors, bipolar cells, ganglion cells, horizontal cells, or amacrine cells of a subject. In some cases, the LNPs provided herein deliver cargos into one or more cell types of the eye comprising a retinal pigment epithelium (RPE), photoreceptors, bipolar cells, ganglion cells, horizontal cells, or amacrine cells. In some cases, the LNPs provided herein deliver cargos into 1, 2, 3, 4, 5, 6, or 7 cell types of the eye. In some cases, the LNPs provided herein deliver cargos into at least 1, at least 2, at least 3, at least 4, at least 5, at least 6, or at least 7 cell types of the eye.

### Compositions

In one aspect, disclosed herein is a composition (e.g., for preferential delivery to a target organ or a target cell, e.g., for modifying an expression profile of a target gene or a gene product thereof (e.g., a target protein or a functional variant thereof, or a target transcript) in the target organ or the target cell) comprising: a pharmaceutical agent (e.g., a therapeutic agent, a gene modulating agent, or a vaccine) assembled with a lipid composition.

In some embodiments of the lipid composition of the present application, the lipid composition comprises an ionizable lipid comprising an ionizable cationic group. In some embodiments, the ionizable lipid can comprise one or more groups which is protonated at physiological pH but can be deprotonated and has no charge at a pH above 8, 9, 10, 11, or 12. The ionizable cationic group can comprise one or more protonatable amines which are able to form a cationic group at physiological pH. The cationic ionizable lipid compound can further comprise one or more lipid components such as two or more fatty acids with C₆-C₂₄ alkyl or alkenyl carbon groups. These lipid components or groups can be attached through an ester linkage or may be further added through a Michael addition to a sulfur, an oxygen, a nitrogen, or a selenium atom.

In some embodiments of the lipid composition of the present application, the ionizable cationic lipids refer to lipid and lipid-like molecules with nitrogen atoms that can acquire charges. These molecules with amino or amine groups can have between 1 to 6 hydrophobic chains (or tails), e.g., alkyl or alkenyl groups such as C₆-C₂₄ alkyl or alkenyl groups. In some embodiments, the lipid composition can have 1 hydrophobic chain, 2 hydrophobic chains, 3 hydrophobic chains, 4 hydrophobic chains, 5 hydrophobic chains, 6 or more hydrophobic chains. In some cases, the lipid composition comprises two or more identical hydrophobic chains. In some cases, not all hydrophobic chains of the lipid composition are identical.

In some embodiments of the composition, the lipid composition achieves at least about 1.1-fold greater therapeutic effect in an eye cell or an ear cell provided herein comprising a retinal cell, a sensory ear cell, or a non-sensory ear cell compared to that achieved in other cells. In some embodiments, the ionizable lipid in the composition and achieves at least about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, therapeutic effect in an eye or an ear cell compared to that achieved in other cells.

In some embodiments, the ionizable lipids comprise cationic ionizable lipids. The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids can be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C, ¹⁴C, and ¹⁵N.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments, the ionizable lipid comprises an ammonium group which is positively charged at physiological pH and contains at least two hydrophobic groups. In some embodiments, the ammonium group is positively charged at a pH from about 6 to about 8. In some embodiments, the ionizable cationic lipid comprises at least two C₆-C₂₄ alkyl or alkenyl groups.

In some embodiments, the ionizable lipid comprises a lipidoid having structural Formula (I):

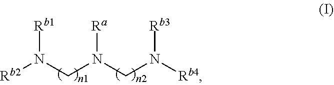

or a pharmaceutically acceptable salt thereof, wherein:
R^{a} is an alkyl (e.g., C₁-C₄ alkyl);
n1 and n2 are each independently 1, 2, 3, or 4; and
R^{b1}, R^{b2}, R^{b3} and R^{b4} are each independently H, or

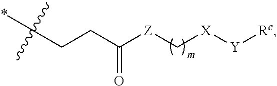

wherein:
   * indicates the point of attachment to N;
   X and Y are independently —CH₂—, —O—, —S—, or —Se—;
   Z is N or O;
   each m is independently 1, 2, 3, 4, or 5; and
   each R^{c} is independently an alkyl (e.g., C₄-C₂₀), or an alkenyl (e.g., C₄-C₂₀,

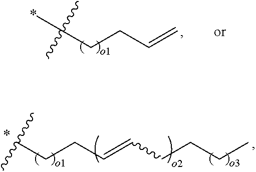

wherein each o1, o2, and o3 is
independently an integer 1-10), * indicates the point of attachment to Y; and wherein at least one of R^{b1}, R^{b2}, R^{b3} and R^{b4} is not H.

In some embodiments, the ionizable lipid comprises a head group and a tail group. In some embodiments the ionizable lipid is formed from a head group selected from:

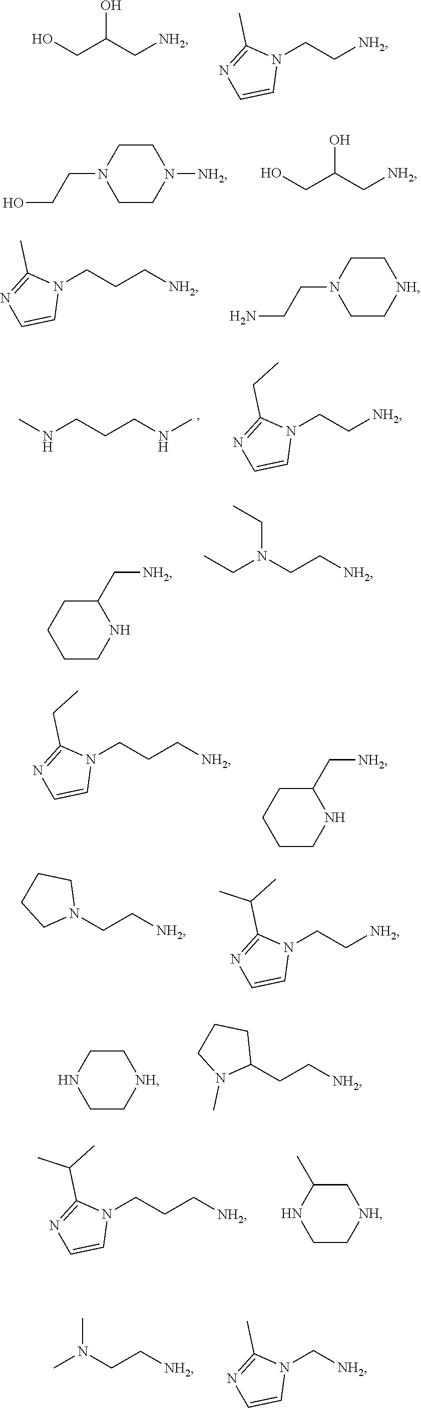

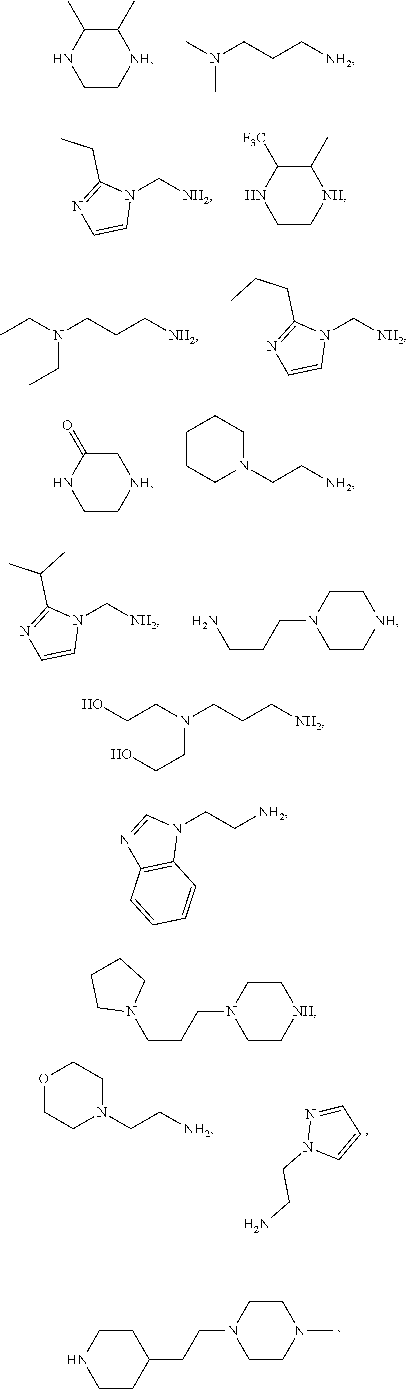

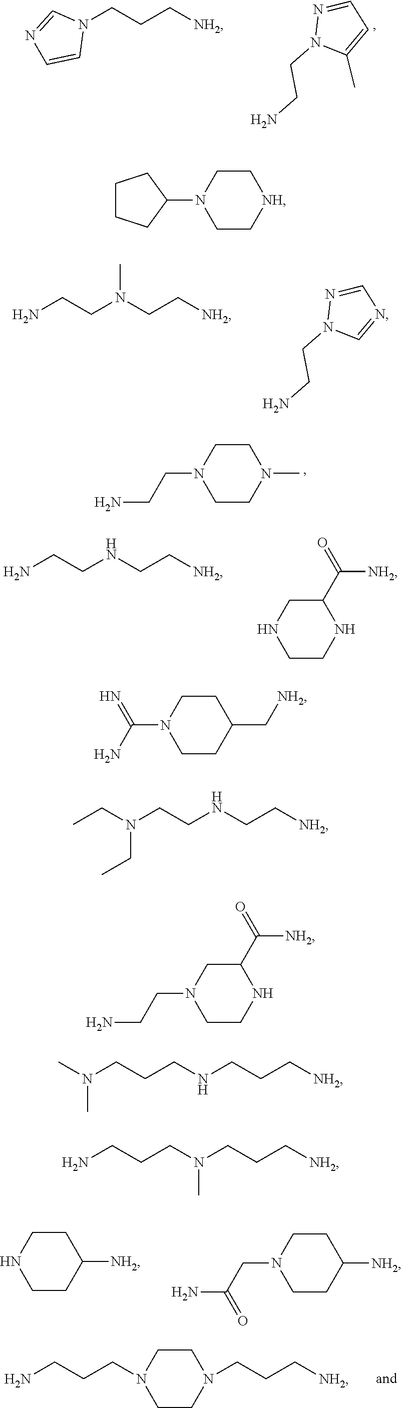

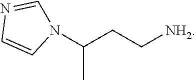

In some embodiments, the hydrophobic tail has a structure of Formula (A):

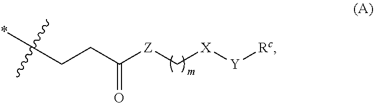

wherein:
*indicates the point of attachment to N;
X and Y are independently —CH₂—, —O—, —S—, or —Se—;
Z is N or O;
each m is independently 1, 2, 3, 4, or 5; and
each R^{c} is independently an alkyl, or an alkenyl.

In some embodiments, the tail group comprises

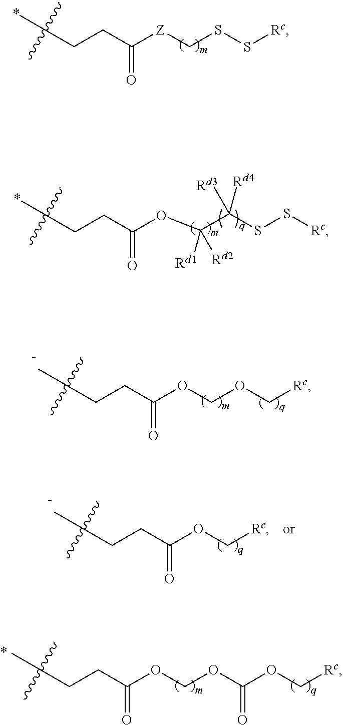

wherein:
R^{d1}, R^{d2}, R^{d3} and R^{d4} are each independently H or C₁-C₄ alkyl, wherein at least one of R^{d1}, R^{d2}, R^{d3} and R^{d4} is not H;
each R^{c} is independently an alkyl or an alkenyl;
each m is independently 1, 2, 3, 4, or 5; and
each q is independently 1, 2, 3, 4, or 5.

In some embodiments, the tail group is formed from 3

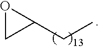

The following are examples of compositions and evaluations of compositions of the disclosure. It is understood that various other embodiments may be practiced, given the general description provided above.

In one aspect, disclosed is a composition (e.g., for preferential delivery to a target organ or a target cell, e.g., for modifying an expression profile of a target gene or a gene product thereof (e.g., a target protein or a functional variant thereof, or a target transcript) in the target organ or the target cell) comprising: a pharmaceutical agent (e.g., a therapeutic agent, a gene modulating agent, or a vaccine) assembled with a lipid composition, wherein the lipid composition comprises a lipidoid having structural Formula (I):

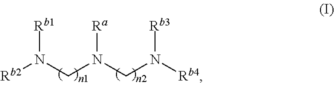

or a pharmaceutically acceptable salt thereof, wherein:
R^{a} is an alkyl (e.g., C₁-C₄ alkyl);
n1 and n2 are each independently 1, 2, 3, or 4; and
R^{b1}, R^{b2}, R^{b3} and R^{b4} are each independently H, or

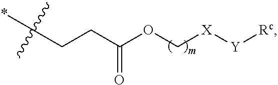

wherein:
   * indicates the point of attachment to N;
   X and Y are independently —CH₂—, —O—, —S—, or —Se—;
   each m is independently 1, 2, 3, 4, or 5; and
   each R^{c} is independently an alkyl (e.g., C₄-C₂₀), or an alkenyl (e.g., C₄-C₂₀,

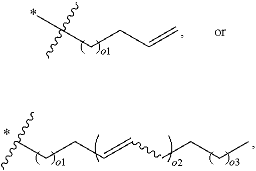

wherein each o1, o2, and o3 is independently an integer 1-10), * indicates the point of attachment to Y; and
   wherein at least one of R^{b1}, R^{b2}, R^{b3} and R^{b4} is not H.

In some embodiments, R^{a} is C₁-C₃ alkyl (e.g., methyl).

In some embodiments, n1 is 1, 2 or 3. In some embodiments, n1 is 2 or 3.

In some embodiments, n2 is 1, 2 or 3. In some embodiments, n2 is 2 or 3 In some embodiments, n1 and n2 are identical.

In some embodiments, at least two (e.g., at least three) of R^{b1}, R^{b2}, R^{b3} and R^{b4} are not H.

In some embodiments, none of R^{b1}, R^{b2}, R^{b3} and R^{b4} is H.

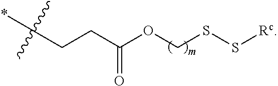

In some embodiments, R^{b1}, R^{b2}, R^{b3} and R^{b4} are

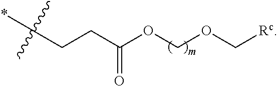

In some embodiments, R^{b1}, R^{b2}, R^{b3} and R^{b4} are

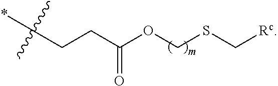

In some embodiments, wherein R^{b1}, R^{b2}, R^{b3} and R^{b4} are

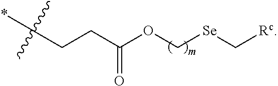

In some embodiments, wherein R^{b1}, R^{b2}, R^{b3} and R^{b4} are

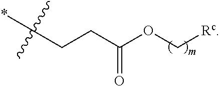

In some embodiments, each R^{c} is independently C₄-C₁₆ (e.g., C₆-C₁₂) alkyl, or C₄-C₂₀ (e.g., C₆-C₁₈) alkenyl. In some embodiments, each R^{c} is independently C₄-C₂₀ alkyl (e.g., C₄-C₁₆ alkyl, such as C₄-C₁₂ alkyl). In some embodiments, each R^{c} is independently C₄-C₂₀ alkenyl (e.g., C₄-C₁₈ alkenyl, such as C₆-C₁₈ alkenyl).

In some embodiments, each R^{c} is independently

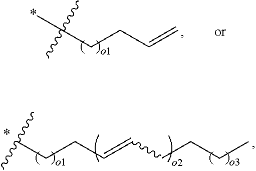

wherein each o1, o2, and o3 is independently an integer 1-10.

In some embodiments, each m is independently 1, 2, 3, or 4. In some embodiments, each m is independently 2, 3, or 4. In some embodiments, each m is 2.

In some embodiments, the lipidoid of Formula (I), or the pharmaceutically acceptable salt thereof, is present in the lipid composition at a molar percentage of no more than about 60% (e.g., no more than about 50%, or no more than about 40%).

In some embodiments, the lipid composition further comprises a steroid or steroid derivative (e.g., cholesterol or a cholesterol derivative).

In some embodiments, the steroid or steroid derivative is present in the lipid composition at a molar percentage of no more than about 50%.

In some embodiments, the lipid composition further comprises a polymer-conjugated lipid (e.g., a poly(ethylene glycol) (PEG) conjugated lipid).

In some embodiments, the polymer-conjugated lipid is present in the lipid composition at a molar percentage of no more than about 10%.

In some embodiments, the lipid composition further comprises a phospholipid (e.g., a phosphoethanolamine lipid, or a phosphocholine lipid).

In some embodiments, the phospholipid is present in the lipid composition at a molar percentage of no more than about 30%.

In some embodiments, the lipidoid of Formula (I), or the pharmaceutically acceptable salt thereof, is present in the composition at a mass or weight ratio to the pharmaceutical agent of about 1:200 to about 200:1 (e.g., about 1:1 to about 100:1).

In some embodiments, the pharmaceutical agent comprises a polynucleotide (e.g., a messenger ribonucleic acid (mRNA)), an oligonucleotide, a polypeptide (e.g., a protein), an oligopeptide, a small molecule compound, or any combination thereof (e.g., configured to (e.g., up- or down-) regulate a target gene or a gene product thereof (e.g., a target protein or a functional variant thereof, or a target transcript) (e.g., in a target organ or a target cell)).

In some embodiments, the pharmaceutical agent comprises a polynucleotide (e.g., a messenger ribonucleic acid (mRNA)) that encodes or is configured to (e.g., up- or down) regulate a target gene or a gene product thereof (e.g., a target protein or a functional variant thereof, or a target transcript, e.g., in a target organ or a target cell).

In some embodiments, the pharmaceutical agent comprises:
(a) a gene modulating moiety configured to specifically bind at least a portion of a target gene or a gene product thereof (e.g., a target protein or a functional variant thereof, or a target transcript); or
(b) a polynucleotide (e.g., a messenger ribonucleic acid (mRNA)) that encodes the gene modulating moiety of (a).

In some embodiments, the gene modulating moiety comprises a guide nucleic acid configured to complex with at least a portion of the target gene or the gene product thereof, or a polynucleotide sequence that encodes the guide nucleic acid.

In some embodiments, the gene modulating moiety comprises a heterologous endonuclease (e.g., a clustered regularly interspaced short palindromic repeats (CRISPR)-associated (Cas) nuclease) or a polynucleotide (e.g., a messenger ribonucleic acid (mRNA)) comprising a sequence that encodes the heterologous endonuclease.

In some embodiments, the heterologous endonuclease is present in the gene modulating moiety at a mass or weight ratio to the guide nucleic acid of about 1:20 to about 20:1 (e.g., about 1:10 to about 10:1).

In some embodiments, the target gene or the gene product thereof (e.g., the target protein or the functional variant thereof, or the target transcript) is specific to or primarily found in a target organ or a target cell of a subject.

In some embodiments, the target gene or the gene product thereof is associated with a disease or disorder of the target organ or the target cell.

In some embodiments, the gene modulating moiety is configured to provide a modified expression profile of the target gene or the gene product thereof (e.g., the target protein or the functional variant thereof, or the target transcript) in a target organ or a target cell of a subject.

In some embodiments, the target organ is an eye or an ear (e.g., cochlea).

In some embodiments, the target cell is an eye cell or an ear cell.

In some embodiments, wherein the composition is formulated for (e.g., systemic or local) administration.

In certain aspects, the lipidoid of Formula (I), or the pharmaceutically acceptable salt thereof, is a lipidoid having structural Formula (II_{A}), (II_{B}), (II_{C}), (II_{D}), or (II_{E}):

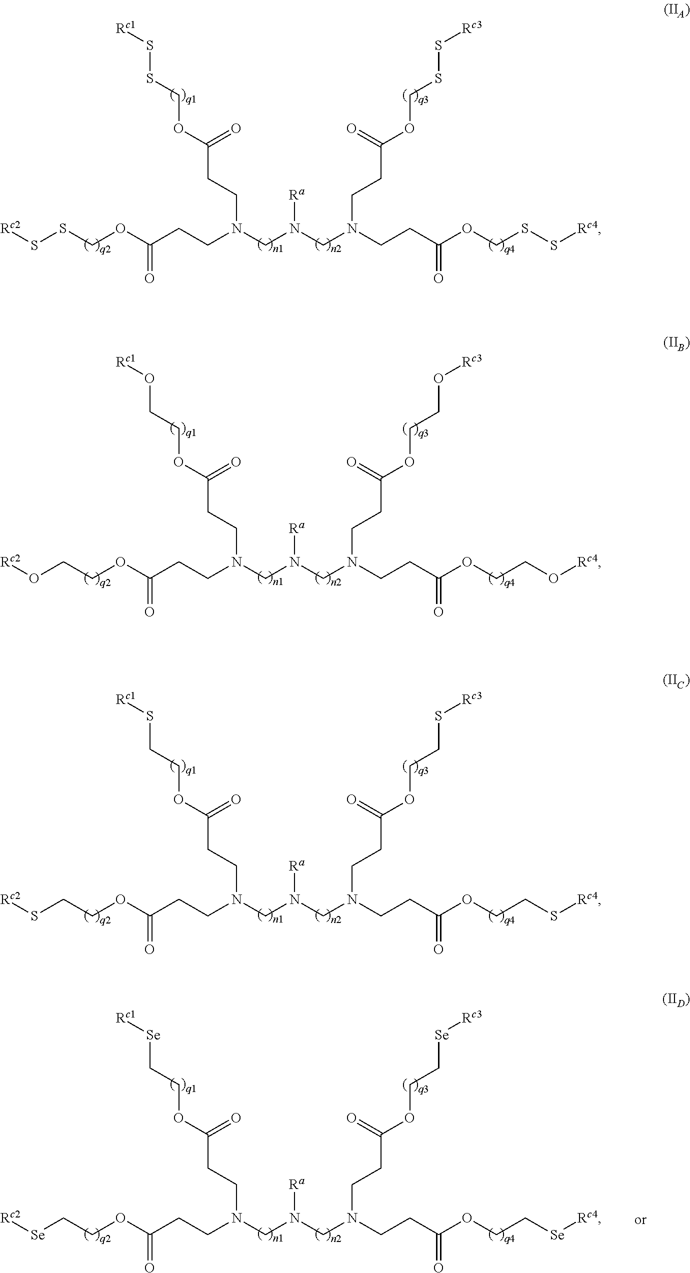

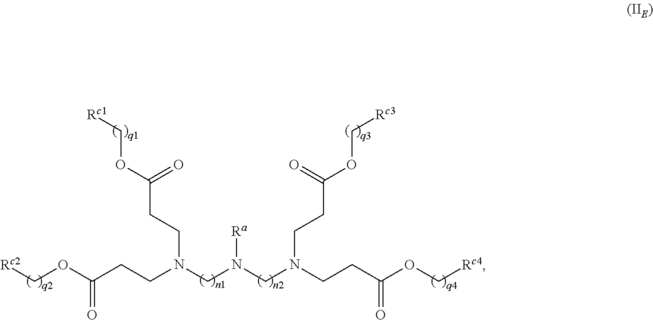

or a pharmaceutically acceptable salt thereof, wherein:
q1, q2, q3 and q4, are each independently 1, 2, 3, or 4; and
R^{c1}, R^{c2}, R^{c3} and R^{c4}, are each independently C₄-C₂₀ alkyl, or C₄-C₂₀ alkenyl.

In some embodiments, the lipidoid of Formula (I), or the pharmaceutically acceptable salt thereof, is the lipidoid of Formula (II_{A}) or the pharmaceutically acceptable salt thereof.

In some embodiments, the lipidoid of Formula (I), or the pharmaceutically acceptable salt thereof, is the lipidoid of Formula (II_{B}) or the pharmaceutically acceptable salt thereof.

In some embodiments, the lipidoid of Formula (I), or the pharmaceutically acceptable salt thereof, is the lipidoid of Formula (II_{C}) or the pharmaceutically acceptable salt thereof.

In some embodiments, the lipidoid of Formula (I), or the pharmaceutically acceptable salt thereof, is the lipidoid of Formula (II_{D}) or the pharmaceutically acceptable salt thereof.

In some embodiments, wherein the lipidoid of Formula (I), or the pharmaceutically acceptable salt thereof, is the lipidoid of Formula (II_{E}) or the pharmaceutically acceptable salt thereof.

In some embodiments, R^{c1}, R^{c2}, R^{c3} and R^{c4} is C₄-C₁₆ (e.g., C₆-C₁₂) alkyl.

In some embodiments, R^{c1}, R^{c2}, R^{c3} and R^{c4} is C₄-C₂₀ (e.g., C₆-C₁₈) alkenyl.

In some embodiments, R^{c1}, R^{c2}, R^{c3} and R^{c4} is

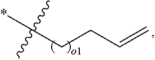

and wherein o1 is 7.

In some embodiments, R^{c1}, R^{c2}, R^{c3} and R^{c4} is

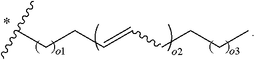

In some embodiments, o1 is 7, o2 is 1, and o3 is 5.

In some embodiments, o1 is 7, o2 is 2, and o3 is 2.

In some embodiments, q1, q2, q3, and q4 is 1, 2, or 3.

In some embodiments, the lipidoid is selected from:

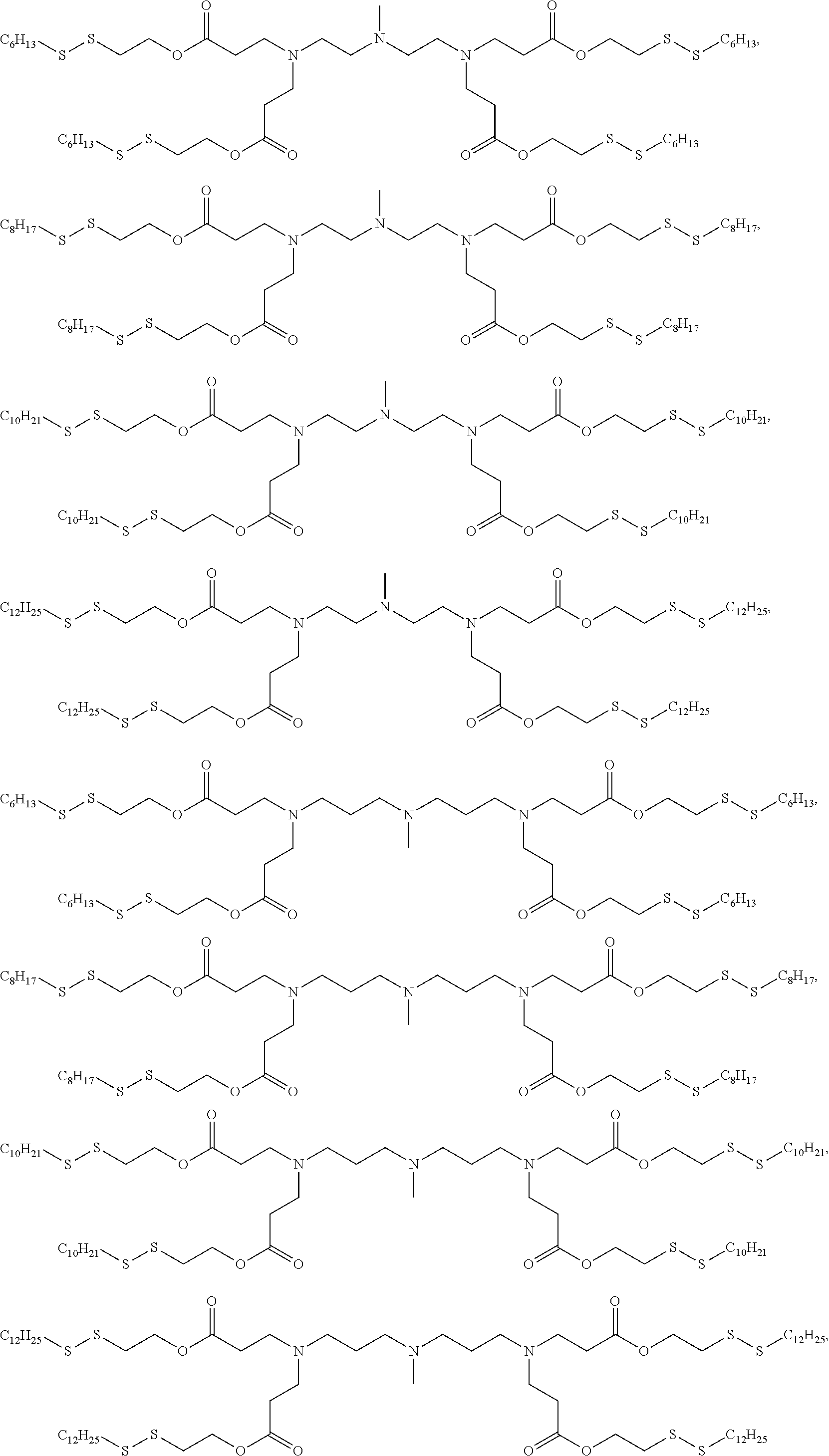

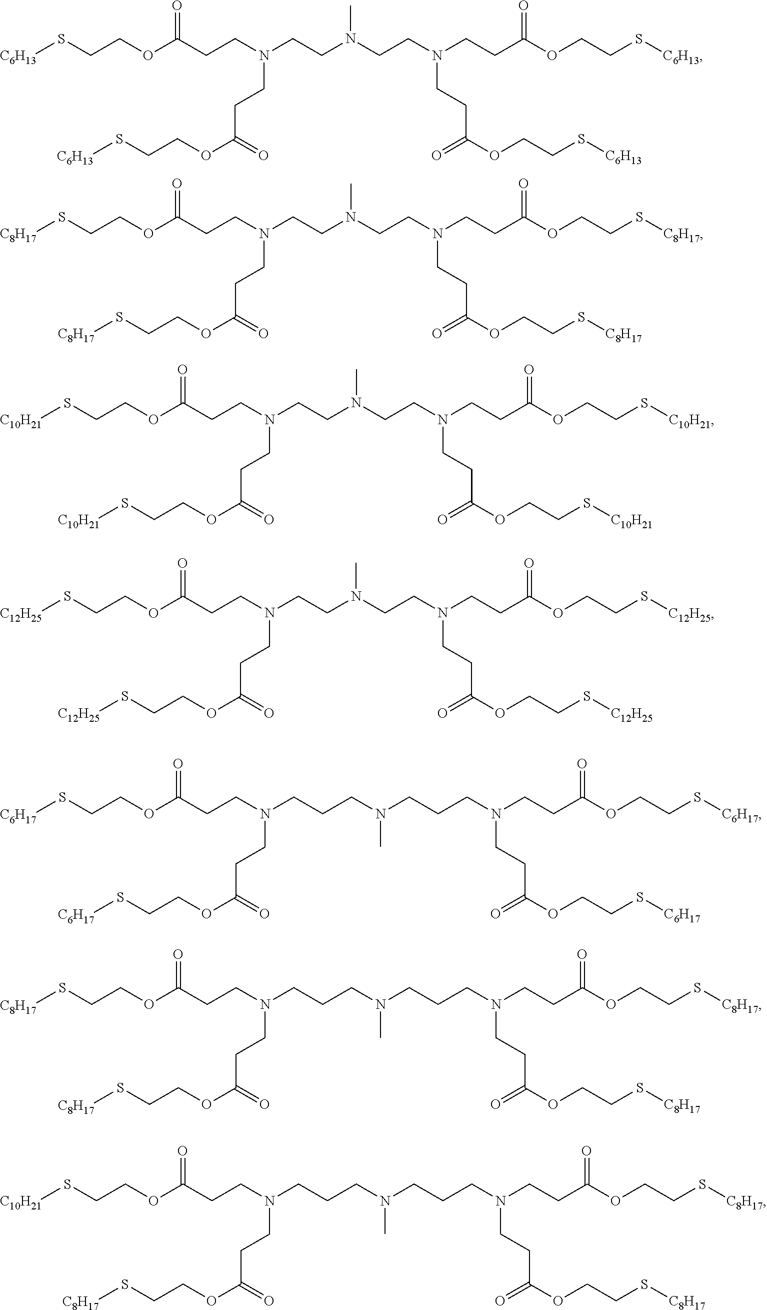

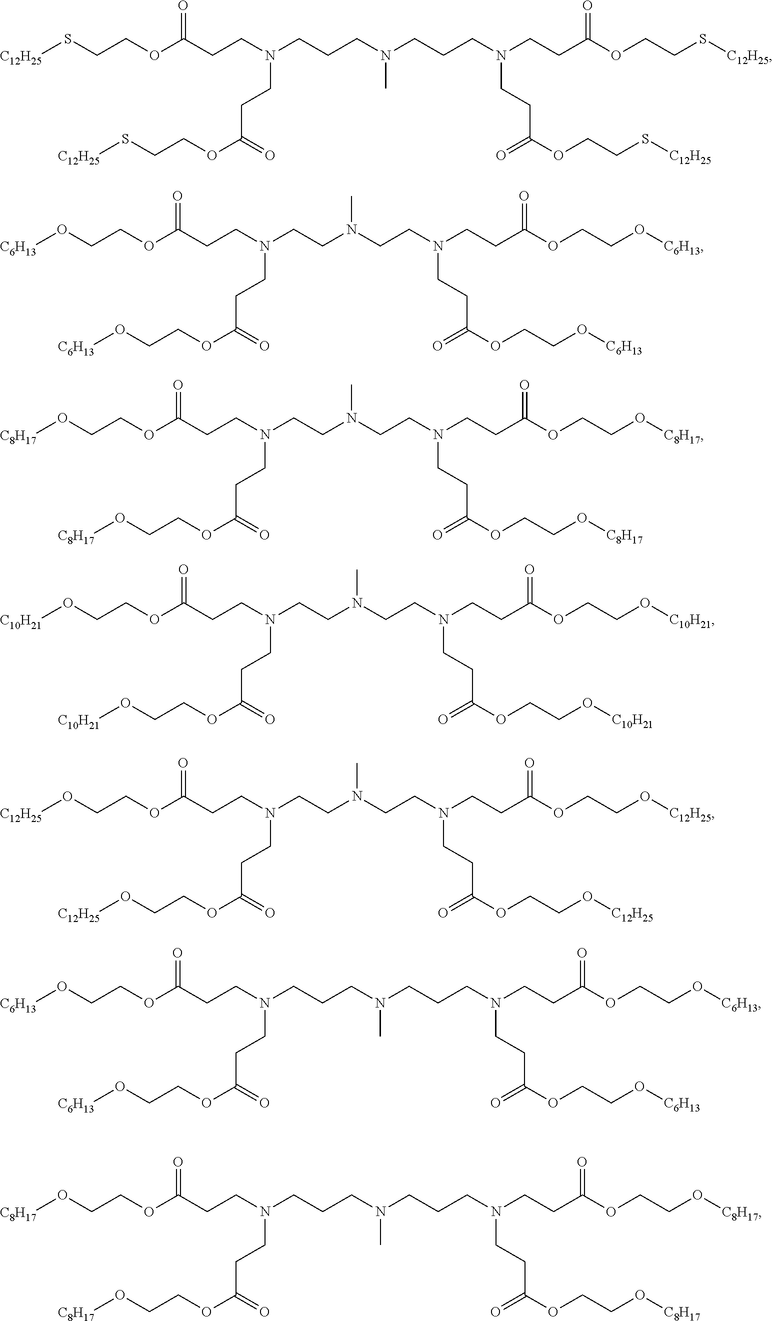

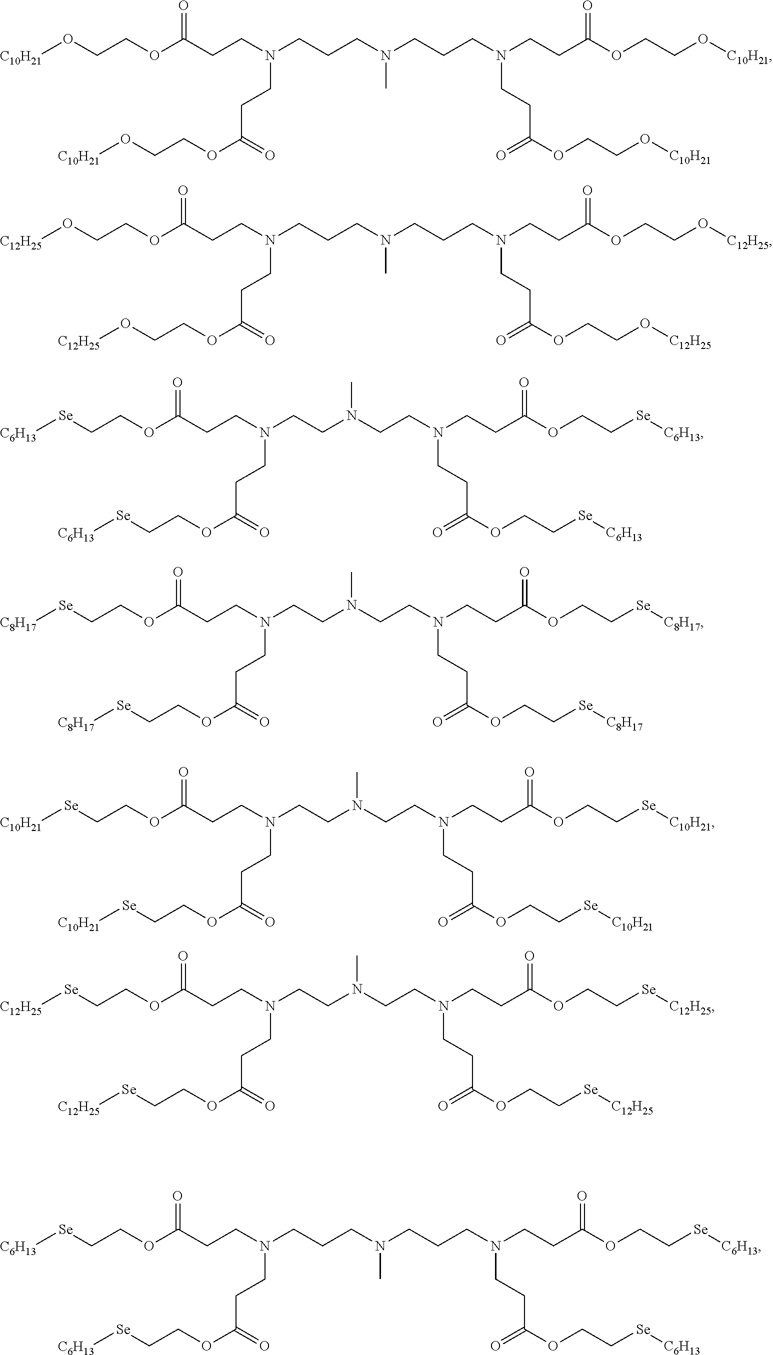

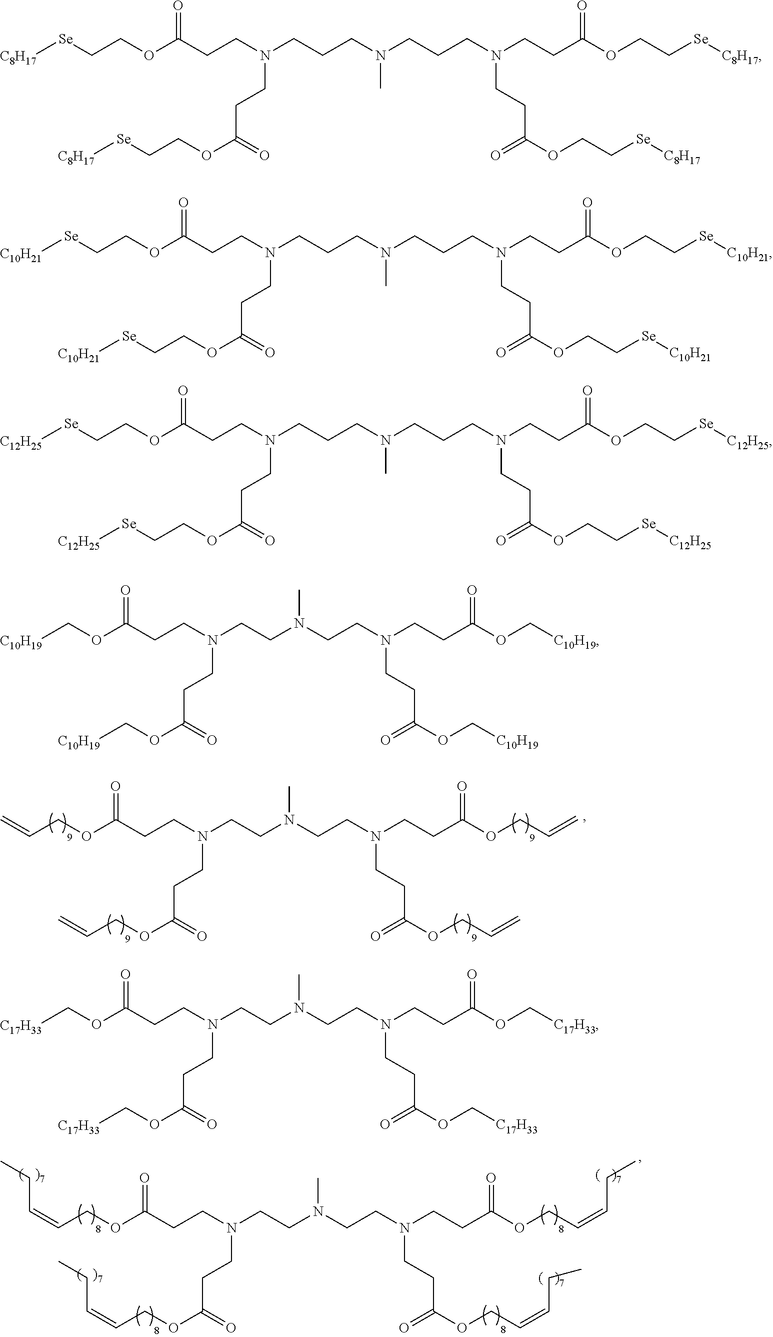

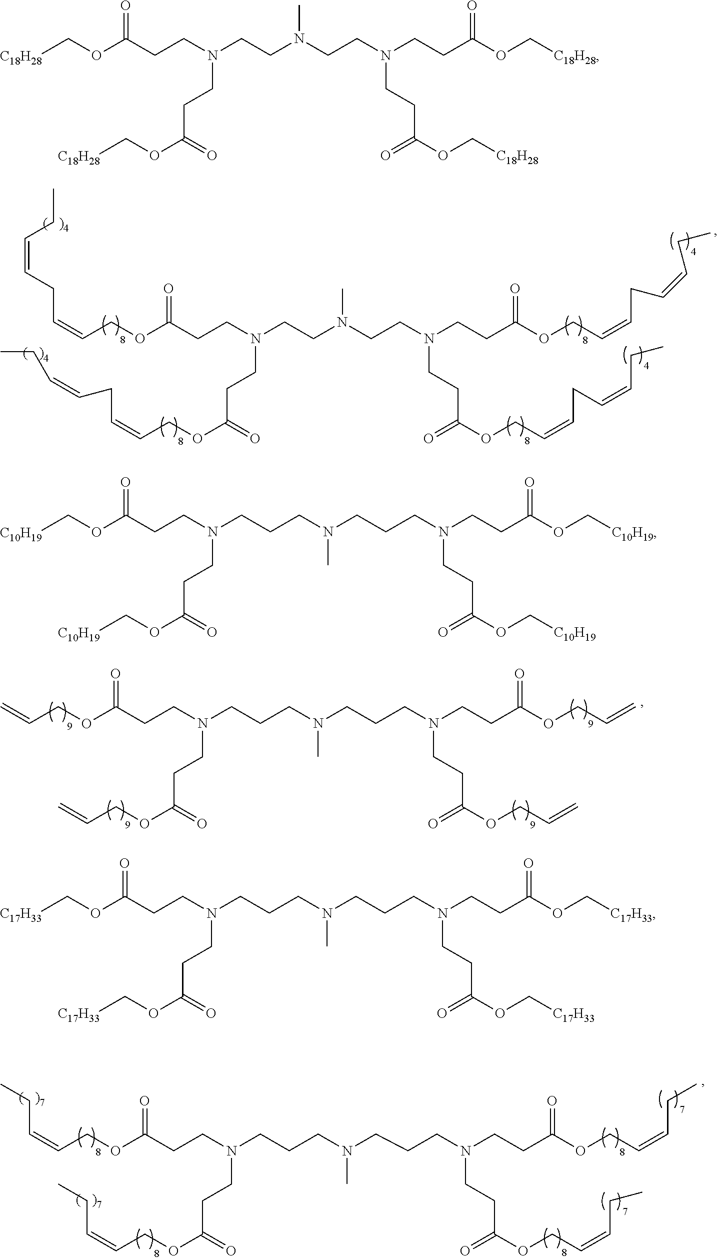

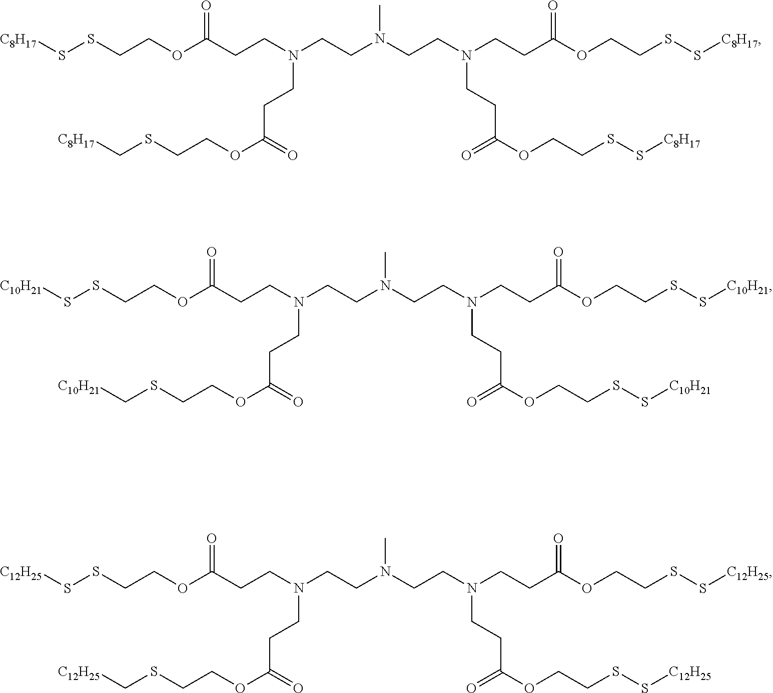

and a pharmaceutically acceptable salt of any of the foregoing.

In some embodiments, the lipidoid of Formula (I) or Formula (II_{A}) is not a lipidoid selected from the group consisting of:

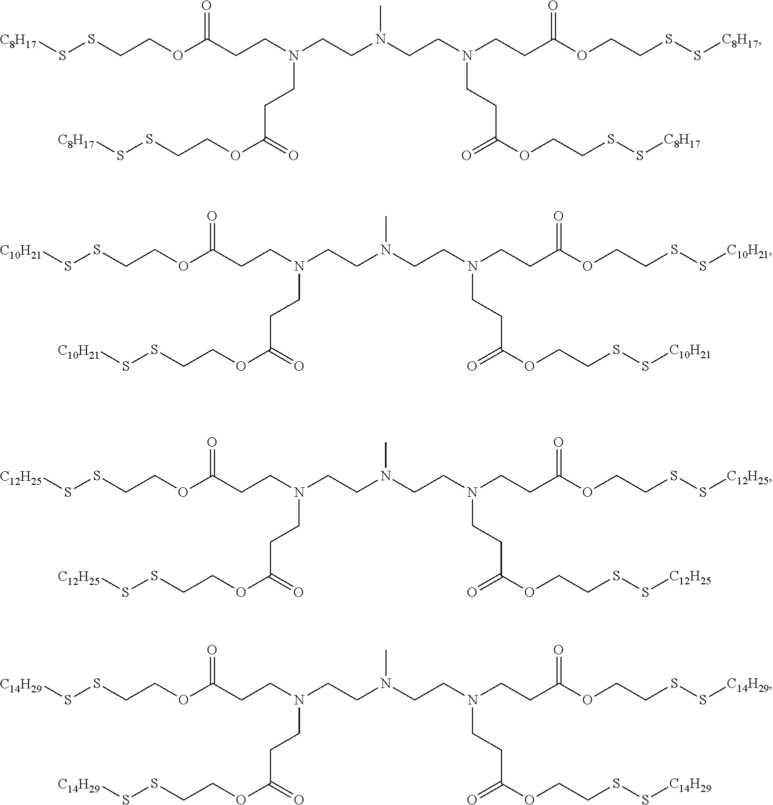

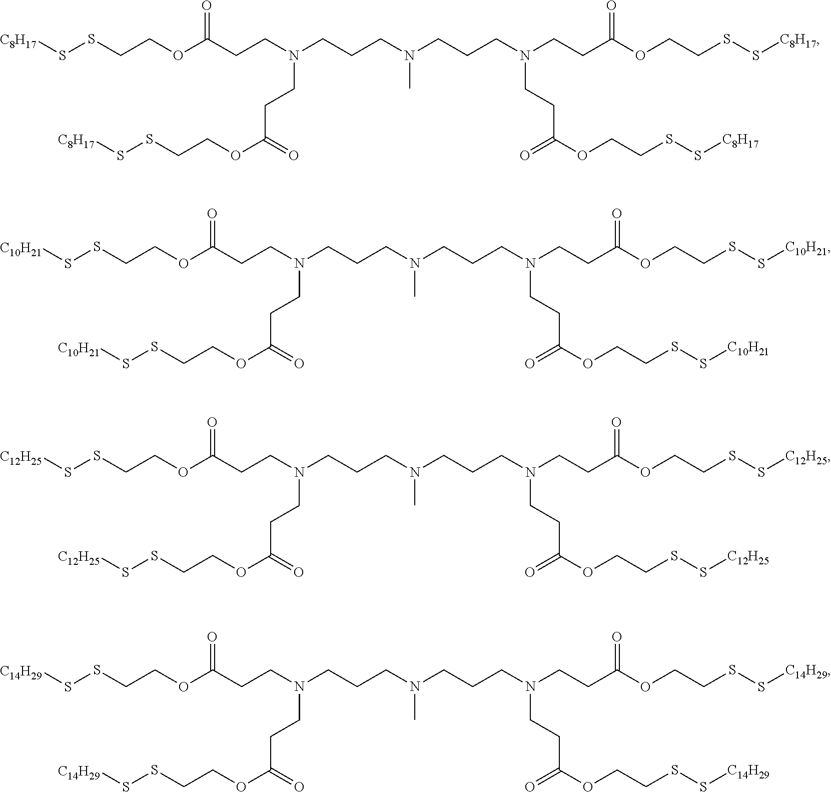

and a pharmaceutically acceptable salt of any of the foregoing.

In certain aspects, the lipidoid of Formula (I), (II_{A}), (II_{B}), (II_{C}), (II_{D}), or (II_{E}), or the pharmaceutically acceptable salt thereof, is present in the lipid composition at a molar percentage of about 20% to about 50%.

In some embodiments, the lipid composition comprises a steroid or steroid derivative at a molar percentage of about 10% to about 50%.

In some embodiments, the lipidoid of Formula (I), (II_{A}), (II_{B}), (II_{C}), (II_{D}), or (II_{E}), or the pharmaceutically acceptable salt thereof, is present in the composition at a mass or weight ratio to the pharmaceutical agent of about 5:1 to about 100:1.

In some embodiments, the composition is for a preferential delivery of the pharmaceutical agent to an eye or an eye cell (e.g., in a subject) as compared to a delivery to a non-eye organ (e.g., an ear) or a non-eye cell (e.g., an ear cell).

In some embodiments, the pharmaceutical agent encodes or is configured to (e.g., up- or down) regulate a target gene or a gene product thereof (e.g., a target protein or a functional variant thereof, or a target transcript) that is specific to or primarily found in the eye or the eye cell.

In some embodiments, the pharmaceutical agent is configured to provide a modified expression profile of the target gene or the gene product thereof (e.g., the target protein or the functional variant thereof, or the target transcript) in the eye or the eye cell.

In some embodiments, the pharmaceutical agent is associated with an eye disease or disorder.

In an aspect, disclosed is a method for preferential delivery of a pharmaceutical agent to an eye or an eye cell in a subject in need thereof, the method comprising administering the composition described herein, thereby providing a (e.g., at least about 2-, 5-, or 10-fold) greater amount, expression or activity of the pharmaceutical agent in the eye or the eye cell of the subject as compared to that achieved in a non-eye organ (e.g., ear) or a non-eye cell (e.g., an ear cell) in the subject.

In some embodiments, the composition provided herein further comprises a steroid. In some embodiments, the steroid comprises a cholesterol or a cholesterol derivative. In some embodiments, the composition provided herein further comprises a helper lipid. In some embodiments, the helper lipid comprises 1,2-dioleoyl-sn-glycero-3-phosphoethanolamine (DOPE) or 1,2-dioleoyl-sn-glycero-3-phosphocholine (DOPC). In some embodiments, the composition provided herein further comprises a polymer conjugated lipid. In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k). In some embodiments, the ionizable lipid is present in the lipid composition at a weight percentage from about 10% to about 20%, about 10% to about 30%, about 10% to about 40%, about 10% to about 50%, about 10% to about 60%, about 10% to about 80%, about 10% to about 90%, from about 20% to about 30%, from about 20% to about 30%, from about 20% to about 40%, from about 20% to about 50%, from about 20% to about 60%, from about 20% to about 70%, from about 20% to about 80%, from about 20% to about 90%, from about 30% to about 40%, from about 30% to about 50%, from about 30% to about 60%, from about 30% to about 70%, from about 30% to about 80%, from about 30% to about 90%, from about 40% to about 50%, from about 40% to about 60%, from about 40% to about 70%, from about 40% to about 80%, from about 40% to about 90%, from about 50% to about 60%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, or from about 80% to about 90%. In some embodiments, the ionizable lipid is present in the lipid composition at a weight percentage of about 10%, about 20%, about 30%, about 40%, about 50%, about 60%, about 70%, about 80%, or about 90%. In some embodiments, the lipid composition further comprises a modification comprising a cell-penetrating peptide. In some cases, the cell-penetrating peptide is transactivator of transcription (TAT).

In some embodiments, the lipid composition comprises an ionizable lipid disclosed in this application, a steroid, a helper lipid, and a polymer conjugated polymer.

In some embodiments, the steroid comprises a cholesterol or a cholesterol derivative. In some embodiments, the helper lipid comprises 1,2-dioleoyl-sn-glycero-3-phosphoethanolamine (DOPE) or 1,2-dioleoyl-sn-glycero-3-phosphocholine (DOPC). In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments, the ionizable lipid is present in the lipid composition at a weight percentage from about 10% to about 20%, about 10% to about 30%, about 10% to about 40%, about 10% to about 50%, about 10% to about 60%, about 10% to about 80%, about 10% to about 90%, from about 20% to about 30%, from about 20% to about 30%, from about 20% to about 40%, from about 20% to about 50%, from about 20% to about 60%, from about 20% to about 70%, from about 20% to about 80%, from about 20% to about 90%, from about 30% to about 40%, from about 30% to about 50%, from about 30% to about 60%, from about 30% to about 70%, from about 30% to about 80%, from about 30% to about 90%, from about 40% to about 50%, from about 40% to about 60%, from about 40% to about 70%, from about 40% to about 80%, from about 40% to about 90%, from about 50% to about 60%, from about 50% to about 70%, from about 50% to about 80%, from about 50% to about 90%, from about 60% to about 70%, from about 60% to about 80%, from about 60% to about 90%, from about 70% to about 80%, from about 70% to about 90%, or from about 80% to about 90%.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments, the weight ratio of the ionizable lipid/steroid/helper lipid/polymer conjugated lipid is about 14/4/1/1, about 15/4/1/1, about 16/4/1/1, about 17/4/1/1, about 18/4/1/1, about 19/4/1/1, about 20/4/1/1, about 14/4/2/1, about 15/4/2/1, about 16/4/2/1, about 16.7/4/2/1, about 17/4/2/1, about 18/4/2/1, about 19/4/2/1, or about 20/4/2/1.

In some embodiments, the lipid composition comprises an ionizable lipid disclosed in this application, a steroid and a helper lipid. In some embodiments, the ionizable lipid is present in the lipid composition at a weight percentage from about 30% to about 90%. In some embodiments, the helper lipid is present in the lipid composition at a weight percentage from about 5% to about 40%. In some embodiments, the steroid is present in the lipid composition at a weight percentage from about 5% to about 40%. In some embodiments, the weight ratio of the ionizable lipid/steroid/helper lipid is about 1/1/1, 2/1/1, about 3/1/1, about 4/1/1, about 5/1/1, about 6/1/1, about 2/2/1, about 3/2/1, about 4/2/1, about 5/2/1, or about 6/2/1.

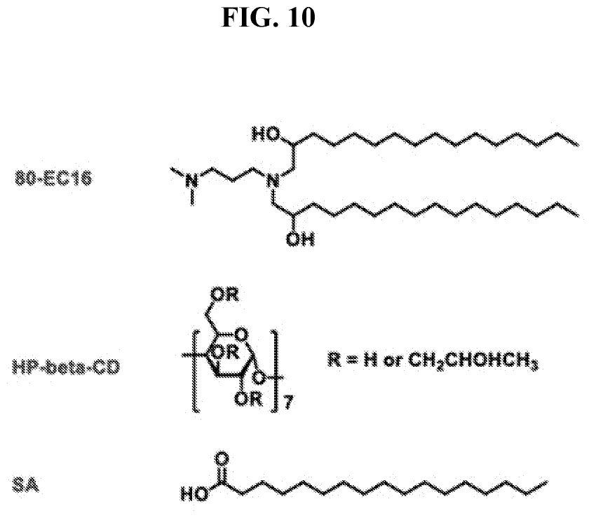

In some embodiments, the lipid composition further comprises an excipient. The excipient can comprise 80-EC16 (see FIG. 10), (2-hydroxypropyl)-β-cyclodextrin ((HP-β-CD), stearic acid, Perfluoroundecanoic, Saponin, Mannitol, Borneol, Amikacin-EC16, Kanamycin-EC16, Neomycin-EC16, or Bile salts. In some embodiments, the excipient is present in the composition at a weight percentage from about 5% to about 60%. In some embodiments, the excipient is present in the composition at a weight percentage from about 1% to about 70%, from about 5% to about 60%, from about 5% to about 50%, from about 5% to about 40%, from about 5% to about 30%, from about 10% to about 50%, from about 10% to about 40%, from about 10% to about 30%, from about 10% to about 20%.

### Additional Lipids

In some embodiments of the lipid composition of the present application, the lipid composition further comprises an additional lipid comprising a steroid or a steroid derivative, a PEG lipid, and a helper lipid (e.g., phospholipids or other zwitterionic lipids).

In some embodiments of the lipid composition of the present application, the lipid composition further comprises a helper lipid. In some embodiments, the helper lipid comprises a lipid that contributes to the stability or delivery efficiency of the lipid compositions. In some embodiments, the helper lipid comprises a zwitterionic lipid. In some embodiments, the helper lipid comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge. In some embodiments of the lipid compositions, the phospholipid is not an ethylphosphocholine. In some embodiments, the helper lipid can comprise 1,2-dioleoyl-sn-glycero-3-phosphoethanolamine (DOPE) or 1,2-dioleoyl-sn-glycero-3-phosphocholine (DOPC).

In some embodiments of the lipid composition of the present application, the compositions may further comprise a molar percentage of the phospholipid to the total lipid composition from about 5 to about 30.

In some embodiments, the helper lipid is present in the lipid composition at a weight percentage from about 1% to about 5%, from about 1% to about 10%, from about 1% to about 20%, from about 5% to about 10%, from about 5% to about 20%, or from about 10% to about 20%.

In some embodiments of the lipid composition of the present application, the lipid composition comprises the phospholipid at a molar percentage from about 8% to about 23%. In some embodiments of the lipid composition of the present application, the lipid composition comprises the phospholipid at a molar percentage from about 10% to about 20%. In some embodiments of the lipid composition of the present application, the lipid composition comprises the phospholipid at a molar percentage from about 15% to about 20%. In some embodiments of the lipid composition of the present application, the lipid composition comprises the phospholipid at a molar percentage from about 8% to about 15%. In some embodiments of the lipid composition of the present application, the lipid composition comprises the phospholipid at a molar percentage from about 10% to about 15%. In some embodiments of the lipid composition of the present application, the lipid composition comprises the phospholipid at a molar percentage from about 12% to about 18%. In some embodiments of the lipid composition of the present application, the lipid composition comprises the phospholipid at a molar percentage of at least about 8%, at least about 10%, at least about 12%, at least about 15%, at least about 18%, at least about 20%, or at least about 23%. In some embodiments of the lipid composition of the present application, the lipid composition comprises the phospholipid at a molar percentage of at most about 8%, at most about 10%, at most about 12%, at most about 15%, at most about 18%, at most about 20%, or at most about 23%.

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms.

In one aspect, the ring structure of a steroid comprises three fused cyclohexyl rings and a fused cyclopentyl ring as shown in the formula:

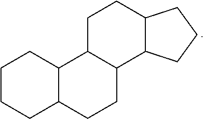 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

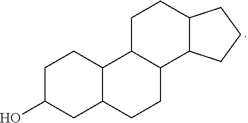 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

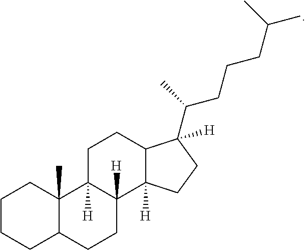

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar percentage of the steroid to the total lipid composition from about 20 to about 60. In some embodiments, the steroid is present in the lipid composition at a weight percentage from about 10% to about 20%, from about 10% to about 30%, from about 10% to about 40%, from about 20% to about 30%, from about 20% to about 40%, or from about 30% to about 40%.

In some embodiments of the lipid composition of the present application, the lipid composition comprises the steroid or steroid derivative at a molar percentage from about 15% to about 46%. In some embodiments of the lipid composition of the present application, the lipid composition comprises the steroid or steroid derivative at a molar percentage from about 20% to about 40%. In some embodiments of the lipid composition of the present application, the lipid composition comprises the steroid or steroid derivative at a molar percentage from about 25% to about 35%. In some embodiments of the lipid composition of the present application, the lipid composition comprises the steroid or steroid derivative at a molar percentage from about 30% to about 40%. In some embodiments of the lipid composition of the present application, the lipid composition comprises the steroid or steroid derivative at a molar percentage from about 20% to about 30%. In some embodiments of the lipid composition of the present application, the lipid composition comprises the steroid or steroid derivative at a molar percentage of at least about 15%, at least about 20%, at least about 25%, at least about 30%, at least about 35%, at least about 40%, at least about 45%, or at least about 46%. In some embodiments of the lipid composition of the present application, the lipid composition comprises the steroid or steroid derivative at a molar percentage of at most about 15%, at most about 20%, at most about 25%, at most about 30%, at most about 35%, at most about 40%, at most about 45%, or at most about 46%.

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

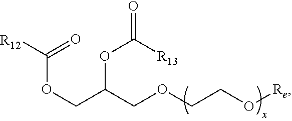

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤8)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

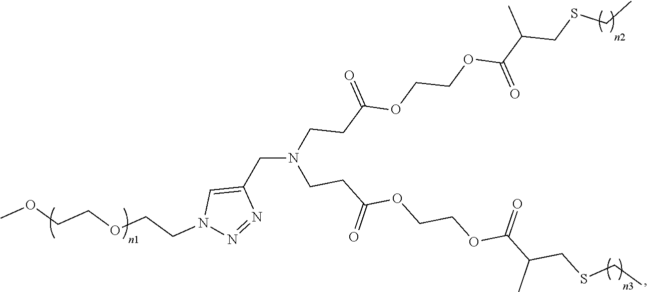

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments, the polymer conjugated lipid comprises 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

In some embodiments of the lipid composition of the present application, the lipid composition comprises the polymer-conjugated lipid at a molar percentage from about 0.5% to about 20%. In some embodiments of the lipid composition of the present application, the lipid composition comprises the polymer-conjugated lipid at a molar percentage from about 1% to about 8%. In some embodiments of the lipid composition of the present application, the lipid composition comprises the polymer-conjugated lipid at a molar percentage from about 2% to about 7%. In some embodiments of the lipid composition of the present application, the lipid composition comprises the polymer-conjugated lipid at a molar percentage from about 3% to about 5%. In some embodiments of the lipid composition of the present application, the lipid composition comprises the polymer-conjugated lipid at a molar percentage from about 5% to about 10%. In some embodiments of the lipid composition of the present application, the lipid composition comprises the polymer-conjugated lipid at a molar percentage of at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5%, or at least about 10%. In some embodiments of the lipid composition of the present application, the lipid composition comprises the polymer-conjugated lipid at a molar percentage of at most about 0.5%, at most about 1%, at most about 1.5%, at most about 2%, at most about 2.5%, at most about 3%, at most about 3.5%, at most about 4%, at most about 4.5%, at most about 5%, at most about 5.5%, at most about 6%, at most about 6.5%, at most about 7%, at most about 7.5%, at most about 8%, at most about 8.5%, at most about 9%, at most about 9.5%, at most about 10%, at most about 15%, or at most 20%.

In some embodiments, the polymer conjugated lipid is present in the lipid composition at a weight percentage from about 1% to about 5%, from about 1% to about 10%, from about 1% to about 20%, from about 5% to about 10%, from about 5% to about 20%, or from about 10% to about 20%.

### Methods

Provided herein are methods for delivering a pharmaceutical agent to an ear or an ear cell in a subject in need thereof, where the method comprises administering the composition provided herein. In some cases, the methods comprise administering the lipids and compositions provided herein through systemic administration. In some cases, the methods comprise administering the lipids and compositions provided herein through local administration. In some cases, local administration comprises administering to an inner ear via canalostomy and/or cochleostomy. Provided herein are methods for delivering a pharmaceutical agent to an eye or an eye cell in a subject in need thereof, where the method comprises administering the composition provided herein. In some cases, the methods comprise administering the lipids and compositions provided herein through systemic administration. In some cases, the methods comprise administering the lipids and compositions provided herein through local administration. In some cases, local administration comprises administering to an eye comprising an injection to the subretinal space.

Another aspect of the methods provided herein relates to delivering of a pharmaceutical agent to a target organ (e.g., ear or eye) in a subject in need thereof, the method comprising administering to the subject the pharmaceutical agent assembled with a lipid composition that comprises an ionizable lipid, a steroid, a helper lipids, and a polymer conjugated lipid, thereby providing a greater amount or activity of the pharmaceutical agent in the target organ therein in the subject as compared to that achieved absent the ionizable lipid.

Another aspect of the methods provided herein relates to delivering a pharmaceutical agent to a target organ (e.g., ear or eye) in a subject in need thereof, the method comprising administering to the subject the pharmaceutical agent assembled with a lipid composition that comprises an ionizable lipid, a steroid, a helper lipid, and a polymer conjugated lipid, thereby providing a greater amount or activity of the pharmaceutical agent in the target organ therein in the subject as compared to a non-target organ.

In some embodiments, the pharmaceutical composition of the present application can be administrated through any suitable routes including, for example, oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the pharmaceutical composition of the present application can be administered in a local rather than systemic manner, for example, via injection of the pharmaceutical composition directly into a targeted tissue (e.g., eye, ear). Local delivery can be affected in various ways, depending on the tissue to be targeted.

In some embodiments, the composition of the present application can be injected into the site of injury, disease manifestation, or pain, for example. In some embodiments, the composition of the present application can even be delivered to the eye or ear by use of creams, drops, or even injection.

In some embodiments, provided herein is a method for potent delivery to a cell of a subject comprising administrating to the subject the pharmaceutical composition as described in the present application. In some embodiments of the method, the pharmaceutical composition comprises a therapeutic agent assembled with a lipid composition as described in the present application, wherein the lipid composition comprises an ionizable lipid.

In some embodiments of any method described herein, the method of delivery of a therapeutic agent to an eye cell comprising administering a composition described herein, thereby providing an effective amount or activity of the therapeutic agent in the eye cell of the subject that is at least 1.1-fold greater than a corresponding amount or activity of the therapeutic agent achieved in a non-eye cell of the subject. In some embodiments, the effective amount or activity of the therapeutic agent in the eye cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the therapeutic agent achieved in a non-eye cell of the subject.

In some embodiments of the method of delivery of the therapeutic agent to an ear cell, the method provides an effective amount or activity of the therapeutic agent in the ear cell of the subject that is at least 1.1-fold greater than a corresponding amount or activity of the therapeutic agent achieved in a non-ear cell of the subject. In some embodiments, the effective amount or activity of the therapeutic agent in the ear cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of the therapeutic agent achieved in a non-ear cell of the subject.

In some embodiments, the methods of delivery comprise administering a lipid composition described herein provides an effective amount or activity of a therapeutic agent at least 1.1-fold greater than a corresponding amount or activity of the therapeutic agent achieved by administering other compositions. In some embodiments, the effective amount or activity of the therapeutic agent results from administering a lipid composition described herein is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the therapeutic agent achieved by administering other compositions.

In some embodiments, the methods of delivery comprise administering a lipid described herein provides an effective amount or activity of a therapeutic agent at least 1.1-fold greater than a corresponding amount or activity of the therapeutic agent achieved by administering other lipids. In some embodiments, the effective amount or activity of the therapeutic agent results from administering a lipid described herein is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of the therapeutic agent achieved by administering other lipids.

In some embodiments, the delivery of the therapeutic to a cell may alter the genome, transcriptome, or expression levels. The cell may be allowed to, or able to, propagate and the alteration may be passed on to the cells generated from the cell that the therapeutic was delivered to. In this manner, the therapeutic effect may be propagated to a larger number of cells. The alteration to the genome, transcriptome or expression level may also persist in a given cell.

In some embodiments, the lipidoid composition delivers the therapeutic agent to the cells of the eye (e.g., retinal cells, RPE, non-retinal cells). In some embodiments, the lipidoid composition delivers the therapeutic agent to the cells of the ear (e.g., sensory hair cells, Deiter cells (DC), inner pillar cells (IPC), outer pillar cells (OPC), Hensen cells (HeCs), outer sulcus cells (OSC), inner sulcus cells (ISC)).

In some embodiments, the methods comprise administering a lipid composition provided herein deliver a cargo (e.g., pharmaceutical agents, proteins, nucleic acids) to a non-sensory cell of the ear comprising Deiter cells (DC), inner pillar cells (IPC), outer pillar cells (OPC), Hensen cells (HeCs), outer sulcus cells (OSC), or inner sulcus cells (ISC). In some embodiments, the methods comprising administering a lipid composition provided herein delivery a cargo to 1, 2, 3, 4, 5, 6, 7, or 8 types of non-sensory cells of the ear. In some embodiments, the methods comprising administering a lipid composition provided herein delivery a cargo to at least 1, at least 2, at least 3, at least 4, at least 5, at least 6, or at least 7, types of non-sensory cells of the ear.

In some embodiments, the methods comprise administering a lipid composition provided herein deliver a cargo (e.g., pharmaceutical agents, proteins, nucleic acids) to two or more cell types of the ear comprising inner hair cells (IHC), outer hair cells (OHC), Deiter cells (DC), inner pillar cells (IPC), outer pillar cells (OPC), Hensen cells (HeCs), outer sulcus cells (OSC), or inner sulcus cells (ISC). In some embodiments, the methods comprising administering a lipid composition provided herein delivery a cargo to 2, 3, 4, 5, 6, 7, or 8 types of cells of the ear. In some embodiments, the methods comprising administering a lipid composition provided herein delivery a cargo to at least 2, at least 3, at least 4, at least 5, at least 6, at least 7, or at least 8 types of cells of the ear.

In some embodiments, the methods comprise administering a lipid composition provided herein deliver a cargo (e.g., pharmaceutical agents, proteins, nucleic acids) to a type of cell of the eye comprising a retinal pigment epithelium (RPE), photoreceptors, bipolar cells, ganglion cells, horizontal cells, or amacrine cells. In some embodiments, the methods comprising administering a lipid composition provided herein delivery a cargo to 1, 2, 3, 4, 5, 6, or 7 types of cells of the eye. In some embodiments, the methods comprising administering a lipid composition provided herein delivery a cargo to at least 1, at least 2, at least 3, at least 4, at least 5, at least 6, or at least 7, types of cells of the eye.

In an aspect, disclosed is a method for preferential delivery of a pharmaceutical agent to an eye or an eye cell in a subject in need thereof, the method comprises administering the composition disclosed herein, thereby providing a (e.g., at least about 2-, 5-, or 10-fold) greater amount, expression or activity of the pharmaceutical agent in the eye or the eye cell of the subject as compared to that achieved with a corresponding reference lipid composition comprising a corresponding reference lipidoid (e.g., having amide-containing tail(s) and/or chalcogens).

In some embodiments, the method modulates a (e.g., at least about 2-, 5-, or 10-fold) greater amount or activity of a target gene or a gene product thereof (e.g., a target protein or a functional variant thereof, or a target transcript) in the eye or the eye cell of the subject as compared to that achieved with a corresponding reference lipid composition comprising a corresponding reference lipidoid (e.g., having amide-containing tail(s)).

In some embodiments, the method provides a modified expression profile of the target gene or the gene product thereof (e.g., the target protein or the functional variant thereof, or the target transcript) in the eye or the eye cell of the subject.

In an aspect, disclosed is a composition, wherein the composition is for a preferential delivery of the pharmaceutical agent to an ear or an ear cell (e.g., in a subject) as compared to a delivery to a non-ear organ (e.g., an eye) or a non-ear cell (e.g., an eye cell).

In some embodiments, the pharmaceutical agent encodes or is configured to (e.g., up- or down) regulate a target gene a gene product thereof (e.g., a target protein or a functional variant thereof, or a target transcript) that is specific to or primarily found in the ear or ear.

In some embodiments, the pharmaceutical agent is configured to provide a modified expression profile of the target gene or the gene product thereof (e.g., the target protein or the functional variant thereof, or the target transcript) in the ear or the ear cell.

In some embodiments, the pharmaceutical agent is associated with a disease or disorder of the eye or ear.

In an aspect, disclosed is a method for preferential delivery of a pharmaceutical agent to an ear or an ear cell in a subject in need thereof, the method comprises administering the composition disclosed herein, thereby providing a (e.g., at least about 2-, 5-, or 10-fold) greater amount, expression or activity of the pharmaceutical agent in the ear or the ear cell of the subject as compared to that achieved in a non-ear organ or a non-ear cell in the subject.

In an aspect, disclosed is a method for preferential delivery of a pharmaceutical agent to an ear or an ear cell in a subject in need thereof, the method comprises administering the composition disclosed herein, thereby providing a (e.g., at least about 2-, 5-, or 10-fold) greater amount, expression or activity of the pharmaceutical agent in the ear or the ear cell of the subject as compared to that achieved with a corresponding reference lipid composition comprising a corresponding reference lipidoid (e.g., having amide-containing tail(s)).

In some embodiments, the method modulates a (e.g., at least about 2-, 5-, or 10-fold) greater amount or activity of a target gene or a gene product thereof (e.g., a target protein or a functional variant thereof, or a target transcript) in the ear or the ear cell of the subject as compared to that achieved with a corresponding reference lipid composition comprising a corresponding reference lipidoid (e.g., having amide-containing tail(s)).

In some embodiments, the method provides a modified expression profile of the target gene or the gene product thereof (e.g., the target protein or the functional variant thereof, or the target transcript) in the ear or the ear cell of the subject.

In some embodiments, the invention relates to the composition or the method for preferential delivery of a pharmaceutical agent to an ear or an ear cell in a subject in need thereof, wherein the lipidoid having structural Formula (I) or Formula (II_{A}) is not a lipidoid selected from the group consisting of:

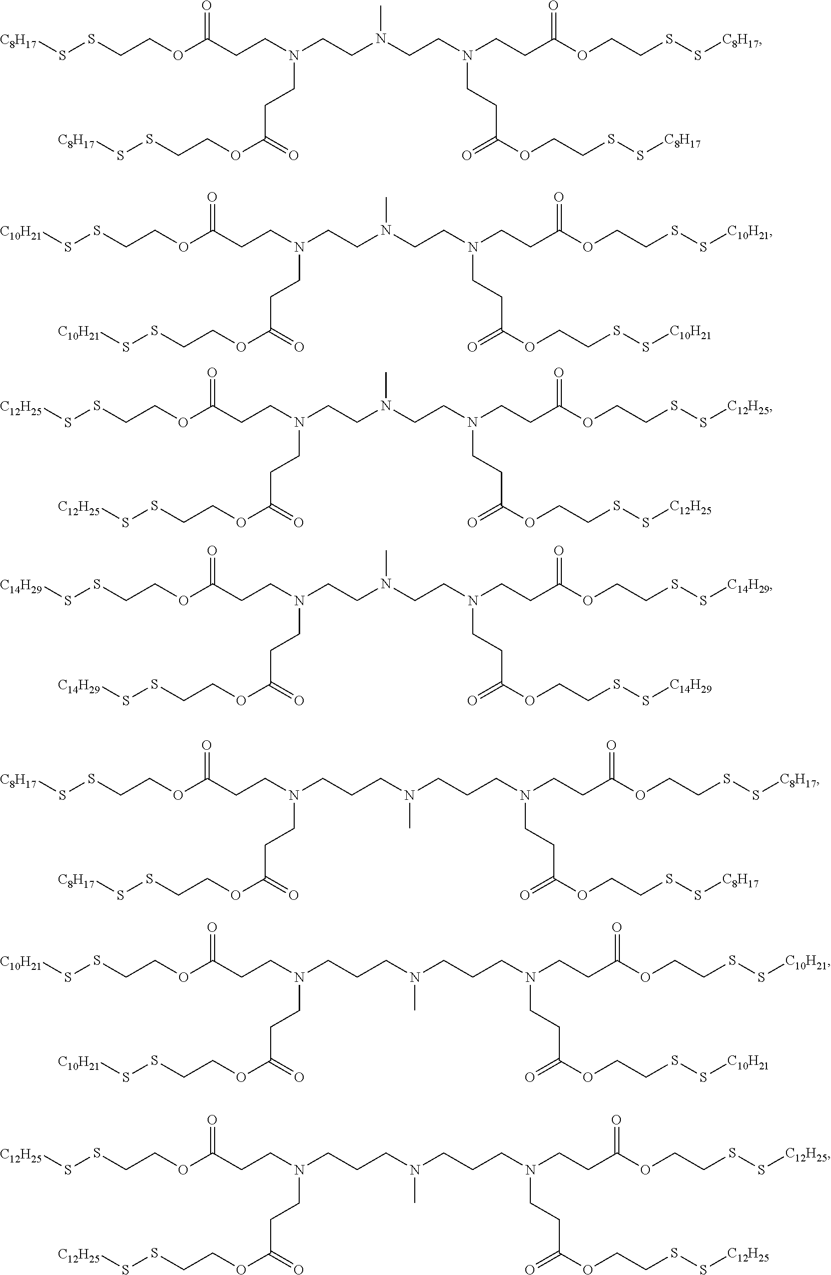

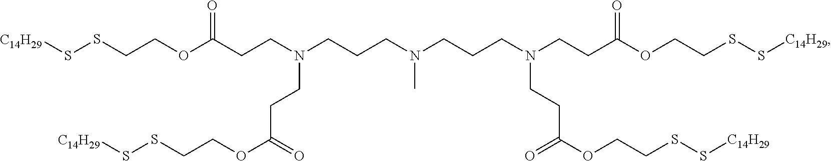

and a pharmaceutically acceptable salt of any of the foregoing.

### Pharmaceutical Compositions

The compositions and methods of the present invention may be utilized to treat an individual in need thereof. The pharmaceutical composition described herein may comprise a therapeutic or prophylactic composition, or any combination thereof. In some embodiments, the lipidoid compositions may be assembled with an antigen, an immune modulator, or any combination thereof. In some embodiments, the individual is a mammal such as a human, or a non-human mammal. When administered to an animal, such as a human, the composition or the lipidoid composition is preferably administered as a pharmaceutical composition comprising, for example, a lipidoid composition of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition such as a lipidoid composition of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In some embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition of the present invention to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective dose of the active lipidoid composition may be administered as one, two, three, four, five, six or more doses administered separately at appropriate intervals throughout the course of treatment, optionally, in unit dosage forms. In some embodiments of the present invention, the active lipidoid composition may be administered two or three times daily. In some embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In some embodiments, lipidoid compositions of the invention may be used alone or conjointly administered with another type of therapeutic agent.

The present disclosure includes the use of pharmaceutically acceptable salts of lipidoid compositions of the invention in the compositions and methods of the present invention. In some embodiments, contemplated salts of the invention include, but are not limited to, alkyl, dialkyl, trialkyl or tetra-alkyl ammonium salts. In some embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In some embodiments, contemplated salts of the invention include, but are not limited to, Na, Ca, K, Mg, Zn or other metal salts. In some embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Therapeutic or Prophylactic Agents

In another aspect, provided herein is a pharmaceutical composition comprising a therapeutic agent (or prophylactic agent) assembled with a lipid composition as described herein.

In some embodiments of the pharmaceutical composition, the therapeutic agent (or prophylactic agent) comprises a compound, a polynucleotide, a polypeptide, or a combination thereof. In some embodiments, the compound, the polynucleotide, the polypeptide, or a combination thereof is exogenous or heterologous to the cell or the subject being treated by the pharmaceutical compositions described herein. In some embodiments, the therapeutic agent (or prophylactic agent) comprises a compound described herein. In some embodiments, the therapeutic agent (or prophylactic agent) comprises a polynucleotide described herein. In some embodiments, the therapeutic agent (or prophylactic agent) comprises a polypeptide described herein. In some embodiments, the therapeutic agent (or prophylactic agent) comprises a compound, a polynucleotide, a polypeptide, or a combination thereof.

In some embodiments, the pharmaceutical composition comprises a therapeutic agent (or prophylactic agent) for treating a disease of the eye or ear. In some embodiments, the therapeutic agent (or prophylactic agent) comprises acetazolamide, acetylcysteine, acyclovir, antazoline and xylometazoline, apraclonidine, atropine, azelastine, azithromycin, betamethasone, betaxolol, bimatoprost, brimonidine, brinzolamide, bromfenac, carbomer, carmellose, carteolol, chloramphenicol, ciprofloxacin, yclopentolate, dexamethasone, diclofenac, dorzolamide, emedastine, epinastine, fluorometholone, flurbiprofen, fusidic, ganciclovir, gentamicin, homatropine, hypromellose, ketorolac, ketotifen, latanoprost, levobunolol, levofloxacin, lodoxamide, loteprednol, moxifloxacin, nedocromil, nepafenac, ofloxacin, olopatadine, pilocarpine, prednisolone, rimexolone, sodium cromoglicate, sodium hyaluronate, tafluprost, timolol, tobramycin, travoprost, or tropicamide.

### Polynucleotides

In some embodiments of the pharmaceutical compositions of the present application, the therapeutic agent (or prophylactic agent) assembled with the lipid composition comprises one or more polynucleotides. The present application is not limited in scope to any particular source, sequence, or type of polynucleotide; however, as one of ordinary skill in the art could readily identify related homologs in various other sources of the polynucleotide including nucleic acids from non-human species (e.g., mouse, rat, rabbit, dog, monkey, gibbon, chimp, ape, baboon, cow, pig, horse, sheep, cat and other species). It is contemplated that the polynucleotide used in the present application can comprises a sequence based upon a naturally occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally occurring sequence. In another embodiment, the polynucleotide comprises nucleic acid sequence that is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

In some embodiments, the polynucleotide used herein may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene; such engineered molecules are sometime referred to as “mini-genes.” At a minimum, these and other nucleic acids of the present application may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the polynucleotide comprises one or more segments comprising a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a or double stranded ribonucleic acid (dsRNA). In some embodiments, the polynucleotide encodes at least one of the therapeutic agents (or prophylactic agent) described herein. In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an endonuclease. Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof.

Some embodiments of the therapeutic agent (or prophylactic agent) provided herein comprise a heterologous polypeptide comprising an actuator moiety. The actuator moiety can be configured to complex with a target polynucleotide corresponding to a target gene. In some embodiments, administration of the therapeutic agent (or prophylactic agent) results in a modified expression or activity of the target gene. The therapeutic agent (or prophylactic agent) may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced short palindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas1O, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or Cul966; any derivative thereof, any variant thereof, or any fragment thereof. In some embodiments, Cas13 can include, but are not limited to, Cas13a, Cas13b, Cas13c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIM5, KYP, SUVR4, Set4, Set1, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. Examples of the genes that may be modified by the guide polynucleotide guided nuclease include CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

In some embodiments, the polynucleotide comprises or encodes at least one mRNA that, upon expression of the mRNA, restores the function of a defective gene in a subject being treated by the pharmaceutical composition described herein.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. In some embodiments, the at least one chemical modification stabilizes scaffold such as a tRNA scaffold. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In some embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof, 4′-C(CH₃)(CH₃)—O-2′ and analogs thereof, 4′-CH₂—N(OCH₃)-2′ and analogs thereof, 4′-CH₂—O—N(CH₃)-2′; 4′-CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group; 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)=N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) β-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In some embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides poylnucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or (S), a or R such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, the polynucleotides described herein comprise or encode at least one tRNA described herein. In some embodiments, the tRNA expressed from the polynucleotide restores the function of at least one defective tRNA in a subject who is being treated by the pharmaceutical composition described herein. In some embodiments, the at least one tRNA expressed by the polynucleotide described herein may include tRNA that encodes alanine, arginine, asparagine, aspartic acid, cysteine, glutamic acid, glutamine, glycine, histidine, hydroxyproline, isoleucine, leucin, lysine, methionine, phenylaniline, proline, pyroglutamic acid, serine, threonine, tryptophan, tyrosine, or valine. In some embodiments, the at least one tRNA expressed by the polynucleotide described herein may include tRNA that encodes arginine, tryptophan, glutamic acid, glutamine, serine, tyrosine, lysine, leucine, glycine, or cysteine.

### Polypeptides

In some embodiments of the pharmaceutical compositions of the present application, the therapeutic agent (or prophylactic agent) assembled with the lipid composition comprises one or more one or more polypeptides. Some polypeptides may include enzymes such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof; any variant thereof; and any fragment thereof. In some embodiments, the nuclease enzyme may include Cas proteins such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, the present application provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin a, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin α, Darbepoetin α, Follicle-stimulating hormone (FSH), and Factor VIII.

In some embodiments, the polypeptide comprises a peptide sequence that is at least partially identical to any of the therapeutic agent (or prophylactic agent) comprising a peptide sequence. For example, the polypeptide may comprise a peptide sequence that is at least partially identical to an antibody (e.g., a monoclonal antibody) for treating a disease such as cancer.

In some embodiments, the polypeptide comprises a peptide or protein that restores the function of a defective protein in a subject being treated by the pharmaceutical composition described herein.

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no more than about 20:1, no more than about 15:1, no more than about 10:1, or no more than about 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no less than about 20:1, no less than about 15:1, no less than about 10:1, or no less than about 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 10:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 10:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 15:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1.

In some embodiments of the pharmaceutical composition of the present application, a molar ratio of the therapeutic agent to total lipids of the lipid composition is from about 1:1 to about 1:100. In some embodiments of the pharmaceutical composition of the present application, a molar ratio of the therapeutic agent to total lipids of the lipid composition is from about 1:1 to about 1:50. In some embodiments of the pharmaceutical composition of the present application, a molar ratio of the therapeutic agent to total lipids of the lipid composition is from about 50:1 to about 1:100. In some embodiments of the pharmaceutical composition of the present application, a molar ratio of the therapeutic agent to total lipids of the lipid composition is from about 1:1 to about 1:20. In some embodiments of the pharmaceutical composition of the present application, a molar ratio of the therapeutic agent to total lipids of the lipid composition is from about 20:1 to about 1:50. In some embodiments of the pharmaceutical composition of the present application, a molar ratio of the therapeutic agent to total lipids of the lipid composition is from about 50:1 to about 1:70. In some embodiments of the pharmaceutical composition of the present application, a molar ratio of the therapeutic agent to total lipids of the lipid composition is from about 70:1 to about 1:100. In some embodiments of the pharmaceutical composition of the present application, a molar ratio of the therapeutic agent to total lipids of the lipid composition is no more than about 1:1, no more than about 1:5, no more than about 1:10, no more than about 1:15, no more than about 1:20, no more than about 1:25, no more than about 1:30, no more than about 1:35, no more than about 1:40, no more than about 1:45, no more than about 1:50, no more than about 1:60, no more than about 1:70, no more than about 1:80, no more than about 1:90, or more than about 1:100. In some embodiments of the pharmaceutical composition of the present application, a molar ratio of the therapeutic agent to total lipids of the lipid composition is no less than about 1:1, no less than about 1:5, no less than about 1:10, no less than about 1:15, no less than about 1:20, no less than about 1:25, no less than about 1:30, no less than about 1:35, no less than about 1:40, no less than about 1:45, no less than about 1:50, no less than about 1:60, no less than about 1:70, no less than about 1:80, no less than about 1:90, or less than about 1:100.

In some embodiments of the pharmaceutical composition of the present application, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% of the therapeutic agent is encapsulated in particles of the lipid compositions.

In some embodiments of the pharmaceutical composition of the present application, the lipid composition comprises a plurality of particles characterized by one or more characteristics of the following: (1) a (e.g., average) size of 100 nanometers (nm) or less; (2) a polydispersity index (PDI) of no more than about 0.2; and (3) a zeta potential of −10 millivolts (mV) to 10 mV.

In some embodiments of the pharmaceutical composition of the present application, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present application, the lipid composition comprises a plurality of particles with a (e.g., average) size from about 50 nanometers (nm) to about 80 nanometers (nm). In some embodiments of the pharmaceutical composition of the present application, the lipid composition comprises a plurality of particles with a (e.g., average) size from about 60 nanometers (nm) to about 80 nanometers (nm). In some embodiments of the pharmaceutical composition of the present application, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present application, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by size exclusion chromatography (SEC). The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments of the pharmaceutical composition of the present application, the lipid composition comprises a plurality of particles with a polydispersity index (PDI) from about 0.05 to about 0.5. In some embodiments of the pharmaceutical composition of the present application, the lipid composition comprises a plurality of particles with a polydispersity index (PDI) from about 0.1 to about 0.5. In some embodiments of the pharmaceutical composition of the present application, the lipid composition comprises a plurality of particles with a polydispersity index (PDI) from about 0.1 to about 0.3. In some embodiments of the pharmaceutical composition of the present application, the lipid composition comprises a plurality of particles with a polydispersity index (PDI) from about 0.2 to about 0.5. In some embodiments of the pharmaceutical composition of the present application, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present application, the lipid composition comprises a plurality of particles with a negative zeta potential of −5 millivolts (mV) or less. In some embodiments of the pharmaceutical composition of the present application, the lipid composition comprises a plurality of particles with a negative zeta potential of −10 millivolts (mV) or less. In some embodiments of the pharmaceutical composition of the present application, the lipid composition comprises a plurality of particles with a negative zeta potential of −15 millivolts (mV) or less. In some embodiments of the pharmaceutical composition of the present application, the lipid composition comprises a plurality of particles with a negative zeta potential of −20 millivolts (mV) or less. In some embodiments of the pharmaceutical composition of the present application, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present application, the lipid composition comprises a plurality of particles with a negative zeta potential of 15 millivolts (mV) or less. In some embodiments of the pharmaceutical composition of the present application, the lipid composition comprises a plurality of particles with a negative zeta potential of 20 millivolts (mV) or less.

In some embodiments of the pharmaceutical composition of the present application, the lipid composition comprises a plurality of particles with a negative zeta potential of −5 millivolts (mV) or more. In some embodiments of the pharmaceutical composition of the present application, the lipid composition comprises a plurality of particles with a negative zeta potential of −10 millivolts (mV) or more. In some embodiments of the pharmaceutical composition of the present application, the lipid composition comprises a plurality of particles with a negative zeta potential of −15 millivolts (mV) or more. In some embodiments of the pharmaceutical composition of the present application, the lipid composition comprises a plurality of particles with a negative zeta potential of −20 millivolts (mV) or more. In some embodiments of the pharmaceutical composition of the present application, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present application, the lipid composition comprises a plurality of particles with a zeta potential of 15 millivolts (mV) or more. In some embodiments of the pharmaceutical composition of the present application, the lipid composition comprises a plurality of particles with a zeta potential of 20 millivolts (mV) or more.

In some embodiments of the pharmaceutical composition of the present application, the lipid composition has an apparent ionization constant (pKa) outside a range of 6 to 7. In some embodiments of the pharmaceutical composition of the present application, the lipid composition has an apparent pKa of about 8 or higher, about 9 or higher, about 10 or higher, about 11 or higher, about 12 or higher, or about 13 or higher. In some embodiments of the pharmaceutical composition of the present application, the lipid composition has an apparent pKa of about 8 to about 13. In some embodiments of the pharmaceutical composition of the present application, the lipid composition has an apparent pKa of about 8 to about 10. In some embodiments of the pharmaceutical composition of the present application, the lipid composition has an apparent pKa of about 9 to about 11. In some embodiments of the pharmaceutical composition of the present application, the lipid composition has an apparent pKa of about 10 to about 13. In some embodiments of the pharmaceutical composition of the present application, the lipid composition has an apparent pKa of about 8 to about 12. In some embodiments of the pharmaceutical composition of the present application, the lipid composition has an apparent pKa of about 10 to about 12.

### Dosing Level

In another aspect, provided is high-potency dosage form of a therapeutic agent (or prophylactic agent) formulated with an ionizable lipid comprising a chalcogen (e.g., O, S, Se), the dosage form comprising a therapeutic agent (or prophylactic agent) assembled with a lipid composition as described herein.

In some embodiments, the therapeutic agent is present in the dosage form at a dose of about 10, 9, 8, 7, 6, 5, 4, 3, 2, 1.5, 1.0, 0.5, 0.2, 0.1, 0.05, 0.02, 0.01, 0.005, 0.002, or 0.001 milligram per kilogram (mg/kg, or mpk) body weight, or of a range between (inclusive) any two of the foregoing values.

In some embodiments, the therapeutic agent is present in the dosage form at a dose of no more than about 10 milligram per kilogram (mg/kg, or mpk) body weight. In some embodiments, the therapeutic agent is present in the dosage form at a dose of no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1 mg/kg, no more than about 0.5 mg/kg, no more than about 0.2 mg/kg, no more than about 0.1 mg/kg, no more than about 0.05 mg/kg, or no more than about 0.01 mg/kg. In some embodiments, the therapeutic agent is present in the dosage form at a concentration of no more than about 5 milligram per milliliter (mg/mL).

In some embodiments, the therapeutic agent (e.g., proteins, nucleic acids) is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the therapeutic agent is present in the dosage form at a concentration of no more than about 5 milligram per milliliter (mg/mL). In some embodiments, the therapeutic agent is present in the dosage form at a concentration of no more than about 2 milligram per milliliter (mg/mL). In some embodiments, the therapeutic agent is present in the dosage form at a concentration of no more than about 1 milligram per milliliter (mg/mL). In some embodiments, the therapeutic agent is present in the dosage form at a concentration of no more than about 0.5 milligram per milliliter (mg/mL). In some embodiments, the therapeutic agent is present in the dosage form at a concentration of no more than about 0.1 milligram per milliliter (mg/mL).

In some embodiments, the therapeutic agent (e.g., proteins, nucleic acids) is present in the dosage form at a concentration of about 10, 9, 8, 7, 6, 5, 4, 3, 2, 1, 0.5, 0.2, or 0.1 microgram per milliliter (g/mL), or of a range between (inclusive) any two of the foregoing values. In some embodiments, the therapeutic agent is present in the dosage form at a concentration of no more than about 10, no more than about 9, no more than about 8, no more than about 7, no more than about 6, no more than about 5, no more than about 4, no more than about 3, no more than about 2, no more than about 1, no more than about 0.5, no more than about 0.2, no more than about 0.1 microgram per milliliter (g/mL).

Any suitable dosage form can be prepared for delivery, for example, via oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the dosage form can be can even be delivered to the eye or the ear by use of creams, drops, or even injection.

In some embodiments, the administration of a dose of the lipid composition provided here can be repeated. If desired, the effective dose of the active lipid composition can be administered as one, two, three, four, five, six or more doses administered separately at appropriate intervals throughout the course of treatment. In some embodiments, the lipid composition can be administered two or three times daily. In some embodiments, the lipid composition will be administered once daily. In some embodiments, the lipid composition is administered about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, or about every 18 weeks. In some embodiments, the lipid composition is administered about every 1 month, about every 2 months, about every 3 months, about every 4 months, about every 5 months, about every 6 months, about every 7 months, about every 8 months, about every 9 months, about every 10 months, about every 11 months, about every 12 months, about every 13 months, about every 14 months, about every 15 months, about every 16 months, about every 17 months, about every 18 months, about every 2 years, about every 2.5 years, about every 3 years, about every 3.5 years, about every 4 years, about every 4.5 years, or about every 5 years. Any subject in need thereof can be treated with the method of the present application. In some embodiments, the subject has been determined to likely respond to the therapeutic agent. For example, the subject may have, is suffering from, or suspected of having a disease or condition. The therapeutic or prophylactic agent(s) as described elsewhere herein may be effective for providing a therapeutic effect for the subject by a variety of mechanisms, for example, via gene therapy (e.g., requiring repeated administration), altered protein production, (e.g., in vivo) chimeric antigen receptor (CAR) T-cell generation, immuno-oncology, vaccine-based approach, reactivation of tumor suppressors, or other mechanisms.

In some embodiments, the subject has been determined to have a (e.g., missense or nonsense) mutation in a target gene. In some embodiments, the mutation in the target gene is associated with a genetic disease or disorder.

In some embodiments, the subject has been determined to exhibit an aberrant expression or activity of a protein or polynucleotide that corresponds to a target gene. In some embodiments, the aberrant expression or activity of the protein or polynucleotide is associated with a genetic disease or disorder In some embodiments, the subject is selected from the group consisting of mouse, rat, monkey, and human. In some embodiments, the subject is a human.

In another aspect, provided herein is a method for potent delivery of a therapeutic agent (or prophylactic agent) to a cell comprising contacting the cell with the pharmaceutical composition of the present application. In some embodiments of the method, the pharmaceutical composition comprises a therapeutic agent (or prophylactic agent) assembled with a lipid composition as described in the present application, e.g., wherein the lipid composition comprises any of the head or tail groups disclosed herein.

In some embodiments of the method, the cell is isolated from the subject. In some embodiments of the method, the cell is a cell line (e.g., a retinal or sensory hair cell).

In another aspect, provided herein is a method for targeted delivery of a therapeutic agent (or prophylactic agent) to a cell type comprising contacting the cell with the pharmaceutical composition of the present application. In some embodiments of the method, the pharmaceutical composition comprises a therapeutic agent (or prophylactic agent) assembled with a lipid composition as described in the present application, e.g., wherein the lipid composition comprises any of the head or tail groups disclosed herein.

In some embodiments, the contacting is ex vivo. In some embodiments, the contacting is in vitro. In some embodiments, the contacting is in vivo. In some embodiments, the contacting comprises administering to a subject the composition comprising the therapeutic agent assembled with the lipid composition.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkyl-S—.

The term “amide”, as used herein, refers to a group

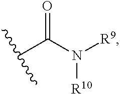

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

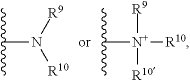

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

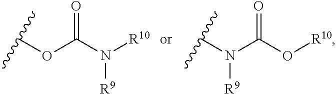

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In some embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In some embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

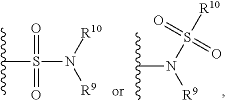

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹, wherein R⁹ represents a hydrocarbyl.

The term “thioether”, as used herein, is equivalent to an ether, wherein the oxygen is replaced with a sulfur.

The term “urea” is art-recognized and may be represented by the general formula

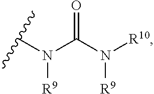

wherein R⁹ and R¹⁰ independently represent hydrogen or a hydrocarbyl.

The term “helper lipid” as used in this disclosure refers to a lipid that contributes to the stability or delivery efficacy of a lipid composition. A helper lipid can be a zwitterionic lipid, such as a phospholipid. A helper lipid can be phosphatidylcholine, distearoylphosphatidylcholine, dioleoylphosphatidylethanolamine, 1,2-dioleoyl-sn-glycero-3-phosphoethanolamine (DOPE) or 1,2-dioleoyl-sn-glycero-3-phosphocholine (DOPC).

The term “modulate” as used herein includes the inhibition or suppression of a function or activity (such as cell proliferation) as well as the enhancement of a function or activity.

The phrase “pharmaceutically acceptable” is art-recognized. In some embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Salt” is used herein to refer to an acid addition salt or a basic addition salt.

Many of the lipidoid compositions (e.g., nanoparticles) useful in the methods and compositions of this disclosure have at least one stereogenic center in their structure. This stereogenic center may be present in a R or a S configuration, the R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers. Some of the lipidoid compositions (e.g., nanoparticles) may also comprise chemical compound which exist in tautomeric forms. Such forms, although not explicitly indicated in the formulae described herein, are intended to be included within the scope of the present disclosure.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The term “pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group that is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁-C₈ alkyl, C₂-C₈ alkenyl, C₂-C₈ alkynyl, aryl, C₇-C₁₂ substituted aryl, and C₇-C₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In some embodiments, the subject is a human. In some embodiments, the subject is a non-human animal. The terms “human,” “patient,” and “subject” are used interchangeably herein.

An “effective amount” means the amount of a compound that, when administered to a subject for treating or preventing a disease, is sufficient to affect such treatment or prevention. The “effective amount” can vary depending on the compound, the disease and its severity, and the age, weight, etc., of the subject to be treated. A “therapeutically effective amount” refers to the effective amount for therapeutic treatment. A “prophylatically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

The term “prophylaxis” is related to “prevention,” and refers to a measure or procedure the purpose of which is to prevent, rather than to treat or cure a disease. Non limiting examples of prophylactic measures may include the administration of vaccines.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In some embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In some embodiments, the weights are based upon total weight of compound.

As used herein and unless otherwise indicated, the term “enantiomerically pure S-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In some embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In some embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In some embodiments, the enantiomerically pure S-compound in such compositions can, for example, comprise, at least about 95% by weight S-compound and at most about 5% by weight R-compound, by total weight of the compound. In some embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

As illustrated through certain embodiments in the examples and the accompanying figures, the disclosure relates to lipid nanoparticles that can be used to deliver RNA (e.g., mRNA) to the eye or ear (e.g., cochlea), for example to treat various conditions.

### Example 1: mRNA Delivery

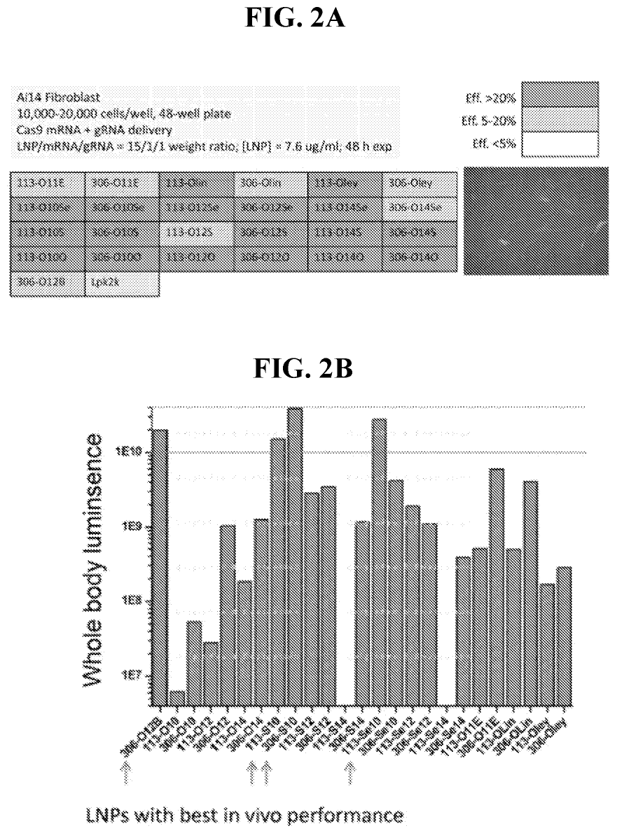

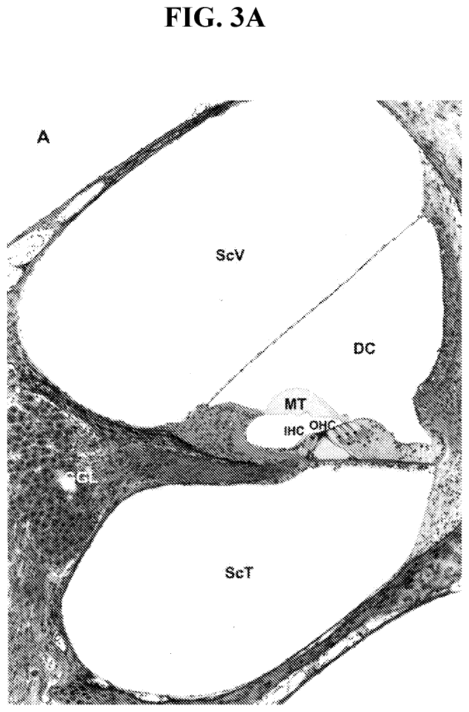

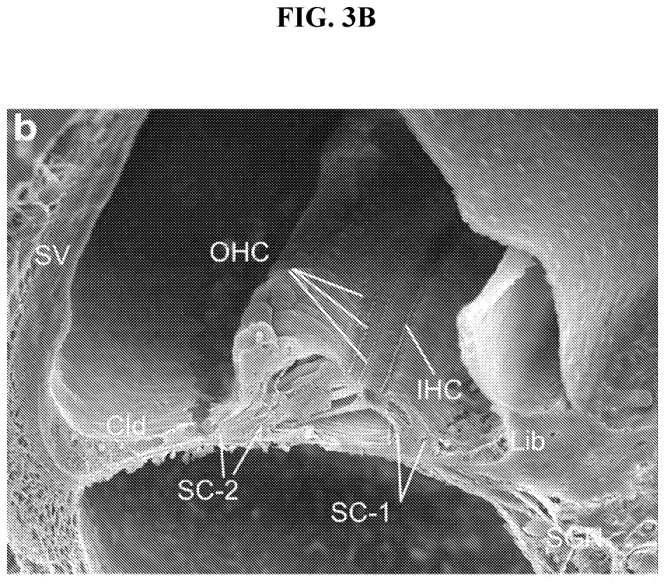

A plurality of LNP compositions were first used to test mRNA delivery in vitro in Ai14 fibroblast cells and in vivo in wildtype mice. Briefly, Cas9 mRNA, LNP, and gRNA were contacted with Ai14 fibroblast cells in wells. The ratio of LNP:mRNA:gRNA delivered to each well was about 15:1:1 weight ratio, where the concentration of LNP was about 7.6 μg/mL. The contents of a well were exposed for about 48 hours. The efficacy of each Cas9 mRNA LNP may be evaluated according to Table 1, as shown in FIG. 2A. In Table 1, the efficacy is categorized as A (>20% efficacy), B (5%<B<20% efficacy), and C (<5% efficacy).

TABLE 1
Efficacy of each Cas9 mRNA LNP

| Cas9 mRNA LNP | In vitro Efficacy* |
|---|---|
| 113-O11E | B |
| 306-O11E | B |
| 113-OLin | A |
| 306-OLin | B |
| 113-OLey | A |
| 306-OLey | B |
| 113-O10Se | A |
| 306-O10Se | A |
| 113-O12Se | A |
| 306-O12Se | A |
| 113-O14Se | A |
| 306-O14Se | B |
| 113-O10S | A |
| 306-O10S | A |
| 113-O12S | B |
| 306-O12S | A |
| 113-O14S | A |
| 306-O14O | A |
| 306-O12B | B |
| Lpk2k | B |

*A (>20% efficacy), B (5% < B < 20% efficacy), and C (<5% efficacy)

Female Balb/c mice (6-8 weeks) were used for in vivo firefly luciferase mRNA (fLuc mRNA, TriLink Biotechnologies) encapsulated LNPs screening and formulations optimization. Briefly, fLuc mRNA LNPs were intravenously injected into the mice at a dose of 10 μg/mouse and imaged for whole body luminescence about 6 hours after injection. fLuc mRNA LNPs may be categorized based on in vivo efficacy (e.g., level of whole-body luminescence measured) after injection of LNPs according to Table 2, as shown in FIG. 2B. In Table 2, the efficacy is categorized as A (>1¹⁰ luminescence), B (1⁸<B<1¹⁰ luminescence), and C (<1⁸ luminescence).

TABLE 2
In vivo efficacy after injection of LNPs

| | In vivo Efficacy (e.g., |
|---|---|
| | whole body |
| fLuc mRNA LNP | luminescence)* |
| 306-O12B | A |
| 113-O10 | C |
| 306-O10 | C |
| 113-O12 | C |
| 306-O12 | B |
| 113-O14 | B |
| 306-O14 | B |
| 113-S10 | A |
| 306-S10 | A |
| 113-S12 | B |
| 306-S12 | B |
| 113-S14 | C |
| 306-S14 | B |
| 113-Se10 | A |
| 306-Se10 | B |
| 113-Se12 | B |
| 306-Se12 | B |
| 113-Se14 | C |
| 306-Se14 | B |
| 113-O11E | B |
| 306-O11E | B |
| 113-OLin | B |
| 306-OLin | B |
| 113-Oley | B |
| 306-Oley | B |

*A (>1¹⁰ luminescence), B (1⁸ < B < 1¹⁰ luminescence), and C (<1⁸ luminescence)

### Example 2; Material and Methods for Delivery into Adult Mouse Cochlea

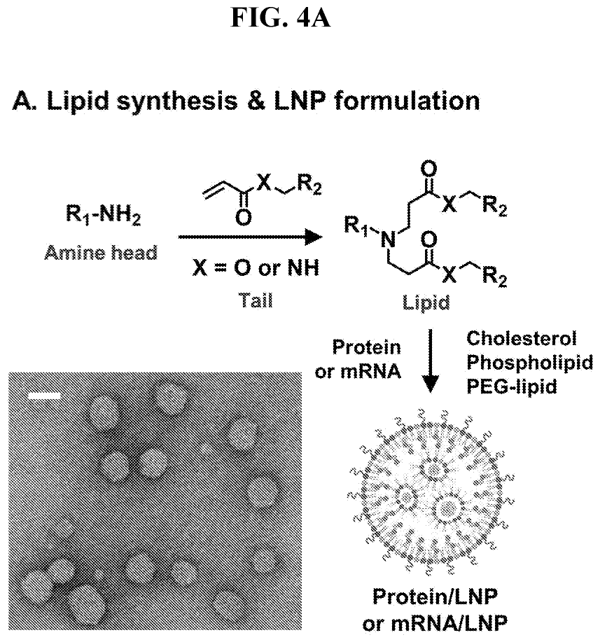

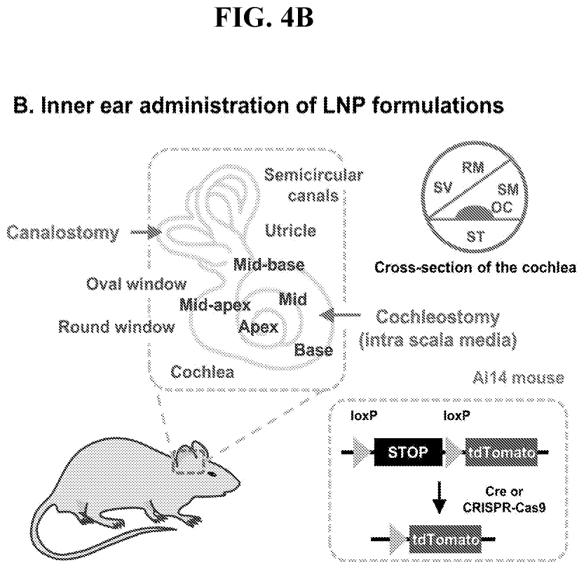

The aim was to develop a new group of LNPs for the delivery of genome engineering Cre and CRISPR-Cas9 systems in the format of protein or mRNA into adult mouse cochlea (FIG. 4A). The adult Ai14 mouse model (congenic on the C57BL/6 genetic background) was used, which harbors a loxP-flanked STOP cassette preventing transcription of a red fluorescent protein, tdTomato (tdT). This mouse model provides a simple readout, as the tdT expression can be triggered by Cre- or CRISPR-Cas9-mediated genome engineering. In this study, the primary focus was on genome engineering events occurring in the cochlea structure (FIG. 4B). As the blood-labyrinthine barrier (BLB) efficiently prevents the entry of substances into the inner ear after systemic administration, the focus was on local administration strategies for the delivery of LNP-complexed genome engineering components, including canalostomy (intra semicircular canals) and cochleostomy (intra scala media). The scala tympani (ST) and scala vestibuli (SV) are filled with perilymph and the scala media (SM) is immersed in endolymph. Protein/LNP or mRNA/LNP complex could directly enter into the perilymph or endolymph in the cochlea after canalostomy or cochleostomy. Through LNP formulation screening and optimization, a series of LNPs were identified that could deliver genome engineering protein and mRNA to the sensory and non-sensory regions in the adult mouse cochlea.

### 1. Synthesis of Ionizable Lipids

Lipid molecules, including the R-O16B, R-N16B, R-O17O, R-O17S, R-O17Se, R-O12B, R-O10S, R-O12Se, and R-O10Se. Nomenclature of LNPs is depicted in FIG. 4A. R stands for amine head number; B stands for disulfide bonds; the N or O after “R—” stands for nitrogen or oxygen in the tail (X═O or NH). Lipids were synthesized and characterized following our previously reported procedures.

2. (−30)GFP-Cre Protein, Cre mRNA, GFP mRNA, and CRISPR-Cas9 mRNA

The supernegatively charged GFP-tagged Cre recombinase, (−30)GFP-Cre was synthesized following our previously reported procedures. Cre, GFP, and SpCas9 mRNA were purchased from TriLink Biotechnologies. Previously reported CRISPR sgRNA sequence targeting the loxP-STOP cassette was used. The sgRNA with chemical modification (2′-O-methyl at three first and last bases; 3′-phosphorothioate bonds between first three and last two bases) was purchased from Synthego.

### 3. Fabrication of LNP Formulations

R-O16B and R-N16B LNPs were fabricated as follows: R-O16B or R-N16B lipids were dissolved in pure ethanol and combined with cholesterol (Sigma-Aldrich), DOPE (1,2-dioleoyl-sn-glycero-3-phosphoethanolamine, Avanti Polar Lipids), DSPE-PEG2k (1,2-distearoyl-sn-glycero-3-phosphoethanolamine-N-[methoxy(polyethylene glycol)-2000], Avanti Polar Lipids) at a 16/4/1/1 weight ratio. The ethanol solution was added dropwise to sodium acetate buffer (25 mM, pH 5.2) with continuous stirring. After dialysis (MWCO 3.5 kDa, Slide-A-Lyzer, ThermoFisher Scientific), the LNPs were mixed with (−30)GFP-Cre in PBS, and the complex was incubated for 30 min at room temperature before use.

R-O17O, R-O17S, and R-O17Se LNPs were fabricated as follows: R-O17O, R-O17S, or R-O17Se lipids combined with cholesterol, DOPE, DSPE-PEG2k (16/4/1/1, weight ratio) were dissolved in pure ethanol and mixed with sodium acetate buffer (25 mM, pH5.2) dissolved with Cre mRNA or GFP mRNA (lipid/mRNA=10/1, weight ratio) using the NanoAssemblr® Ignite microfluidic system. The nanocomplexes were then purified through dialysis (MWCO 3.5 kDa, Slide-A-Lyzer, ThermoFisher Scientific) and stored at 4° C. before use. For the preparation of LNPs with excipients, 76-O17Se/cholesterol/DOPE/DSPE-PEG2k lipid mixture was combined with 80-EC16 (synthesized according to previously reported procedures), (2-hydroxypropyl)-β-cyclodextrin (HP-β-CD; Sigma-Aldrich), or stearic acid (SA; Sigma-Aldrich) in a 1/1 weight ratio (excipient/76-O17Se) and then mixed with mRNA dissolved in sodium acetate buffer using the NanoAssemblr® Ignite microfluidic system. For the preparation of TAT-modified LNPs (TAT-76-O17Se and TAT-78-O17O), 76-O17Se (or 78-O17O)/cholesterol/DOPE/DSPE-PEG2k lipid mixture was combined with DSPE-PEG2k-Maleimide (Avanti Polar Lipid) with a 4/1 molar ratio (76-O17Se or 78-0180/DSPE-PEG2k-Maleimide). After the NanoAssemblr® Ignite microfluidic mixing with cargo mRNA, the cysteine-labeled TAT peptide (MW=1662 Da; GeneScript) was added (cysteine/maleimide=2/1 molar ratio) and incubated at room temperature for 12 h. The nanocomplexes were then purified through dialysis (MWCO 3.5 kDa, Slide-A-Lyzer, ThermoFisher Scientific) and stored at 4° C. before use.

R-O12B, R-O10S, R-O12Se, and R-O10Se LNPs were fabricated as follows: R-O12B, R-O10S, R-O12Se, or R-O10Se lipids combined with cholesterol, DOPC, PEG2k-DMG (16.7/4/2.1/1, weight ratio) were dissolved in pure ethanol and mixed with sodium acetate buffer (25 mM, pH5.2) dissolved with SpCas9 mRNA and sgRNA (lipid/mRNA/sgRNA=15/1/1, weight ratio) using a microfluidic system. The nanocomplexes were then purified through dialysis (MWCO 3.5 kDa, Slide-A-Lyzer, ThermoFisher Scientific) and stored at 4° C. before use.

### 4. Characterization of LNP Formulations

Average hydrodynamic diameter (<Dₕ>) and polydispersity index (PDI) of LNPs were measured by Zeta-PALS particle size analyzer (Brookhaven Instruments). TEM images were taken on an FEI Technai Transmission Electron Microscope. Phosphotungstic acid was used for TEM sample staining.

### 5. In Vitro Delivery of (−30)GFP-Cre/LNPs

HeLa-DsRed cells were seeded in 48 well plates (seeding density=20 k cell/well) and cultured in DMEM medium for 24 h prior to transfection (37° C., 5% CO₂). (−30)GFP-Cre/LNPs formulations were then added. The final concentration of (−30)GFP-Cre protein was 50 nM, and the lipid concentration was 6.6 g/mL. After 8 h of incubation, cells were washed, harvested, and the GFP fluorescence signals were analyzed by flow cytometry (BD FACS Calibur; BD Science).

6. In Vitro Delivery of Cre mRNA/LNPs

Similar to the protein delivery, HeLa-DsRed cells were first seeded in 48 well plates (seeding density=20 k cell/well) and cultured in DMEM medium for 24 h prior to transfection (37° C., 5% CO₂). Cre mRNA/LNPs formulations were then added to each well. The final concentration of Cre mRNA was 0.74 g/mL, and the lipid concentration was 7.4 g/mL. After 24 h of incubation, cells were collected, and the DsRed fluorescence signals were analyzed by flow cytometry (BD FACS Calibur; BD Science).

### 7. Ai14 Mouse Dermal Fibroblast Cell Isolation and Transfection

The dermal fibroblast cells were isolated from Ai14 mice and cultured following previously reported procedures. To transfect the fibroblasts, Cre/LNPs formulations were added and incubated for 48 h before DAPI staining and fluorescence microscope examination (Leica TCS SP5 microscope).

8. Microinjection into the Adult Mouse Inner Ear by Canalostomy and Cochleostomy

All in vivo experiments met the NIH guidelines for the care and use of laboratory animals and were approved by the Massachusetts Eye & Ear IACUC committee. 4-8 week-old adult Ai14 mice and CD-1 mice of either sex were purchased from Jackson Laboratory and randomly assigned to different experimental groups. At least three mice were injected in each group. All surgical procedures were done in a clean, dedicated space. Instruments were thoroughly cleaned with 70% ethanol and autoclaved prior to surgery. Mice were anesthetized by intraperitoneal injection of a combination of xylazine (10 mg/kg body weight) and ketamine (100 mg/kg body weight).

For canalostomy, a 10-mm postauricular incision was made under the operating microscope, and the right pinna and the sternocleidomastoid muscle were extracted to expose the posterior semicircular canal (PSCC) that was located in the margin of temporal bone. A Bonn micro probe was used to drill a small hole on the PSCC, then left it open for a few minutes until no obvious perilymph leakage was observed. The tip of the polyimide tube (ID 0.0039 inches, OD 0.0049 inches, Microlumen) was inserted into the PSCC toward the ampulla. The hole was sealed with tissue adhesive (3M Vetbond, St. Paul, MN), and the lack of fluid leakage indicated the tightness of the sealing. The tubing was cut after injection, with approximately 5 mm of tubing left connected to the PSCC and sealed with tissue adhesive. The volume for each injection was 1 μL per cochlea. The release rate was 169 nL/min, controlled by MICRO4 microinjection controller (WPI). The skin was closed with 5-0 nylon suture (Ethicon Inc., Somerville, NJ). The total surgery time was approximately 20 min, including a 6-min injection period.

Cochleostomy was performed by preauricular incision to expose the cochlear bulla. Anatomic landmarks included the stapedial artery and tympanic ring, which were identified before injection. Glass micropipettes (4878, WPI) were pulled with a micropipette puller (PP83, Narishige) to a final OD of ˜10 μm. Needles held by a Nanoliter 2000 micromanipulator (WPI) were used to manually deliver the LNP formulations into the scala media. The injection site is at the base of the cochlea. The injection volume was 0.3 μL per cochlea. The release rate was 69 nL/min, controlled by MICRO4 microinjection controller (WPI).

### 9. Inner Ear Tissue Dissection and Immunostaining

Injected and non-injected cochleae were harvested after animals were sacrificed by CO2 inhalation. Temporal bones were fixed in 4% paraformaldehyde at 4° C. overnight, then decalcified in 120 mM EDTA at least 1 week. The cochleae were dissected in pieces from the decalcified tissue for whole-mount immunofluorescence. Tissues were infiltrated with 0.3% Triton X-100 and blocked with 8% donkey serum for 1 h before applying the first antibody. 1:500 rabbit anti-MYO7A (#25-6790, Proteus BioSciences), 1:750 chicken anti-GFP (ab13970, Abcam) and 1:350 goat anti-SOX2 (sc-17320, Santa Cruz Biotechnology) were used at room temperature overnight. The second antibody was incubated for 1 h after three rinses with PBS rinses. All Alexafluor secondary antibodies were from Invitrogen: donkey anti-rabbit Alex488 (A21206) or Alex 594 (A21207), donkey anti goat Alex594 (A11058) or Alexa-488-phalloidin (A12379) and goat anti-chicken Alex488 (A-11039) were used as a 1:500 dilution. Specimens were mounted in ProLong Gold Antifade Mountant medium (P36930, Life Technologies). Confocal images were taken with a Leica TCS SP5 microscope using a 20× or 63×glycerin-immersion lens, with or without digital zoom.

### Example 3: (−30)GFP-Cre/LNPs Transfected the Non-Sensory Region of the Cochlea in Adult Ai14 Mouse by Canalostomy

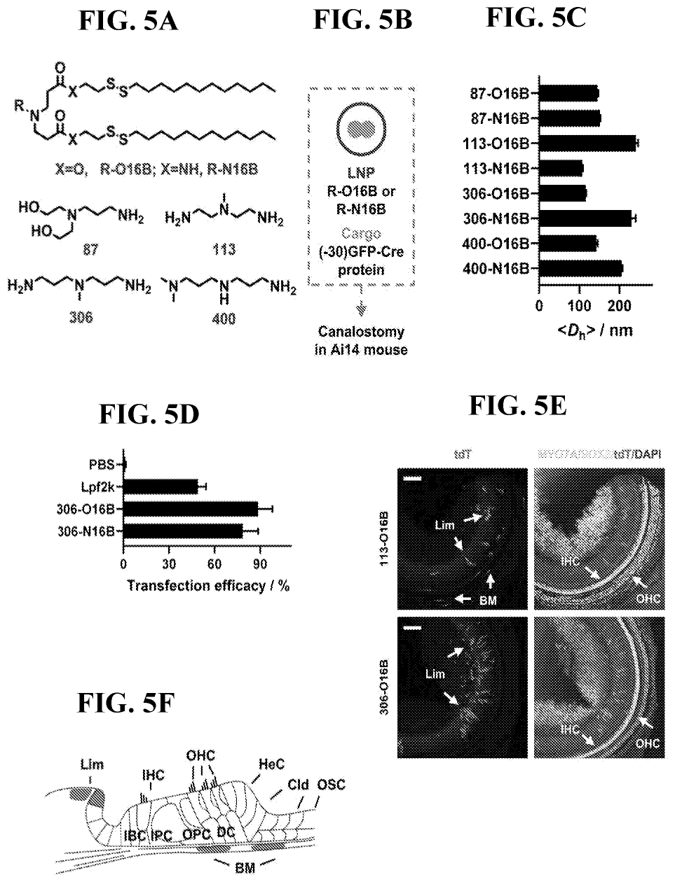

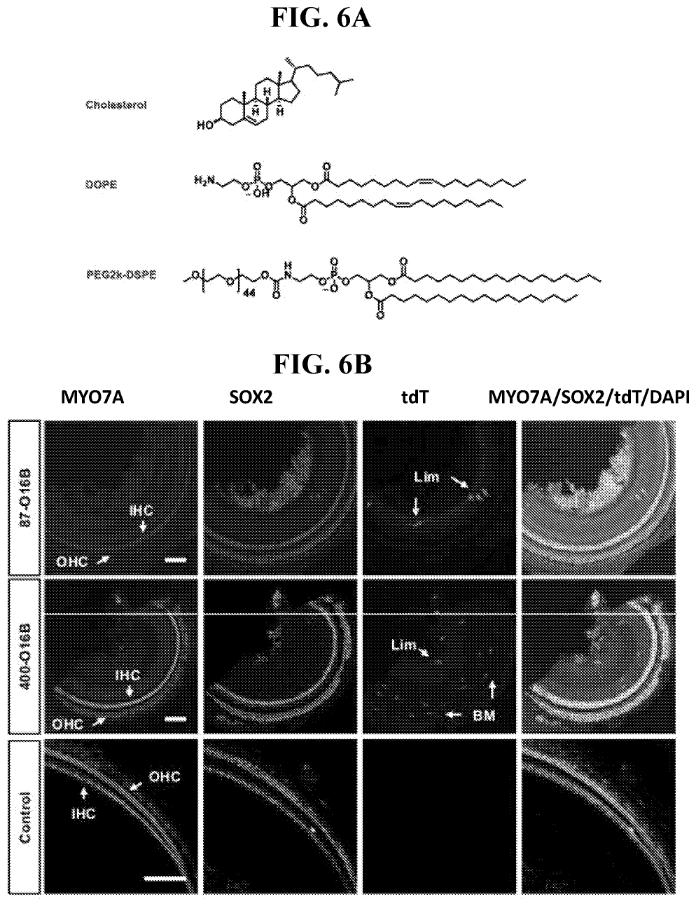

The possibility of using synthetic LNPs for the delivery of (−30)GFP-Cre recombinase protein to the inner ear of adult Ai14 mice through canalostomy was examined. A group of disulfide bond-containing lipids (FIG. 5A) with either ester linkage (R-O16B; R represents amine head number; i.e. 87-O16B, 113-O16B, 306-O16B, and 400-O16B) or amide linkage (R-N16B; i.e. 87-N16B, 113-N16B, 306-N16B, and 400-N16B) were selected to fabricate LNP formulations with cholesterol, DOPE, and DSPE-PEG2k (R-O16B or R-N16B/cholesterol/DOPE/DSPE-PEG2k=16//4/1/1, weight ratio; FIG. 6A). In a previous study, the O16B and N16B-based bioreducible LNPs were demonstrated to be effective for intracellular delivery of (−30)GFP-Cre protein in vitro in cultured cell line. In this study, the (−30)GFP-Cre protein-loaded R-O16B/R-N16B LNP formulations ((−30)GFP-Cre/LNPs) were tested for in vivo genome engineering in the inner ear of adult Ai14 mice through canalostomy (FIG. 5B).

The (−30)GFP-Cre/LNPs formulations were fabricated following previously reported procedures and characterized by dynamic light scattering (DLS), and all LNPs showed average hydrodynamic diameter (<Dₕ>) in the range of 100-250 nm (FIG. 5C), with the 113-N16B (107.6±1.4 nm) being the smallest and 113-O16B (240.9±5.3 nm) being the biggest. The sizes of these LNP formulations are in the optimal range for intracellular delivery applications. Next, the transfection efficacies (indicted by GFP-positive cell percentage) of (−30)GFP-Cre loaded 306-O16B and 306-N16B LNPs were investigated by co-incubating the LNP formulations with HeLa-DsRed cells for 8 h. Flow cytometry analysis revealed that 306-O16B (88.5±9.5%) and 306-N16B (78.5±10.2%) LNPs could efficiently deliver (−30)GFP-Cre protein into culture cells, and they outperformed the positive control, Lpf2k (48.9±5.5%; FIG. 5D). The rest of the LNPs, including 87-, 113-, and 400-O16B, and 87-, 113-, and 400-N16B, were all highly active (induced 80-90% transection efficacies) in HeLa-DsRed cells as described in a previous report.

The (−30)GFP-Cre/LNPs formulations were then injected into adult Ai14 mouse inner ear through canalostomy. 7-8 days post-injection, cochlea was dissected, processed, and immune-stained for fluorescence imaging. It was found that the O16B-based LNPs all induced successful Cre-mediated genome recombination as indicated by the tdT-positive signals observed in the cochlea structure (FIG. 5E), and the 113-, 306-, and 400-O16B LNPs induced a larger number of tdT-positive cells than the 87-O16B (FIG. 6B). Further analysis revealed that the tdT-positive cells were mainly located at the basilar membrane (BM) and limbus (Lim) of the cochlea (FIG. 5F). The BM is an important component in the cochlea, as it provides the foundation for the OC (FIG. 4B), separates the endolymph in the SM from the perilymph in the ST, and disperses and transforms sound waves. The Lim is also critical for maintaining the normal structure and function of the cochlea. Thus LNP-assisted protein delivery through canalostomy could induce successful genome engineering events in the cells of BM and Lim in adult mice.

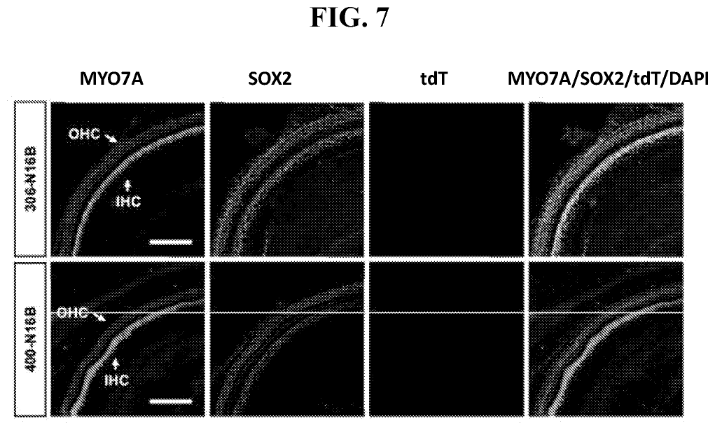

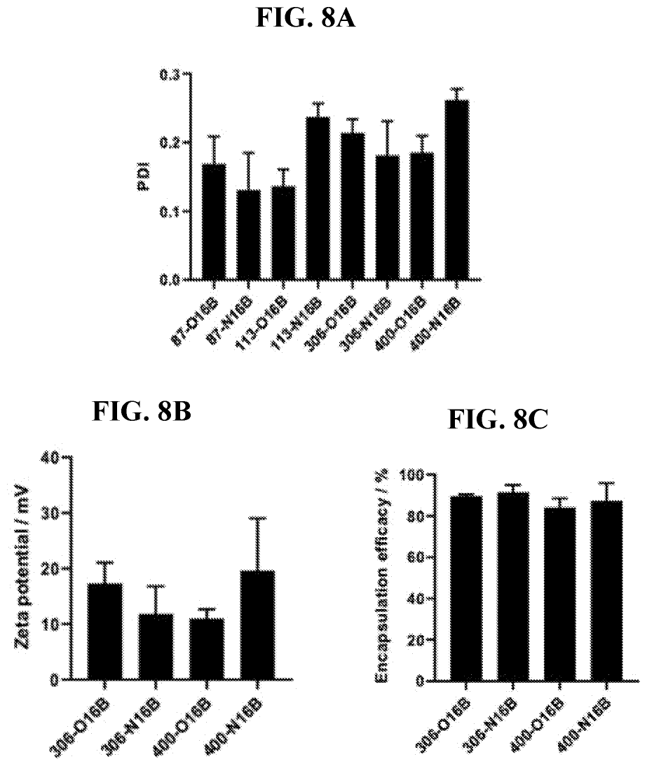

It was also noticed that, under the same tested conditions, injection of the N16B-based LNPs did not result in any tdT signals in the cochlea (FIG. 7). Even though both O16B- and N16B-based LNPs transfected HeLa-DsRed cells in vitro with high and comparable efficacies, these two types of LNPs behaved very differently in vivo in the mouse inner ear. The physicochemical properties of the O16B- and N16B-based LNPs were examined. The polydispersity indexes (PDIs) of all tested LNPs are in the range of 0.13-0.26, indicating the hydrodynamic size uniformity of these LNPs (FIG. 8A). No obvious differences between O16B- and N16B-based LNPs were observed. Both the O16B- and N16B-based blank LNPs were positively charged, as indicated by the positive zeta-potential values (FIG. 8B). After complexing with (−30)GFP-Cre, the cargo protein encapsulation efficacies of LNPs were determined to be 78.5-90.5%, and the N16B-based LNPs did not show significant lower encapsulation efficacy than the O16B-based LNPs (FIG. 8C). Recent study showed that the differences in serum protein corona formed on the surface of bioreducible LNPs (012B- and N12B-based LNPs) after systemic administration could result in different in vivo distribution profiles and delivery performance. Similarly, it is reasonable to speculate that the differences in surface chemistry of O16B- and N16B-based LNPs may play a role in determining their biological fate in the inner ear through interacting with proteins and other types of biomolecules in the perilymph after canalostomy. The structure-activity relationship study of LNPs for inner ear delivery is underway in our lab.

### Example 4: Cre mRNA/LNPs Transfected the Non-Sensory Region of the Cochlea in Adult Ai14 Mouse by Canalostomy

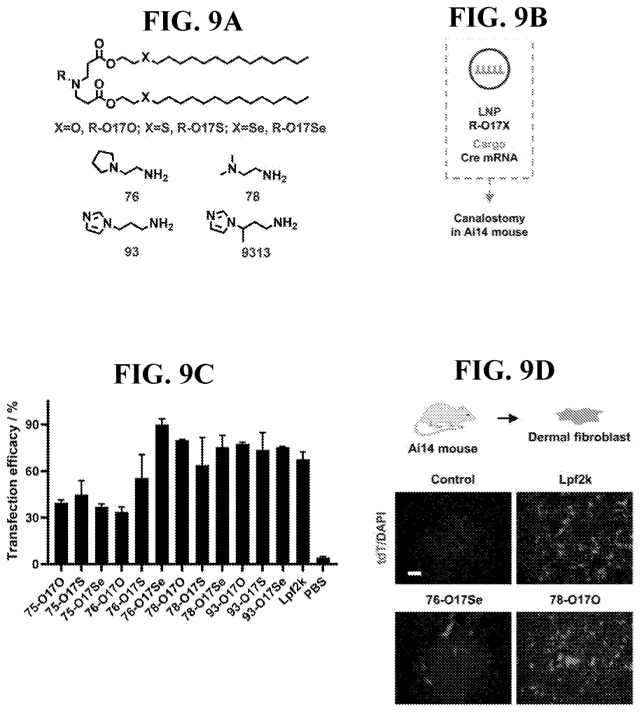

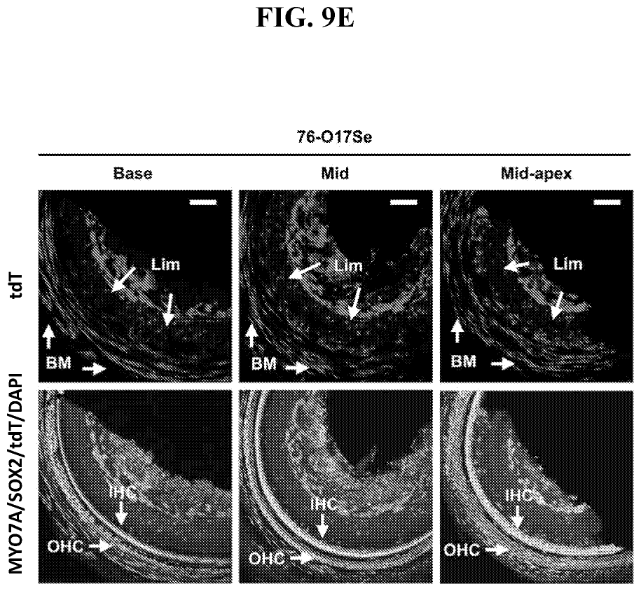

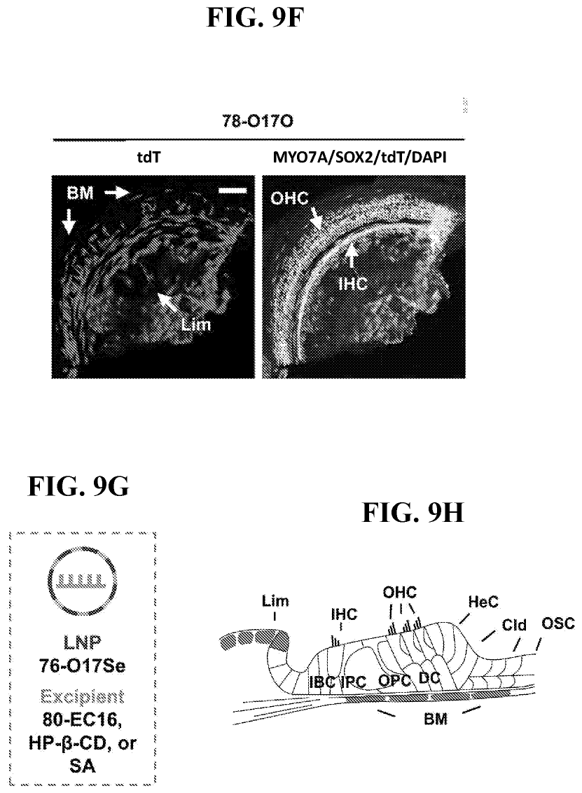

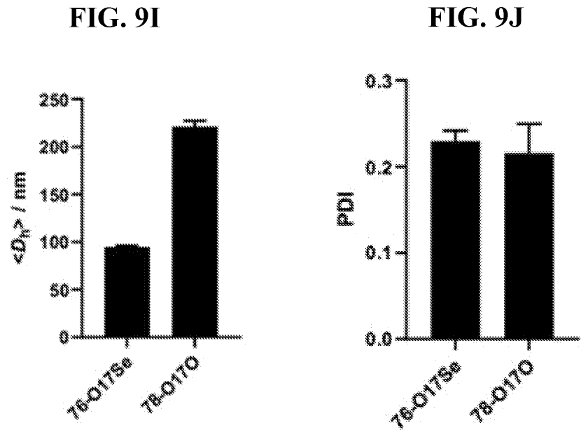

Besides protein delivery, the possibility of LNP-assisted mRNA delivery in the inner ear for genome engineering was explored. In previous studies, a library of chalcogen-containing LNPs were developed, named R-O17X (X═O, S, or Se; lipid tails with ether, thioether, or selenide ether bonds). Several R-O17X LNPs were identified to be effective for protein or mRNA delivery both in vitro and in vivo in adult mice after systemic administration. A small group of R-O17X LNPs, including 75-, 76-, 78-, and 93-O17X (e.g. 75-O17O, 75-O17S, and 75-O17Se; FIG. 9A) were selected and investigated the R-O17X LNP-mediated Cre mRNA delivery in the inner ear of adult Ai14 mice through canalostomy (FIG. 9B). The R-O17X lipids, cholesterol, DOPE, and DSPE-PEG2k (R-O17X/cholesterol/DOPE/DSPE-PEG2k=16//4/1/1, weight ratio) were used to fabricate LNPs and encapsulate Cre mRNA through a microfluidic-assisted self-assembly procedure.

To test if the Cre mRNA-loaded R-O17X LNPs (Cre mRNA/LNPs) can deliver the cargo mRNA in vitro and induce successful gene combination events, Cre mRNA/LNPs were incubated with HeLa-DsRed cells, which express DsRed fluorescent protein upon Cre-mediated loxP-STOP cassette removal. Cre mRNA/Lpf2k- and PBS-treated cells were used as positive and negative controls, respectively. After 24 h of incubation, cells were collected and the LNPs transfection efficacies (represented by DsRed-positive cell percentage) were quantified using flow cytometry (FIG. 9C). Compared to Lpf2k, which showed 67.6±4.7% efficacy, the 75-O17X (i.e. 75-O17O, 75-O17S, and 75-O17Se), 76-O17O, and 76-O17S LNPs were less efficient, as 33-56% transfection efficacies were achieved. However, the rest of the R-O17X LNPs all had comparable or even higher transfection efficacies (63-91%) than the Lpf2k. This corroborated with our previous studies that the chalcogen-containing LNPs were highly active in intracellular delivery of biomacromolecules including proteins and mRNA.

As indicated by the in vitro and in vivo results of the disulfide bond-containing LNPs (FIGS. 5A to 5F), the HeLa-DsRed cell line may not be optimal for predicting transfection performance in the mouse inner ear. The adult Ai14 dermal fibroblasts were then isolated, and two of the most effective LNP formulations identified from the HeLa-DsRed transfection study, i.e. 76-O17Se and 78-O17O, were tested (FIG. 9D). These two LNPs were characterized at first. DLS measurements showed that 76-O17Se and 78-O17O LNPs had average hydrodynamic sizes of 94.6±1.4 and 221.3±6.2 nm, respectively (FIGS. 91 and 9J). Both LNPs are with uniform size distributions as indicated by the PDI values of ˜0.2. After 48 h of incubation with adult Ai14 dermal fibroblasts, both 76-O17Se and 78-O17O LNP formulations resulted in positive tdT signals. 78-O17O induced comparable gene recombination efficacy as the positive control, Lpf2k; however, 76-O17Se was found to be less efficient. The differences in LNP delivery efficacy in different types of cells (e.g., HeLa-DsRed and Ai14 dermal fibroblasts) are believed to be associated with the physicochemical property of distinct cell types, which has been commonly observed in previous studies.

Cre mRNA-loaded 76-O17Se and 78-O17O LNPs were injected into the inner ear of adult Ai14 mice through canalostomy. Strong tdT expression from both LNPs was observed in the cochlea structure 7-8 days after administration (FIGS. 9E and 9F). Compared with R-O16B LNP-mediated (−30)GFP-Cre protein delivery (FIG. 5E), the R-O17X-mediated Cre mRNA induced overall much stronger tdT signals. Despite the fact that the protein/LNPs and mRNA/LNPs may interact with inner ear cells differently, the mRNA molecules that are successfully delivered into the cytoplasm can potentially generate multiple copies of protein through translation. Moreover, the tdT signals were observed throughout the whole cochlea structure after mRNA delivery, from the base to the middle and apex regions (FIG. 9E). It was reported that due to the high viscosity of the endolymph in the SM and perilymph in the SV and ST, locally administered drugs oftentimes have very limited diffusion capacity in the cochlea. This seems not to be an issue with the Cre mRNA/LNP formulations tested in here. In fact, due to their high structural flexibility and biological activity, the LNP-based delivery systems have been intensively explored to overcome the viscous mucus physical barriers in the lung, gastrointestinal tract, and cervix.

As to the transfected cell populations, 76-O17Se and 78-O17O showed similar patterns. Similar to protein delivery, Cre mRNA-loaded 76-O17Se and 78-O17O LNPs transfected cells are mainly located in the BM and Lim non-sensory regions. Recent studies showed that the incorporation of neutral, cationic, or anionic small molecular excipients could be useful for tuning the in vivo tissue and cell specificity of LNP formulation. A group of 76-O17Se-based LNP formulations were fabriced and combined with excipients (FIG. 9G), including the tertiary amine-containing lipid 80-EC16, carboxyl group-containing stearic acid (SA), and neutral hydroxyl group-containing (2-hydroxypropyl)-β-cyclodextrin (HP-j-CD; excipient/76-O17Se=1/1, weight ratio; FIG. 10). After canalostomy, all these LNP formulations induced tdT positive signals in the adult Ai14 mouse cochlea (FIG. 11). However, the majority of cells that underwent gene recombination were still in the BM and Lim regions. The 76-O17Se/SA formulation was less efficient compared with others, and strong signals were observed only in the Lim region. It is possible that the incorporation of anionic SA reduced the overall positive surface charge of the LNPs and affected the delivery.

Overall, in spite of the chemical structure differences between disulfide bond-containing R-O16B LNPs and chalcogen-containing R-O17X LNPs and the surface property differences between 76-O17Se LNP and a series of excipient/76-O17Se LNPs, most of the protein- or mRNA-mediated gene recombination events observed so far occurred in the BM and Lim regions. IT was speculated that this phenomenon is related to the administration method that was employed. After the canalostomy, the LNP formulations could migrate from the semicircular canal to the ST of the cochlea through passive diffusion within the perilymph. The BM then had the chance to interact with the LNPs directly and get transfected. In order to deliver the cargo protein or mRNA into Lim cells, the LNPs need to either go from the ST and penetrate through the BM or diffuse into SV and penetrate through the RM. It is unclear right now how the LNPs were trafficked inside of the cochlea which merits further investigation.

### Example 5: GFP mRNA/LNPs Transfected the Sensory Region of the Cochlea in Adult CD-1 Mouse by Cochleostomy

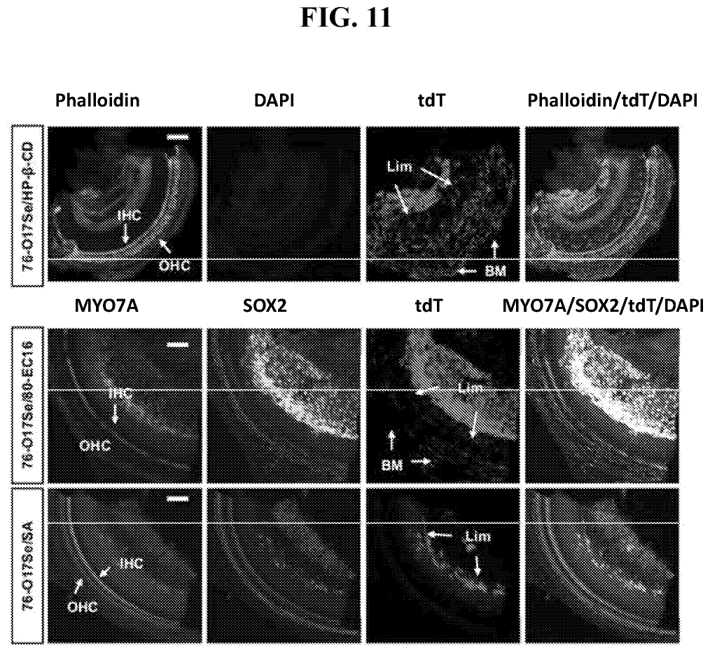

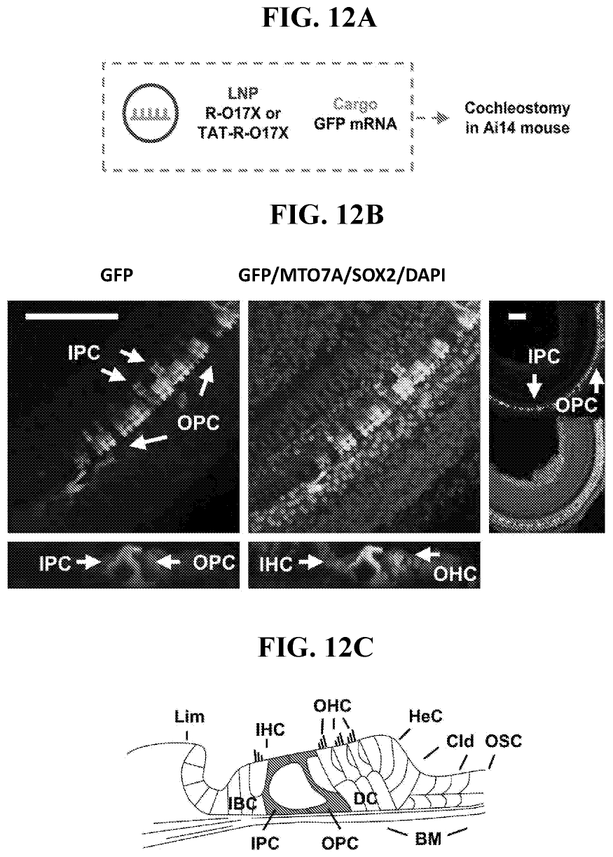

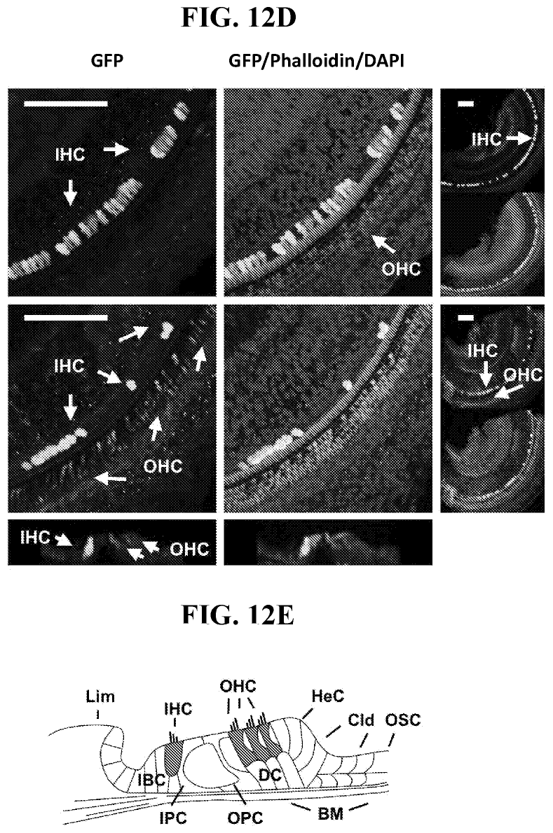

The cells in the BM and Lim have been shown to be readily accessible by LNP formulations through canalostomy, despite the properties of cargo and carrier systems. Cochleostomy (intra scala media injection) was then tested as the administration route for mRNA/R-O17X LNP formulations. Compared with canalostomy, in which LNPs were injected into the perilymph, cochleostomy introduces LNPs directly into the endolymph in the SV. The LNP formulations would be directly exposed to the OC without penetrating RM or BM, so the sensory cells and many other types of cells may have a better chance to be transfected. In this experiment, GFP mRNA was used as the cargo and adult CD-1 mouse model. 76-O17Se and 78-O17O were selected as they were highly effective for mRNA delivery through canalostomy. Using previously described surgical procedures, GFP mRNA-loaded 76-O17Se and 78-O17O LNPs were injected into adult Ai14 mouse inner ear through cochleostomy, and GFP signals in the cochlea were analyzed 3 days post-injection (FIG. 12A).

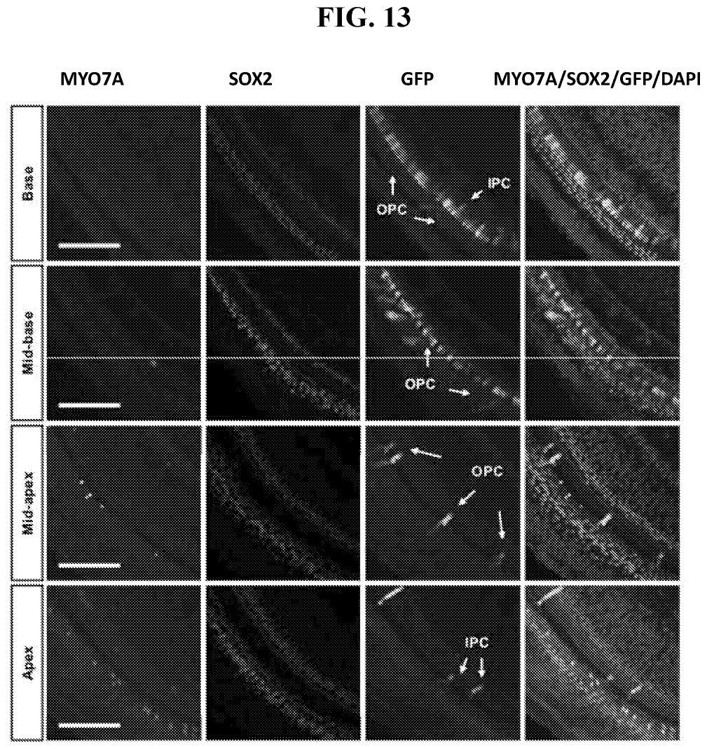

Surprisingly, strong GFP signals were observed right in the middle of the inner hair cells (IHCs) and outer hair cells (OHCs) in the OC of cochlea treated with mRNA/78-O17O LNPs (FIG. 12B). The cross-section image analysis further verified that the GFP-positive cells were inner pillar cells (IPCs) and outer pillar cells (OPCs; FIG. 12C). Furthermore, the 78-O17O transfected and GFP expressing pillar cells were observed throughout the cochlea, from the base (injection site) to mid-base, mid, mid-apex, and apex regions (FIG. 13). The pillar cells are a group of important supporting cells, which provide structural support for the mechanical stimulation of sensory hair cells. Maintaining the morphology and functions of pillar cells is critical for normal hearing, and recent studies have also shown the potential of transforming pillar cells into hair cells through gene therapy, which could be a promising strategy for hearing restoration after hair cell damage and loss. Furthermore, contrary to the canalostomy results, no obvious signals from BM or Lim regions were observed after cochleostomy of GFP mRNA/78-O17O. Under the same tested conditions, the GFP mRNA/76-O17Se LNPs did not produce evident GFP-expressing cells in the cochlea. These results suggested that the administration approach affects the bioactivity of mRNA/LNP formulations in the inner ear.

Further analysis revealed that, unlike the Cre mRNA/76-O17Se LNP-induced tdT signals in Ai14 mice, in which constant tdT fluorescence signal intensity with moderate variations was recorded from the base to mid and apex regions (FIG. 9E), the GFP mRNA/78O17O LNPs resulted in strongest fluorescent protein production near the injection site (base region), and the positive signal intensity decreased gradually from the base to apex. This is likely due to the limited diffusion efficiency of mRNA/78-O17O LNP formulation in the endolymph of SM.

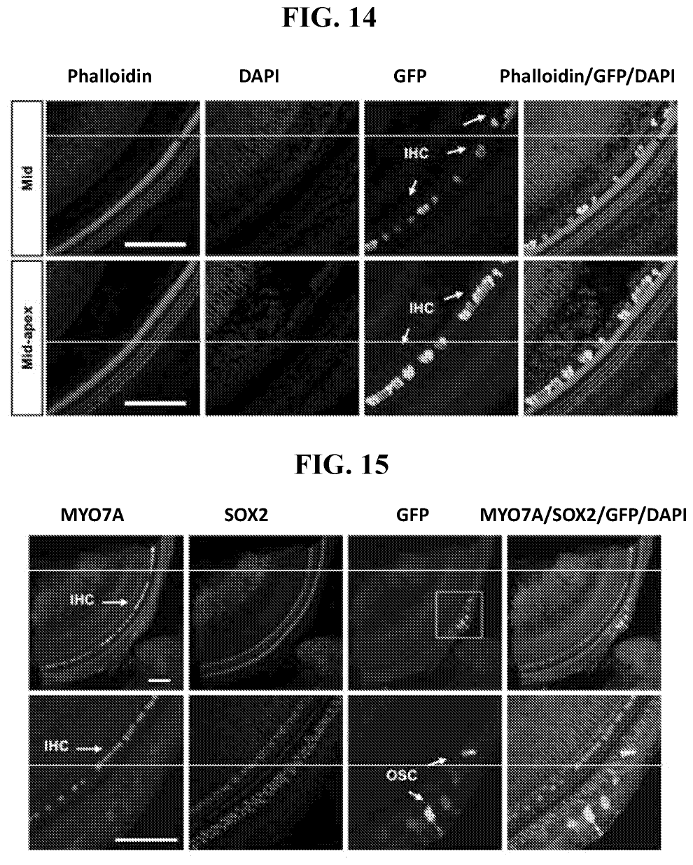

78-O17O LNPs were then modified with the cell-penetrating peptide, TAT, by conjugating cysteine-TAT to the maleimide-PEG2k-DSPE-incorporated 78-O17O LNPs, to see if TAT can facilitate cellular internalization and improve delivery performance. The TAT-78-O17O LNPs delivered GFP mRNA and induced protein expression exclusively in the sensory hair cells (FIG. 12D). Strong signals were observed in the mid, mid-apex, and apex regions. In the mid and mid-apex, GFP signals were mainly recorded in inner hair cells (IHCs; FIG. 14), and in the apex region, both IHCs and outer hair cells (OHCs) expressed the GFP. The GFP signals from OHCs were much weaker than those from IHCs, indicating that the TAT-78-O17O LNPs might be more active in transfecting IHCs. The hair cells are the most vital part of hearing initiation, mechanical signal conversion, and electrochemical signal transduction. A selective and effective delivery system that can transfect hair cells with drugs and nucleic acids can greatly facilitate the development of therapeutics for hearing disorders, and our TAT-labeled 78-O17O LNP system provided a promising platform for further development (FIG. 12E).

As to why TAT modification redirected the 78-O17O LNPs to hair cells, instead of simply enhancing cellular uptake by pillar cells, the underlying mechanism is unclear. It was speculated that the TAT peptide either altered the physicochemical properties of 78-O17O LNPs and/or it can directly interact with certain types of membrane-associated proteins or some other types of biomolecules specifically presented on the hair cells. In addition, 76-O17Se LNPs were modified with TAT peptide (TAT-76-O17Se) and tested it in CD-1 mouse through cochleostomy. Only a small number of outer sulcus cells (OSCs) located in the base region near the injection site was found to be GFP-positive (FIG. 15). This corroborated with the results discussed above that the TAT modification altered the bioactivity of 76-O17Se LNP in the cochlea, as the unmodified 76-O17Se failed to deliver GFP mRNA.

Taken together, these results indicated that, both sensory cells and supporting cells in the OC can be transfected by GFP mRNA/LNPs through cochleostomy. Different administration methods (i.e., canalostomy and cochleostomy) can remarkably impact LNP delivery performance in the inner ear. LNP modification with TAT peptide can alter the cell specificity, while the TAT-induced LNP structural and property changes merit further investigation. These results also suggested that other types of bioactive molecules, peptides, proteins, aptamers, etc. may also be explored for directing the mRNA/LNP formulations to sensory and non-sensory cell populations in the cochlea for cell-specific mRNA delivery.

### Example 6: Cre mRNA/LNPs Transfected the Sensory Region of the Cochlea in Adult Ai14 Mouse by Cochleostomy

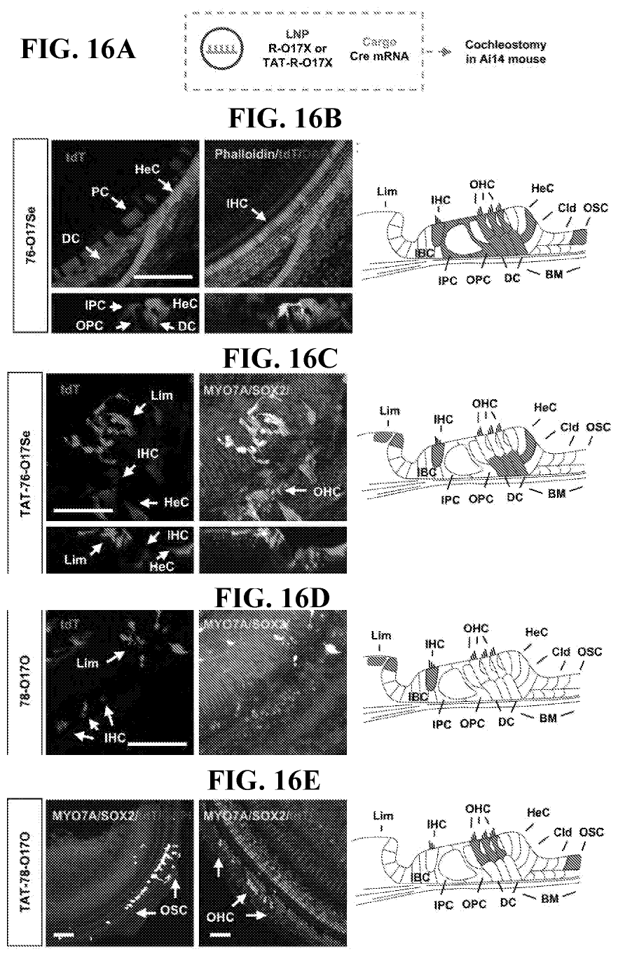

As the GFP mRNA delivery and protein expression in the cochlea sensory region were successfully induced by R-O17X LNPs in adult CD-1 mice through cochleostomy, it was then tested if Cre mRNA can be delivered in adult Ai14 mouse cochlea for gene recombination. Both the unmodified and TAT-modified 76-O17Se and 78-O17O LNPs were investigated (FIG. 16A).

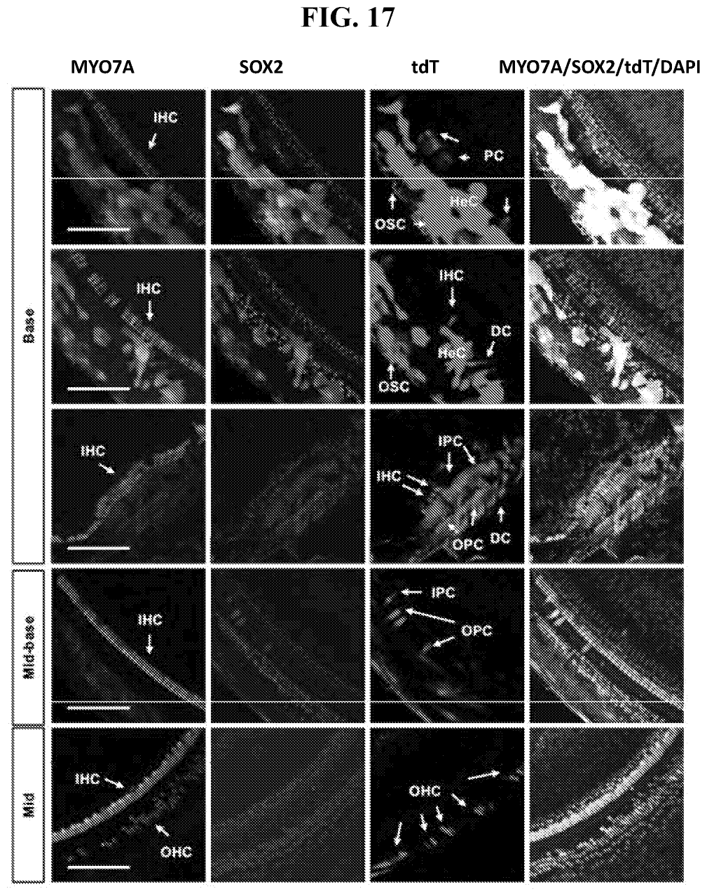

Cre mRNA/76-O17Se LNP formulation treated cochlea of adult Ai14 mice showed tdT positive cells mainly in the base region, which was close to the injection site (FIG. 16B). Contrary to the GFP mRNA/76-O17Se in CD-1 mice, which did not induce any GFP-positive signals, the Cre mRNA/76-O17Se LNP induced strong signals in Ai14 mice after cochleostomy. Although the differences in mouse background should be considered, it was hypothesized that the chemical composition, secondary structure, and/or chain length of the cargo mRNA might affect the biological properties of the mRNA/LNP formulations. This point will be touched upon in the following sections. Furthermore, compared with tdT signals in the base region, a small number of tdT-expressing cells were observed in the mid-base and mid regions (FIG. 17). Similar to the GFP mRNA/78-O17O LNPs (FIG. 13), this phenomenon is likely associated with the low diffusion efficiency of Cre mRNA/76-O17Se LNP in the endolymph of SM. Moreover, Cre mRNA/76-O17Se LNP transfected the BM and Lim regions throughout the whole cochlea from base to apex through canalostomy (FIG. 9E), which suggested that the mRNA/LNP formulations may have different passive diffusion efficiencies in the perilymph and endolymph, in which the differences in chemical composition can play an important role. Further analysis revealed that several types of cells in the OC underwent Cre-mediated gene recombination with the treatment of Cre mRNA/76-O17Se LNPs, including the IPCs, OPCs, IHCs, OHCs, Deiter cells (DCs), Hensen cells (HeCs), and OSCs (FIG. 16B). This pattern is very different from the results obtained from canalostomy, in which cells in the BM and Lim regions were transfected (FIG. 9H). Overall, it was evident that the Cre mRNA/76-O17Se LNPs transfected a group of cells in the sensory region of OC; however, this formulation lacks cell-specificity. The formulation needs further improvement if high specificity is required; however, the current formulation could also be useful when multiple types of cells in the OC need to be transfected.

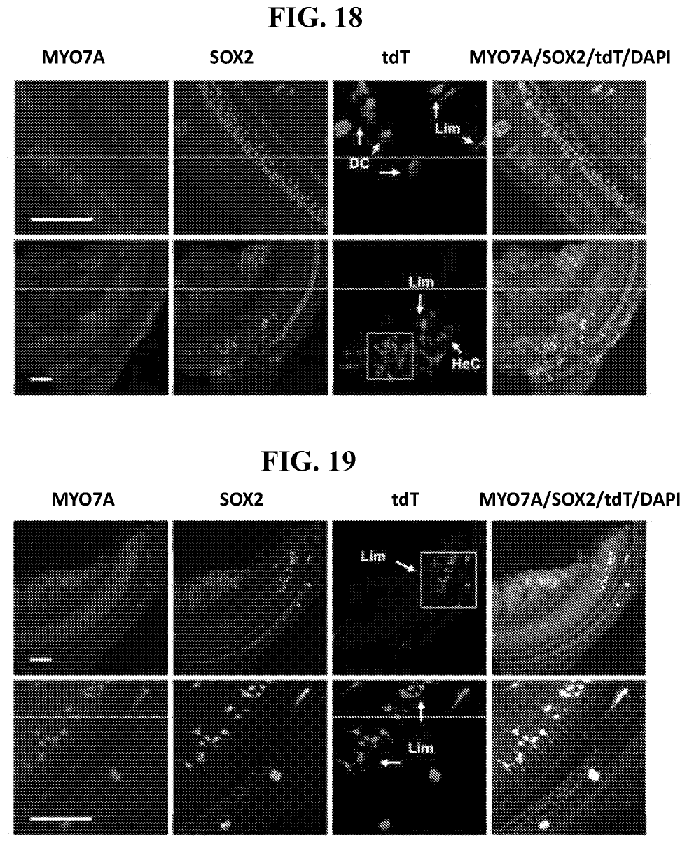

TAT-modified Cre mRNA/TAT-76-O17Se LNPs also produced tdT signals in the OC of adult Ai14 mice (FIG. 16C and FIG. 18). Similar to the unmodified 76-O17Se LNPs, the Cre mRNA-loaded TAT-76-O17Se LNPs produced a larger number of transfected cochlea cells compared with the GFP mRNA-loaded LNPs (FIG. 15). Furthermore, the GFP-positive cells in CD-1 mice were mainly OSCs, while the Cre-mediated tdT-positive cells in Ai14 mice were identified as cells in Lim, DCs, and HeCs. It should be noted that very weak tdT signals were also observed in the IHCs (cross-section images shown in FIG. 16C). Consistent with the results of 76-O17Se LNPs, TAT-76-O17Se LNPs loaded with different mRNA molecules (i.e., GFP or Cre mRNA) showed very different efficacy and cell selectivity in CD-1 and Ai14 mice. In addition, the unmodified and TAT-modified 76-O17Se LNPs produced different patterns of transfected cells (FIG. 16C). Consistent with the results in CD-1 mice, the TAT modification did not simply alter the cellular internalization efficacy of 76-O17Se LNPs, it affected the LNP's affinity to different cell types in the cochlea. Lastly, the TAT-76-O17Se LNP-induced tdT signals were mainly observed in the base region, and negligible signals were found in the mid or apex regions. It suggested that the passive diffusion of TAT-76-O17Se LNP in endolymph of SM is also limited, which is consistent with the results of unmodified 76-O17Se LNPs (FIG. 17).

Cre mRNA-loaded 78-O17O and TAT-78-O17O were also tested in adult Ai14 mice. After administration through cochleostomy, a small number of tdT-positive cells were observed in the base region of 78-O17O LNP-treated cochlea (FIG. 16D and FIG. 19), which were identified as IHCs and cells in the Lim (FIG. 16D). Similarly, a few cochlea cells underwent Cre-mediated recombination in the base and mid regions after TAT-78-O17O LNP administration, which are mainly OSCs and OHCs (FIG. 16E). Clearly, both LNPs loaded with Cre mRNA showed reduced efficacy and different cell affinity in Ai14 mice compared with GFP mRNA-loaded LNPs in CD-1 mice (FIGS. 12A to 12E). It was confirmed that the same type of LNPs loaded with different types of cargo mRNA behaved differently in mice with different genetic backgrounds. The TAT peptide modification also affected the performance of 78-O17O, which was consistent with the results of GFP mRNA delivery in CD-1 mouse cochlea (FIGS. 12C and 12E), as well as the results of Cre mRNA delivery of modified and unmodified 76-O17Se LNPs (FIGS. 16B and 16C). By comparing the canalostomy and cochleostomy, cochleostomy of Cre-loaded 78-O17O and TAT-78-O17O omitted transfection in the BM cells (FIG. 9H), and gene recombination events were observed in the sensory region of OC. These findings are consistent with the results obtained from the modified and unmodified 76-O17Se LNPs as discussed above.

### Example 7: CRISPR-Cas9 mRNA/LNPs Transfected the Sensory Region of the Cochlea in Adult Ai14 Mouse by Cochleostomy

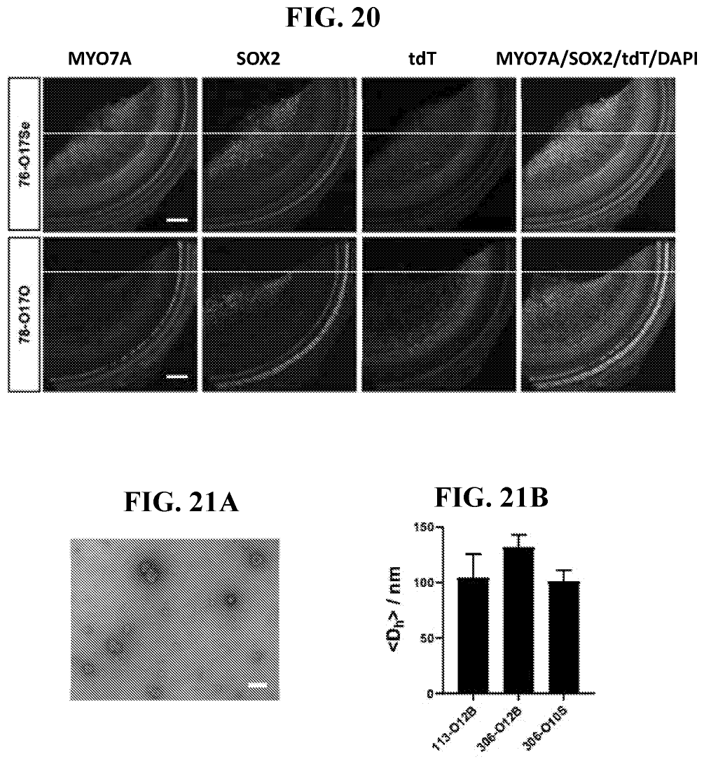

As multiple types of cells in the cochlea could be transfected by Cre mRNA-loaded R-O17X LNPs, it was then tested if R-O17X LNPs could deliver Cas9 mRNA and sgRNA to the cochlea and enable CRISPR-mediated genome engineering. Using a microfluidic-assisted self-assembly approach, SpCas9 mRNA and a sgRNA targeting the loxP-STOP cassette were encapsulated into 76-O17Se and 78-O17O LNPs (lipid/mRNA/sgRNA=15/1/1, weight ratio). The Cas9 mRNA-sgRNA/LNPs were then injected into adult Ai14 mouse cochlea through cochleostomy. However, both 76-O17Se and 78-O17O LNPs failed to induce tdT signals in the cochlea (FIG. 20). It was clear that the Cre mRNA-loaded and Cas9-sgRNA-loaded R-O17X (i.e 76-O17Se and 78-O17O) LNPs had different activity in the cochlea of adult Ai14 mice after cochleostomy administration. As the two R-O17X LNPs were inefficient, it was then explored if the possibility of using other types of synthetic LNPs for Cas9 mRNA-sgRNA delivery in the cochlea was possible.

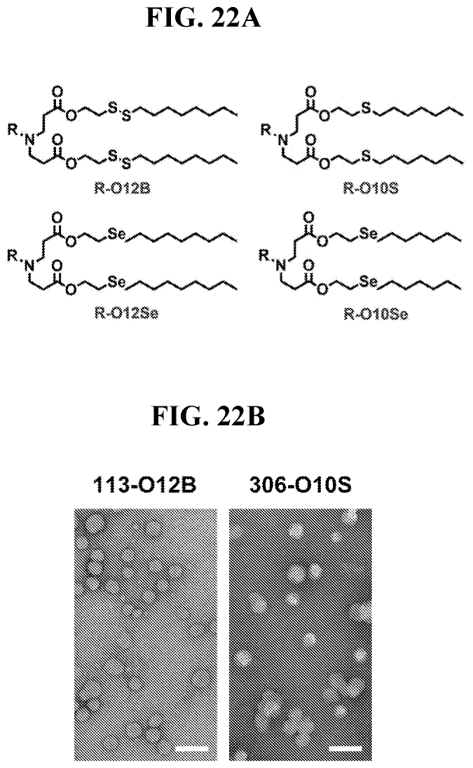

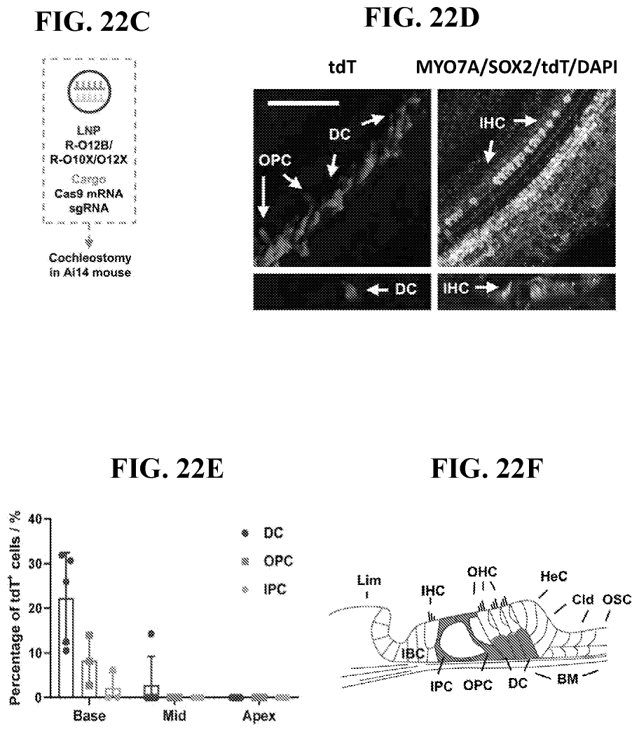

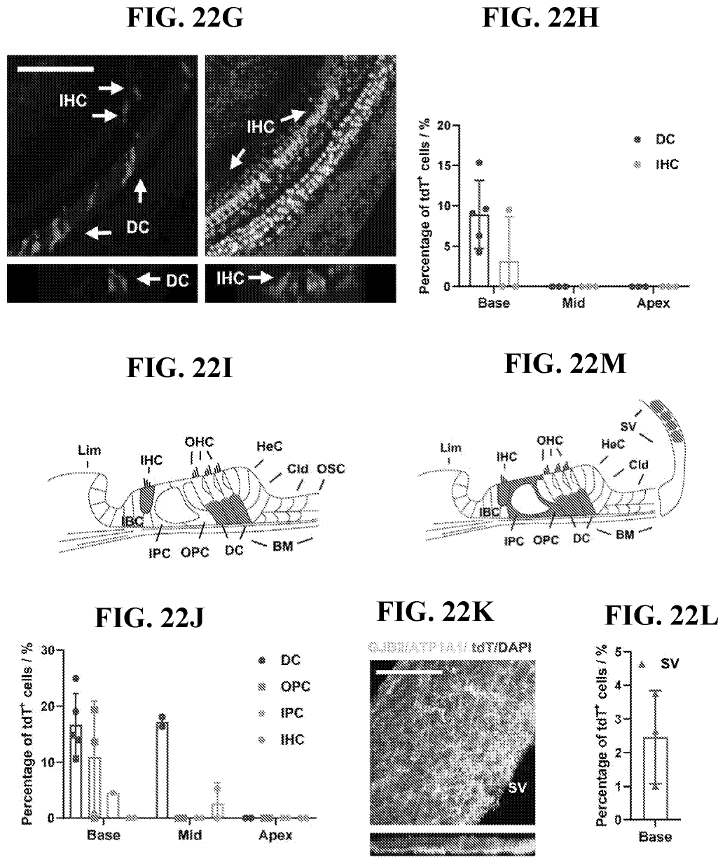

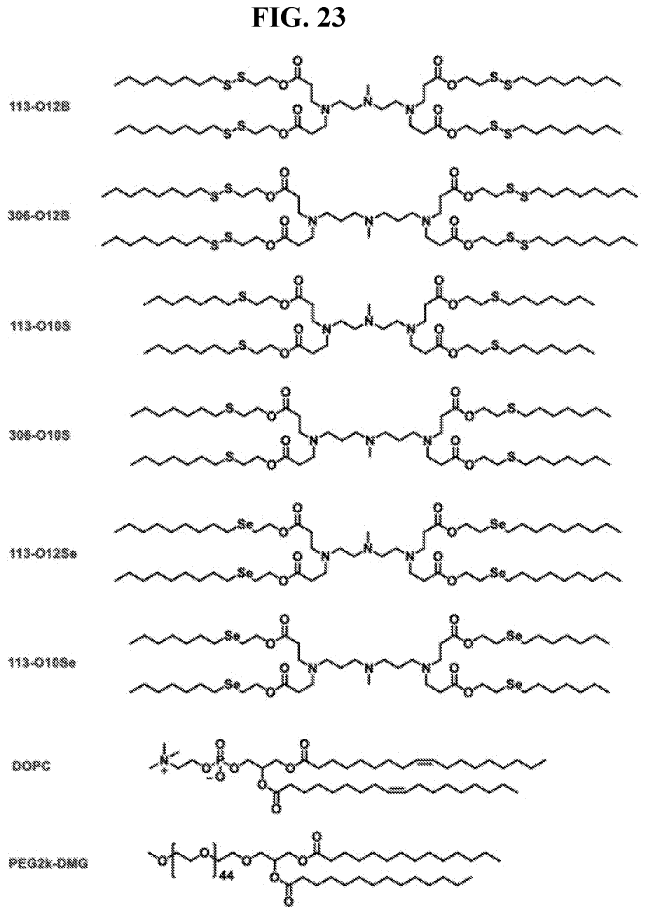

Using a combination of combinatorial chemistry and in vivo screening approach, recent studies showed that a group of multiple amine-containing lipids with short hydrophobic tails with the disulfide bond, such as 113-O12B and 306-O12B, were highly efficient for the systemic delivery of Cas9 mRNA-sgRNA in adult mice. A group of lipids with short tails containing either the disulfide bond (R-O12B) or chalcogens (R-O10X and R-O12X; FIG. 22A) were synthesized. Cas9 mRNA-sgRNA-loaded LNPs were fabricated using the synthesized lipid, cholesterol, DOPC, and PEG2k-DMG (multiple amine lipid/cholesterol/DOPC/PEG2k-DMG=16.7/4/2.1/1, weight ratio; multiple amine lipid/mRNA/sgRNA=15/1/1, weight ratio; FIG. 23). Typical TEM images of 113-O12B, 306-O12B, and 306-O10S LNPs showed that spherical particles with uniform were obtained (FIG. 22b and FIG. 21A) Further DLS analysis revealed that the hydrodynamic diameters of 113-O12B, 306-O12B, and 306-O10S LNPs were all in the range of 100-130 nm (FIG. 21B).

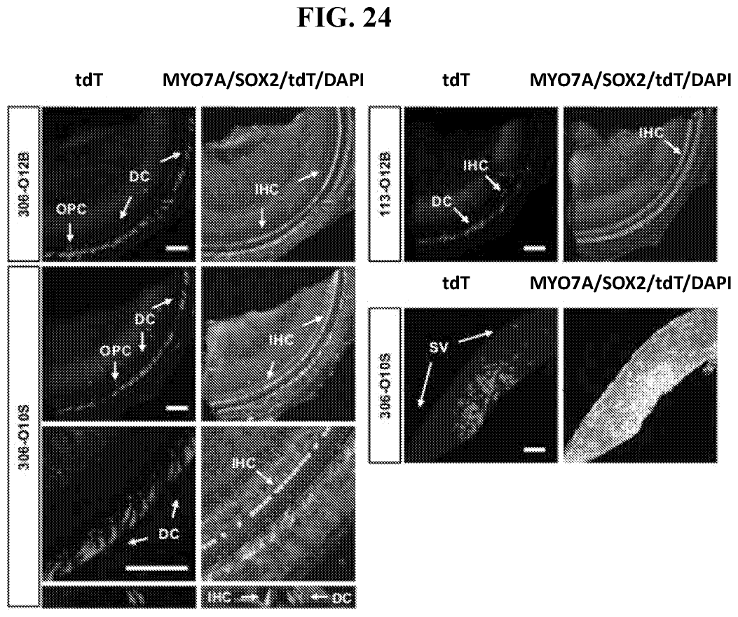

The Cas9 mRNA-sgRNA-loaded LNPs were then administered into adult Ai14 mouse cochlea through cochleostomy (FIG. 22C). The cochlea was collected 5-7 days post-injection, stained, and imaged. Contrary to the R-O17X LNPs, all the tested R-O12B, R-O10X, and R-O12X LNPs resulted in successful CRISPR-Cas9-mediated tdT expression. Specifically, strong tdT signals were observed in the base region of cochlea treated with Cas9 mRNA-sgRNA/306-O12B LNPs (FIG. 22D, FIG. 24). The tdT-positive cells were identified as DCs and OPCs. A small number of tdT-positive IPCs were also found. The tdT-positive cell populations were then quantified in the base, mid, and apex regions (FIG. 22E). 10.5% to 31.9% of tdT-positive DCs were found in the base region of five analyzed cochlea, with an average value of 22.3%. Three cochleae showed 2.8%-14.0% of tdT-positive OPCs in the base region. One cochlea had 6.25% tdT-positive IPCs in the base region. In the mid region, only one cochlea had 14.3% tdT-positive DCs. No OPCs or IPCs were transfected. In the apex region, no tdT signals were found. Overall, the Cas9 mRNA-sgRNA/306-O12B LNPs induced successful gene editing in the DCs, IPCs, and OPCs (FIG. 22F). It is noteworthy that, the variation between different injected cochleae was likely resulted from the cochleostomy surgical procedures. Due to the small size of the mouse inner ear, the technical perfection of cochleostomy in mouse models has been a long-standing challenge. However, it is anticipated that this technical issue can be addressed in pig models and non-human primates, which have similar cochlea size as humans and are much bigger than the mouse cochlea. Moreover, a gradual decrease of 306-O12B LNPs activity was observed from the base to mid and apex regions, indicating limitations of the passive diffusion of LNPs in the endolymph, which was consistent with the results of GFP mRNA- and Cre mRNA-loaded LNPs (FIG. 13, FIG. 17).

Cas9 mRNA-sgRNA/113-O12B LNPs induced tdT-positive signal expression in the base region of Ai14 cochlea (FIG. 22G, FIG. 24). Further analysis revealed that 4.3% to 15.4% DCs in the base region was found to be tdT-positive in five treated cochleae, with an average transfection efficacy of 8.9% (FIG. 22H). One cochlea had 10.0% of tdT-positive IHCs in the base; however, other cochleae did not show transfected IHCs. Compared with 306-O12B LNPs, no IPCs or OPCs were transfected by 113-O12B LNPs (FIG. 22I). Furthermore, no tdT signals were found in the mid and apex regions in any of the examined cochleae. tdT-positive cells were identified in the cochleae received with Cas9 mRNA-sgRNA/306-O10S LNPs (FIG. 24). Cells expressing tdT signals were mainly in the base region, and a small number of positive cells was also observed in the mid region. Specifically, in the base region, five analyzed cochleae showed 10.5%-25.0% tdT-positive DCs cells, with an average of 16.7% (FIG. 22J). 13.6% and 19.4% of tdT-positive OPCs were found in two of the analyzed cochleae, and one cochlea showed 4.5% tdT-positive IPCs. In the mid region, tdT-positive DCs were identified in two cochleae (16.4% and 18.1%), and 5.3% tdT-positive IHCs were found in one cochlea. In the apex region, no tdT signals were observed in any of the five cochleae. The cell types that were transfected by 306-O10S in the OC (FIG. 22M) were also observed in the 306-O12B and 113-O12B administered cochlea (FIG. 22F, FIG. 22I). All three LNP formulations lacked activity in the apex region, which could be resulted from inefficient diffusion in the endolymph.

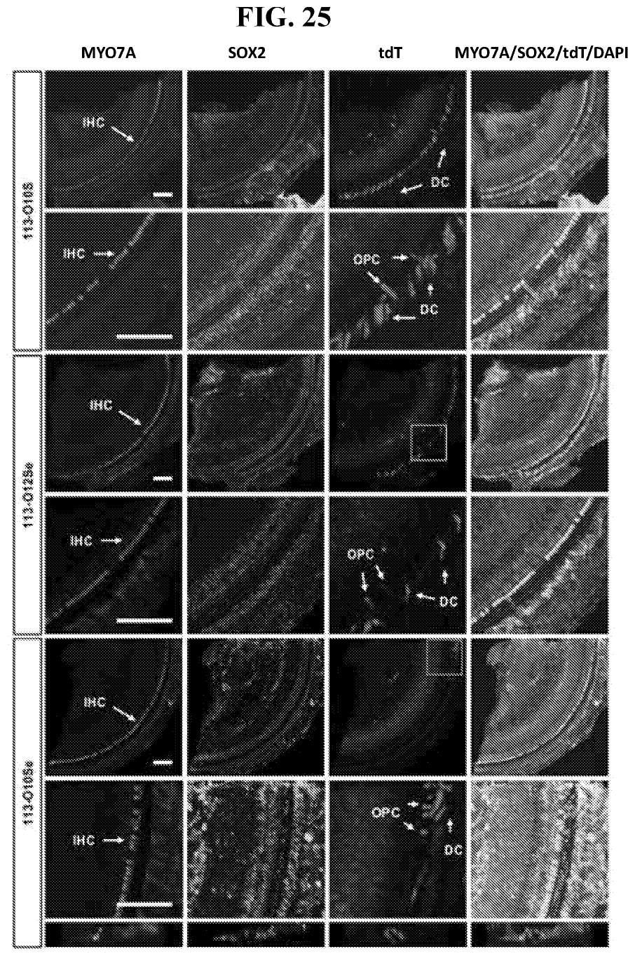

Surprisingly, tdT signals were also observed in the stria vascularis (SV) in the base region of the cochlea administered with Cas9 mRNA-sgRNA/306-O10S LNPs (FIG. 22K). 1.0% to 3.8% SV cells were found to be tdT positive in three cochleae, with an average number of 2.5% (FIG. 22L). These cells were identified as primarily intermediate cells and marginal cells in the SV. The SV is responsible for maintaining the ion composition of the endolymph and producing endocochlea potential in the SM. Targeting the functional cells in the SV can potentially create possibilities for treating inner ear disorders that involve dysregulated cochlea fluid homeostasis. The other three tested Cas9 mRNA-sgRNA/LNP formulations (i.e., 113-O10S, 113-O12Se, and 113-O10Se) also resulted in tdT expression in adult Ai14 mouse cochlea (FIG. 25). 113-O10S and 113-O12Se induced a larger number of tdT signals than the 113-O10Se. All tdT-positive cells were found in the base region, and no cells were transfected in the mid and apex regions. Moreover, the tdT expression cells were identified to be OPCs and DCs for all three LNP formulations.

It was demonstrated that the LNPs assembly from lipids with multiple amine heads (i.e., 113 and 306) and short tails (i.e., 012B, O10X, and 012X; X═S or Se) were capable of delivering Cas9 mRNA and sgRNA into the cochlea of adult Ai14 mice through cochleostomy. CRISPR-Cas9-mediated genome editing events were mainly observed in the base region, and a group of sensory and non-sensory cells in the OC were transfected, including the DCs, OPCs, IPC, and IHCs. One of the tested LNPs (i.e., 306-O10S), also induced tdT expression by the intermediate cells and marginal cells in the SV. Due to the flexibility of the CRISPR-Cas9 genome editing system, sgRNA targeting the functional genes in wild-type or diseased mouse models can be readily incorporated into these LNPs platforms with Cas9 mRNA for potential applications in mechanism study and therapeutics development for inner ear-related conditions.

In summary, it has been shown that the LNPs are a versatile and potent delivery platform that can deliver genome engineering protein and mRNA in the adult mouse cochlea. First of all, the disulfide bond-containing R-O16B and -R-N16B LNPs delivered (−30)GFP-Cre protein and enabled gene recombination in the BM and Lim of adult Ai14 mouse cochlea through canalostomy (FIGS. 5A to 5F). LNPs with the ester linkage (R-O16B) were more efficient than the LNPs with amide linkages (R-N16B). Second, the chalcogen-containing R-O17X LNPs were highly active in the delivery of Cre mRNA (FIGS. 9A to 9H). In the adult Ai14 mice, after canalostomy, 76-O17Se and 78-O17O LNPs induced gene recombination in BM and Lim. Strong tdT signals were observed throughout the cochlea, ranging from base to mid and apex regions. The incorporation of a series of excipients, including cationic 80-EC16, neutral HP-β-CD, and anionic SA, did not alter the cell specificity of the 76-O-17Se LNP formulations. Third, through cochleostomy, R-O17X LNPs delivered GFP mRNA in adult CD-1 mice cochlea and induced protein production in the OC sensory region (FIGS. 12A to 12E). The incorporation of cell-penetrating peptide TAT altered the cell specificity of R-O17X LNP formulations. The TAT-modified and unmodified 78-O17O LNPs were more efficient than the 76-O17Se LNP formulations. Fourth, Cre mRNA-loaded R-O17X LNPs led to successful gene recombination in the cochlea of adult Ai14 mice through cochleostomy (FIGS. 16A to 16E). Compared to the GFP mRNA/R-O17X LNPs in CD-1 mice, the Cre mRNA/R-O17X LNPs in Ai14 transfected different types of cell populations. TAT modification altered both 76-O17Se and 78-O17O LNPs cell specificity profiles. 76-O17Se-derived formulations were more potent than 78-O17O LNP formulations in regard to delivery efficacy. From the cochlea base to mid and apex, a gradual decrease in transfection efficacy was observed. Finally, as the R-O17X LNPs failed to deliver Cas9 mRNA-sgRNA to the cochlea after cochleostomy, a small group of disulfide bond- and chalcogen-containing R-O12B, R-O10X, and R-O12Se LNPs for CRISPR-Cas9 mRNA were fabricated for delivery (FIGS. 22A to 22J). Different LNP formulations showed different cell specificity and transfection efficacy, and the DCs and PCs in the OC are the most common tdT-positive cell types. 306-O10S also transfected cells in the SV. Quantification data showed that the majority of tdT-positive cells were located in the cochlea base region, which is like to due to inefficient diffusion of Cas9 mRNA-sgRNA/LNP formulations in the endolymph of SM duct. Variations were also observed between injected cochleae, which were believed to be associated with the technical procedures of cochleostomy. It is speculated that this would not be an issue in the later stage of LNP therapeutics formulation development when the cochlea of large animal models and non-human primates are targeted.

TABLE 3
Summary of mouse inner ear cell types that can be transfected by LNP-
based protein and mRNA delivery platforms identified in this study.

| LNP | Cargo | Administration | Mouse | Activity |
|---|---|---|---|---|
| 306-O16B | (−30)GFP-Cre | Canalostomy | Ai14 | Lim, BM |
| 76-O17Se | Cre mRNA | Canalostomy | Ai14 | Lim, BM |
| 78-O17O | | | | |
| 78-O17O | GFP mRNA | Cochleostomy | CD-1 | IPC, OPC |
| TAT-78-O17O | GFP mRNA | Cochleostomy | CD-1 | IHC, OHC |
| 76-O17Se | Cre mRNA | Cochleostomy | Ai14 | IPC, OPC, DC, IHC, |
| | | | | OHC, HeC, OSC |
| TAT-76-O17Se | Cre mRNA | Cochleostomy | Ai14 | Lim, IHC, DC, HeC |
| 78-O17O | Cre mRNA | Cochleostomy | Ai14 | Lim, IHC |
| TAT-78-O17O | Cre mRNA | Cochleostomy | Ai14 | OHC, OSC |
| 306-O12B | Cas9 mRNA-sgRNA | Cochleostomy | Ai14 | DC, OPC, IPC |
| 113-O12B | Cas9 mRNA-sgRNA | Cochleostomy | Ai14 | DC, IHC |
| 306-S10 (or | Cas9 mRNA-sgRNA | Cochleostomy | Ai14 | DC, OPC, IPC, IHC, |
| 306-O10S) | | | | SV |
| 113-S10 (or | Cas9 mRNA-sgRNA | Cochleostomy | Ai14 | DC, OPC |
| 113-O10S) | | | | |
| 113-Se12 (or | | | | |
| 113-O12Se) | | | | |
| 113-Se10 (or | | | | |
| 113-O10Se) | | | | |

Taken together, this study indicated that, first of all, the LNP delivery system can be engineered to enable both protein and mRNA-mediated genome engineering in the non-sensory and sensory regions of the adult mouse inner ear. The representative LNP formulations and their cochlea cell activities were summarized in Table 3. Second of all, the LNP delivery performance evaluated by the in vitro 2D cell culture model (e.g., HeLa-DsRed and mouse dermal fibroblasts used in this study) may not correlate well with the in vivo results, suggesting that a better in vitro model system that closely reflects the physiological property of the inner ear should be adapted in future studies when developing LNP-based protein or mRNA therapeutics. Third of all, the same type of mRNA/LNP formulation (e.g., Cre mRNA/76-O17Se) can behave very differently (e.g., cell specificity and transfection efficacy) in the adult mouse cochlea when different administration methods (i.e. canalostomy and cochleostomy) are employed. In the experiments, cells in BM and Lim were feasibly accessed by LNP formulations through canalostomy, and multiple types of cells located in the sensory region of OC could be transfected through cochleostomy. This is understandable as LNPs administered through canalostomy can travel through the perilymph and directly interact with the BM, and LNPs injected into the endolymph through cochleostomy are directly exposed to the OC, SV, and surrounding cells. This indicated that cochleostomy could be considered when cells located in the OC are primary targets; however, it should be also noted that cochleostomy is more invasive than canalostomy, and the cochleostomy surgical procedure can oftentimes lead to irreversible hair cell damage in mouse models. Third of all, LNP formulations carrying different cargos (e.g., Cre mRNA, GFP mRNA, or Cas9 mRNA-sgRNA) had different efficacy and cell specificity. This point should be carefully considered when fluorescent or bioluminescent reporter systems are utilized. If the biological properties of the LNPs loaded with reporters can retain when the cargo is replaced with bioactive functional components should be carefully examined. Moreover, how the genetic background of mouse models (e.g., Ai14 and CD-1) affects the property of the LNP-based delivery systems merits further investigation. Fourth of all, instead of simply altering the cellular internalization process, the modification of LNPs with TAT produced a profound effect on the cell selectivity of LNP formulations. It is hypothesized that specific ligand-receptor interactions were involved in directing LNPs to certain types of cochlea cells, which requires further investigation. But LNP modification with bioactive ligands could be a feasible and effective approach for altering and/or improving the performance of LNP delivery systems.

Overall, it has been demonstrated that a wide range of cell types in adult mouse inner ear can be transfected by LNP-mediated protein and mRNA delivery systems. Compared with previous study in neonatal mice, this study represents a step forward as it opens up new possibilities for genome engineering in the fully developed cochlea, which are more relevant to the cochlea of human newborns when most hearing disorders are diagnosed. The LNP formulations identified in this study that can mediate Cre and CRISPR-Cas9 mRNA delivery could be potentially used for mechanism study in the Cre-loxP mouse models and the development of CRISPR-based therapeutics for inner ear disorders. Further directions include illustrating the LNP structure-activity relationship in the cochlea, improving and optimizing LNPs formulations to achieve better efficacy and specificity, and incorporating sgRNA sequences targeting the disease-associated genes for the development of new genome editing therapeutics for hearing disorders.

### Example 8: Delivery of Pharmaceutical Agents to Humans

Pharmaceutical agents such as therapeutic proteins and therapeutic nucleic acids can be formulated with the lipids and LNP compositions provided herein for delivery into humans, such as to treat a congenital human disorder of the eye or ear. For example, to treat a genetic human disorder of the ear that is caused by the STRC gene, pharmaceutical agents that can modulate the STRC gene can be encapsulated by the LNP compositions provided herein. Then the composition can be injected via microinjection into a human neonate by canalostomy and cochleostomy.

### Example 9: mRNA Delivery in Mouse Retinas Using Novel Lipid Nanoparticles

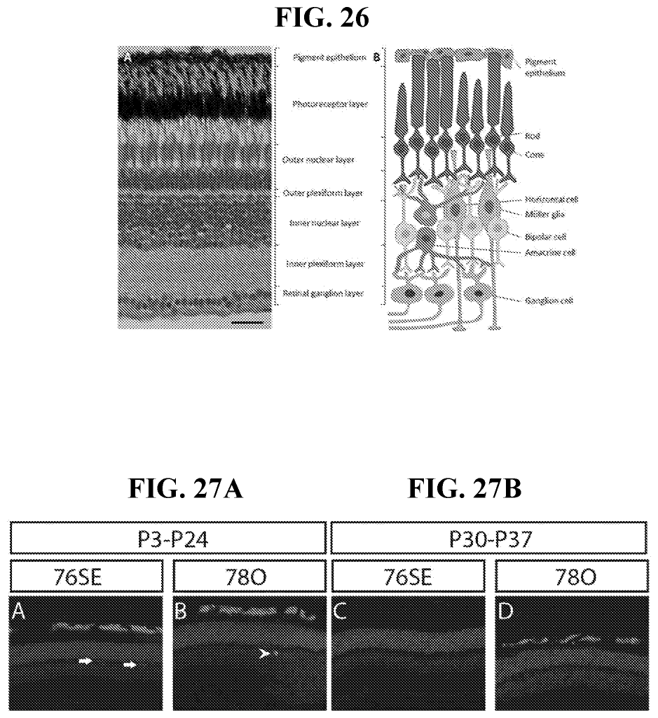

Neonatal and adult Ai9 or Ai11 mice homozygous for Rosa26-CAG-LSL-tdTomato transgene were used for the experiments. Five novel lipid nanoparticles (LNPs), 76SE (or 76-O17Se), 78O (or 78-O17O), 113-O12B, 306-O12B and 306-S10 (or 306-O10S) were tested to delivery either Cre mRNA or Cas9 mRNA/sgRNAs. FIG. 26 shows layers of cells in the eye. The LNP:RNA mixture was injected into subretinal space −0.5 μL/eye for neonates and 1 μL/eye for adults. Neonatal animals were sacrificed 3 weeks post-injection while the adult mice were sacrificed 1 week post injection. The eyes were fixed in 4% PFA, and the cornea and lenses were dissected out. The remaining eye cups were then cryoprotected with 30% sucrose and embedded in OCT. The eye cups were cryo-sectioned and stained with DAPI.

For delivery of Cre mRNA, 100 ng/μL Cre mRNA stock was freshly mixed with 1 μg/μL LNP stock and 1×PBS in 1:1:8 ratio. Our preliminary data showed that neonatal subretinal delivery of Cre mRNAs using either 76Se or 78O LNPs was able to induce tdTomato expression in retinal pigment epithelial (RPE) cells suggesting successful delivery of Cre-mRNAs into RPE cells by LNPs at P3 and P30 respectively, as shown in FIG. 27A. Some tdTomato expression was also noted in the outer plexiform layer (white arrows in FIG. 27A 76SE left) and the inner nuclear layer (white arrowhead n FIG. 27A 78O right). TdTomato expression in the RPE was only observed in one out of four adult eyes injected with 78O at dose of 1:1:8.

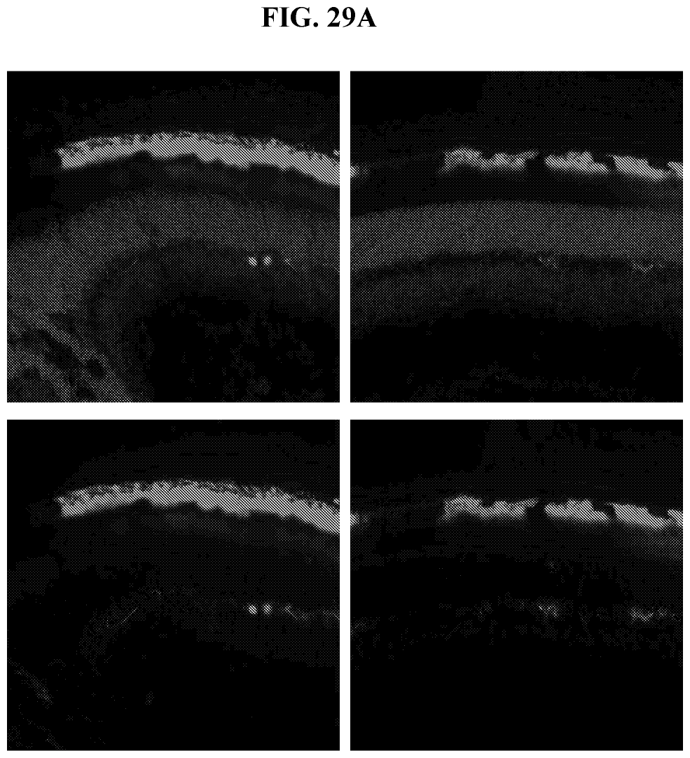

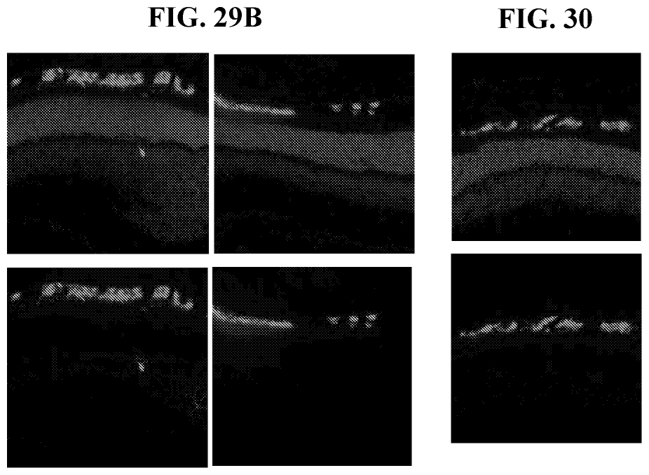

tdTomato expression was mainly induced in the RPE and not in the photoreceptors and tdTomato expression was not induced in adult mice injected with 76SE LNPs, a higher dose of mRNAs and LNPs (4:4:2) was attempted. The preliminary data showed that at higher dose, 2 out of 4 adult mice injected with Cre-mRNAs and 76SE LNPs showed tdTomato expression in a few of RPE cells. However, significant inflammation (marked by microphages) was noted, suggesting mechanical damage from the injection or higher toxicity of the LNPs. FIGS. 29A and 29B show expression of tdTomato in neonatal mouse RPE after Cre mRNA delivery by subretinal injection of 76Se and 78O LNP compositions respectively. The top panels how merge channels with DAPI, and the bottom panels show tdTomato staining. FIG. 30 shows 78O LNPs delivered Cre mRNA to RPE of adult mice

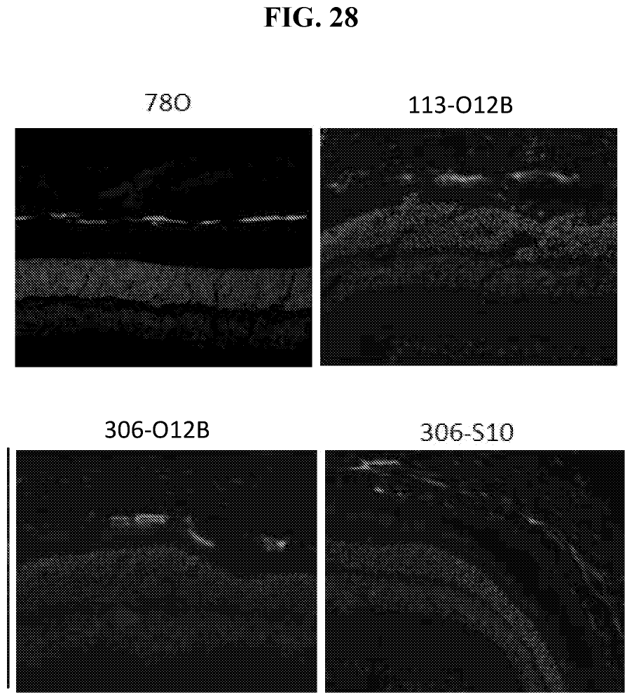

For delivery of Cas9 mRNA/sgRNA, Cas9 mRNA and sgRNA stock were freshly mixed with LNP in 1:1:15 ratio. 76SE or 78O LNP:RNA mixture was injected into the retinas of P0 pups. 113-012B, 306-012B or 306-S10 LNP:RNA mixture was injected in adult mice. Our preliminary data showed that LNP 78O, 113-012B, 306-012B and 306-S10 was able to deliver Cas9 mRNA/sgRNA into RPE cells, indicated by the expression of tdTomato in RPE cells in FIG. 28. No expression of tdTomato was observed in retinas injected with 76SE LNP:RNA.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A composition, comprising a pharmaceutical agent and a lipid composition comprising an ionizable lipid, wherein the ionizable lipid has an amine head group and at least one hydrophobic tail having a structure of Formula (A): 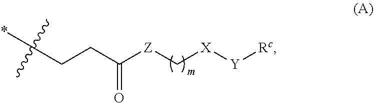
wherein:
*indicates the point of attachment to N;
X and Y are independently —CH₂—, —O—, —S—, or —Se—;
Z is N or O;
each m is independently 1, 2, 3, 4, or 5;
each R^{c} is independently an alkyl, or an alkenyl; and
the lipid composition is capable of delivering the pharmaceutical agent to a plurality of cell types in an ear.

2-4. (canceled)

5. The composition of claim 1, wherein the ionizable lipid comprises a structure of Formula (I): 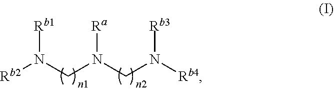
or a pharmaceutically acceptable salt thereof, wherein:
i) R^{a} is an alkyl;
ii) n1 and n2 are each independently 1, 2, 3, or 4; and
iii) R^{b1}, R^{b2}, R^{b3} and RM are each independently H, or 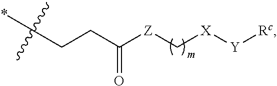
wherein at least one of R^{b1}, R^{b2}, R^{b3} and R^{b4} is not H.

6. The composition of claim 1, wherein the amine head group is selected from the group consisting of

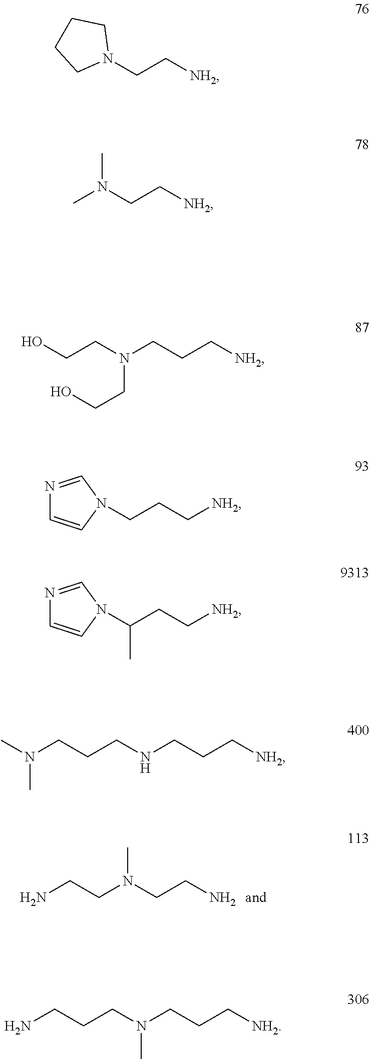

7. The composition of claim 1, wherein R^{c} is C₄-C₂₀ alkyl.

8. The composition of claim 1, wherein R^{c} is C₄-C₂₀ alkenyl.

9. The composition of claim 1, wherein the lipid composition further comprises a steroid.

10. The composition of claim 9, wherein the steroid is cholesterol.

11. The composition of claim 1, wherein the lipid composition further comprises a helper lipid.

12. The composition of claim 11, wherein the helper lipid is 1,2-dioleoyl-sn-glycero-3-phosphoethanolamine (DOPE) or 1,2-dioleoyl-sn-glycero-3-phosphocholine (DOPC).

13. (canceled)

14. The composition of claim 1, wherein the lipid composition further comprises a PEG conjugated lipid.

15. The composition of claim 14, wherein the PEG conjugated lipid is 1,2-distearoyl-sn-glycero-3-phosphoethanolamine-N-[methoxy(polyethylene glycol)-2000 (DSPE-PEG2k) or 1,2-dimyristoyl-rac-glycero-3-methoxypolyethylene glycol-2000 (DMG-PEG2k).

16. The composition of claim 1, wherein the lipid composition comprises a transactivator of transcription (TAT) peptide modification.

17. The composition of claim 1, wherein the lipid composition further comprises a steroid, a helper lipid, and a polymer conjugated polymer.

18. The composition of claim 17, wherein the ionizable lipid is present in the lipid composition at a weight percentage from about 30% to about 90%.

19-31. (canceled)

32. The composition of claim 1, wherein the pharmaceutical agent is a therapeutic agent, a gene modulating agent, or a vaccine.

33. The composition of claim 1, wherein the pharmaceutical agent comprises a polynucleotide, an oligonucleotide, a polypeptide, an oligopeptide, a small molecule compound, or any combination thereof.

34. (canceled)

35. (canceled)

36. The composition of claim 1, wherein the pharmaceutical agent comprises a heterologous endonuclease or a polynucleotide comprising a sequence that encodes the heterologous endonuclease.

37. The composition of claim 1, wherein the plurality of cell types is selected from the group consisting of inner hair cells (IHC), outer hair cells (OHC), Hensen cells (HeCs), Deiter cells (DC), outer sulcus cells (OSCs), inner pillar cells (IPC), and outer pillar cells (OPC).

38. The composition of claim 1, wherein the ionizable lipid comprises at least two hydrophobic tails, wherein not all hydrophobic tails are identical.

39. The composition of claim 1, wherein the ionizable lipid comprises at least two hydrophobic tails, wherein two or more hydrophobic tails are identical.

40. A method for delivering a pharmaceutical agent to an ear or an ear cell, the method comprising administering to a subject in need thereof an effective amount of the composition according to claim 1.

41.-47. (canceled)

48. A composition, comprising a pharmaceutical agent and a lipid composition comprising an ionizable lipid;
wherein the ionizable lipid has an amine head group and at least one hydrophobic tail having a structure of Formula (A): 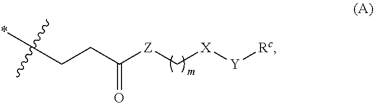
wherein:
*indicates the point of attachment to N;
X and Y are independently —CH₂—, —O—, —S—, or —Se—;
Z is N or O;
each m is independently 1, 2, 3, 4, or 5;
each R^{c} is independently an alkyl, or an alkenyl and
the lipid composition is capable of delivering the pharmaceutical agent to a plurality of regions in an eye.

49-88. (canceled)

89. A method for preferentially delivering a pharmaceutical agent to a target organ in a subject comprising administering a composition comprising: a pharmaceutical agent assembled with a lipid composition to the subject, wherein the lipid composition comprises a lipidoid having structural Formula (I): 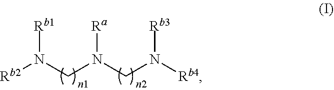
or a pharmaceutically acceptable salt thereof, wherein:
i) R^{a} is an alkyl (e.g., C₁-C₄ alkyl);
ii) n1 and n2 are each independently 1, 2, 3, or 4; and
iii) R^{b1}, R^{b2}, R^{b3} and R^{b4} are each independently H, or 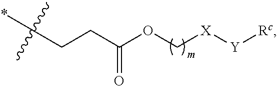
iv) wherein:
* indicates the point of attachment to N;
X and Y are independently —CH₂—, —O—, —S—, or —Se—;
each m is independently 1, 2, 3, 4, or 5; and
each R^{c} is independently an alkyl (e.g., C₄-C₂₀), or an alkenyl (e.g., C₄-C₂₀, 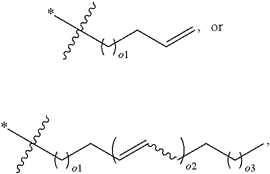
wherein each o1, o2, and o3 is independently an integer 1-10), * indicates the point of attachment to Y; and
v) wherein at least one of R^{b1}, R^{b2}, R^{b3} and R^{b4} is not H.

90.-156. (canceled)
